(19) United States
(12) Patent Application Publication
(10) Pub. No.: US 2019/0231787 A1
(43) Pub. Date: Aug. 1, 2019

(54) METHODS AND COMPOUNDS FOR TREATING ALCOHOL USE DISORDERS AND ASSOCIATED DISEASES

(71) Applicants: PHARMATROPHIX, INC., Menlo Park, CA (US); THE REGENTS OF THE UNIVERSITY OF CALIFORNIA, Oakland, CA (US); THE U.S. GOVERNMENT represented by THE DEPARTMENT OF VETERAN AFFAIRS, Washington, DE (US)

(72) Inventors: Stephen MASSA, Burlingame, CA (US); Frank M. LONGO, Menlo Park, CA (US); Dorit RON, San Francisco, CA (US)

(21) Appl. No.: 16/327,803

(22) PCT Filed: Aug. 25, 2017
(86) PCT No.: PCT/US17/48747

§ 371 Date: Feb. 22, 2019

Related U.S. Application Data
(60) Provisional application No. 62/379,686, filed on Aug. 25, 2016.
(60) Provisional application No. 62/396,081, filed on Sep. 16, 2016.

(51) Int. Cl. A61K 31/5375; A61P 25/32

(52) U.S. Cl. CPC A61K 31/5375; A61K 31/145; A61P 25/32

(57) ABSTRACT

Methods and compounds for treating alcohol use disorder and associated diseases. Included is the administering to a subject in need there of an effective amount of a compound having a modulating effect on p75NTR.

### CROSS-REFERENCE

This application claims priority to U.S. Provisional Application No. 62/379,686, filed on Aug. 25, 2016, and U.S. Provisional Application No. 62/396,081, filed on Sep. 16, 2016, the entire contents of each of which are incorporated herein by reference.

### STATEMENT AS TO FEDERALLY SPONSORED RESEARCH

This invention was made with government support under grant nos. R01 AA016848 and R37 AA016848 awarded by the National Institutes of Health. The government has certain rights in the invention.

### BACKGROUND OF THE INVENTION

Brain-derived neurotrophic factor (BDNF) is a member of the neurotrophins family of growth factors that plays an important role in the survival of neurons, and in the formation and maturation of synapses. BDNF also contributes to long-term potentiation (LTP), enhancement of neurotransmitter release, and alterations of channel function and spine morphology. Binding of BDNF to receptor tyrosine kinase tropomyosin receptor kinase B (TrkB) leads to activation of various intracellular signaling pathways. BDNF can also bind, with lower affinity, with the p75 neurotrophin receptor (p75NTR), a member of the tumor necrosis factor receptor superfamily.

It has been suggested that BDNF signaling keeps alcohol intake in moderation. BDNF and its receptor, TrkB, are part of an endogenous system that keeps alcohol drinking in moderation. Malfunctioning of the BDNF signaling pathway has been linked to alcohol use disorder and associated diseases.

Traditionally, treatment for alcohol use disorder and associated diseases includes prescribing medication and behavioral therapy. Medicines approved by the Food and Drug Administration (FDA) to treat alcohol dependence include disulfiram (Antabuse®), oral naltrexone, extended-release naltrexone (Vivitrol®), and acamprosate (Campral®). Despite advances in the field, the available therapies are not effective for all patients and the majority of people with alcohol use disorder and associated diseases are left untreated.

Accordingly, there is a current need for new agents for treating alcohol use disorder and associated diseases. The present disclosure addresses this deficiency with compounds and compositions for use in treating alcohol use disorder and associated diseases.

### SUMMARY OF THE INVENTION

The disclosure provides a method for treating an alcohol use disorder, comprising administering to a subject in need thereof an effective amount of a compound represented by Formula III:

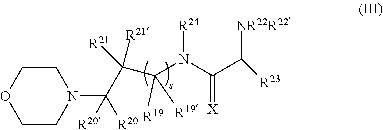

or a salt thereof, wherein: X is CH₂, NH, O or S; s is 0, 1, 2, 3 or 4; each of R¹⁹, R^{19′}, R²⁰, R^{20′}, R²¹, R^{21′}, R²², R^{22′} and R²⁴ is independently selected at each occurrence from hydrogen and optionally substituted alkyl; or R²⁰ and R^{20′} taken together form ═O, ═S, or ═CH₂; or R²⁰ and R²¹ taken together with the atoms to which they are attached form an optionally substituted cycloalkyl; or R²⁰ and R²¹ taken together with the atoms to which they are attached form an optionally substituted aryl; or R¹⁹ and R²⁰ taken together with the atoms to which they are attached form an optionally substituted cycloalkyl; or R¹⁹ and R²⁰ taken together with the atoms to which they are attached form an optionally substituted aryl; and R²³ is hydrogen, optionally substituted alkyl, optionally substituted cycloalkyl or optionally substituted aryl.

In certain embodiments, for a compound or salt of Formula III, s is 0, 1 or 2, such as s is 0. In certain embodiments, for a compound or salt of Formula III, X is NH, O or S, such as X is O.

In certain embodiments, for a compound or salt of Formula III, R²⁰ and R^{20′} are independently selected from hydrogen and optionally substituted C₁-C₆ alkyl, such as each of R²⁰ and R^{20′} are hydrogen. In certain embodiments, for a compound or salt of Formula III, R²¹ and R^{21′} are independently selected from hydrogen and optionally substituted C₁-C₆ alkyl, such as each of R²¹ and R^{21′} are hydrogen. In certain embodiments, for a compound or salt of Formula III, R²² and R^{22′} are independently selected from hydrogen and optionally substituted C₁-C₆ alkyl, such as each of R²² and R^{22′} are hydrogen.

In certain embodiments, for a compound or salt of Formula III, R²³ is selected from hydrogen, optionally substituted C₁-C₆ alkyl, optionally substituted cycloalkyl and optionally substituted aryl such as R²³ is selected from optionally substituted C₁-C₆ alkyl. In certain embodiments, R²³ is represented by the structure:

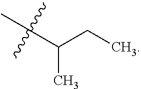 [SMILES: *C(C)CC]

In certain embodiments, for a compound or salt of Formula III, R²⁴ is hydrogen or optionally substituted C₁-C₆ alkyl, such as R²⁴ is hydrogen.

In certain embodiments, the compound of Formula III is represented by the structure:

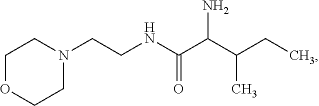 [SMILES: CCC(C)C(N)C(=O)NCCN1CCOCC1]

or a salt thereof. The compound of Formula III may be represented by the structure:

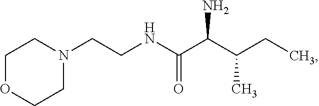 [SMILES: CC[C@H](C)[C@H](N)C(=O)NCCN1CCOCC1]

or a salt thereof. The salt of Formula III may be represented by Formula X:

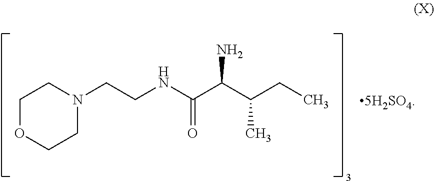

In certain embodiments, for the methods of treating an alcohol use disorder described herein, said subject has a predisposition to alcoholism. In certain embodiments, said alcohol use disorder comprises drinking greater than three alcoholic beverages a day. In certain embodiments, said alcohol use disorder comprises drinking alcoholic beverages three or more days in a week.

In certain embodiments, for the methods of treating an alcohol use disorder described herein, said subject exhibits one or more symptoms of an alcohol use disorder selected from: hepatic steatosis, alcoholic hepatitis, cirrhosis, gastritis, stomach ulcers, esophageal ulcers, interference with absorption of B vitamins and other nutrients, pancreatitis, high blood pressure, enlarged heart, heart failure, stroke, atrial fibrillation, cardiovascular disease, hypoglycemia, diabetes, erectile dysfunction, interruption of menstruation, nystagmus, weakness of eye muscles, paralysis of eye muscles, thiamine deficiency, dementia, miscarriage, fetal alcohol syndrome, osteoporosis, damaged bone marrow, low platelet count, numbness and pain in body, disordered thinking, short-term memory loss, weakened immune system, infectious disease, cancer, anemia, depression, seizures, gout, nerve damages, and combinations thereof.

In certain embodiments, for the methods of treating an alcohol use disorder described herein, said subject is a participant in an alcohol use management program. In certain embodiments, for the methods of treating an alcohol use disorder described herein, said alcohol use disorder involves increased synaptosomal localization of p75NTR. Said p75NTR may be localized in the DLS.

In certain embodiments, administering a compound or salt of Formula III or X to the subject modulates p75NTR levels of said subject. In certain embodiments, administering a compound or salt of Formula III or X to the subject attenuates alcohol intake of said subject as compared with the frequency of alcohol intake prior to administering said compound or salt. In certain embodiments, administering a compound or salt of Formula III or X to the subject attenuates alcohol intake of said subject as compared with the amount of alcohol intake before administering said compound or salt thereof. Administering said compound or salt may attenuate alcohol intake of said subject by about 10% or more, about 20% or more, about 30% or more, about 40% or more, or about 50% or more as compared with the amount of alcohol intake before administering said compound or salt thereof.

In certain embodiments, a compound or salt of Formula III or X is administered to the subject at least about once a week, such as at least about twice a week. In certain embodiments, a compound or salt of Formula III or X is administered to the subject daily or every other day. In certain embodiments, a compound or salt of Formula III or X is administered to the subject before, during, and/or after a trigger event, wherein the trigger event may be, for example, attending an event with alcoholic beverages, exposure to a stressful situation, and the end of a work day. In certain embodiments, administering said compound or salt of Formula III or X does not affect consumption of food or non-alcoholic beverages, e.g., does not attenuate or increase consumption of food or non-alcoholic beverages.

In certain embodiments, the methods described herein may be used to treat a disorder selected from alcohol abuse, alcohol dependence, and alcoholism. In certain embodiments, said disorder is alcohol abuse.

The methods described herein may further comprise administering one or more additional therapeutic agents selected from, for example, disulfiram (Antabuse®), oral naltrexone, extended-release naltrexone (Vivitrol®), and acamprosate (Campral®). The methods described herein may further comprise administering behavioral therapy to said subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts a study setup where rats were treated with specific alcohol concentrations and were infused with BDNF at various stages.

FIG. 1B is a schematic drawing of coronal sections of a rat brain showing the placement of bilateral infusion sites in the dorsolateral striatum (DLS).

FIG. 1C is a line graph that shows that intra-DLS administration of BDNF does not decrease the number of alcohol deliveries. Data are expressed as the mean±SEM of the number of alcohol deliveries. n=7 per treatment.

FIG. 1D is a line graph that shows that intra-DLS administration of BDNF does not decrease the amount of alcohol consumed. Data are expressed as the mean±SEM of the alcohol consumed in grams per kilogram. n=7 per treatment.

FIG. 2A is a schematic drawing of coronal sections of the rat brain showing the DLS in black.

FIG. 2B is a schematic representation of dissection timeline. Alc, Alcohol; W, water.

FIG. 2C provides a digital image of a Western blot and corresponding histogram of p75NTR and TrkB levels in total homogenates where the rats received IA20%-2BC (black) or water only (white) for 7 weeks, and the DLS slices of the high-drinking rats (alcohol intake equal to or >3.5 g/kg/24 h) were dissected immediately after the last 30 min (binge). The histogram depicts the mean ratio of TrkB or p75NTR and actin±SEM and, and values are expressed as percentages of water controls. n=10-11 per time point of dissection.

FIG. 2D provides a digital image of a Western blot and corresponding histogram of p75NTR and TrkB levels in the synaptosomal fractions where the rats received IA20%-2BC (black) or water only (white) for 7 weeks, and the DLS slices of high-drinking rats (alcohol intake equal to or >3.5 g/kg/24 h) were dissected immediately after the last 30 min (binge). The histogram depicts the mean ratio of TrkB or p75NTR and actin±SEM and, and values are expressed as percentages of water controls. ***p<0.0001 versus water (unpaired t test). n=10-11 per time point of dissection.

FIG. 2E provides a digital image of a Western blot and corresponding histogram of p75NTR and TrkB levels in total homogenates where the rats received IA20%-2BC (black) or water only (white) for 7 weeks, and the DLS slices of high-drinking rats (alcohol intake equal to or >3.5 g/kg/24 h) were dissected at the end of the last 24 h drinking session (end). The histogram depicts the mean ratio of TrkB or p75NTR and actin±SEM and, and values are expressed as percentages of water controls. n=8 per time point of dissection.

FIG. 2F provides a digital image of a Western blot and corresponding histogram of p75NTR and TrkB levels in the synaptosomal fractions where the rats received IA20%-2BC (black) or water only (white) for 7 weeks, and the DLS slices of high-drinking rats (alcohol intake equal to or >3.5 g/kg/24 h) were dissected at the end of the last 24 h drinking session (end). The histogram depicts the mean ratio of TrkB or p75NTR and actin±SEM and, and values are expressed as percentages of water controls. n=8 per time point of dissection.

FIG. 2G provides a digital image of a Western blot and corresponding histogram p75NTR and TrkB levels in total homogenates where the rats received IA20%-2BC (black) or water only (white) for 7 weeks. The DLS slices of high-drinking rats (alcohol intake equal to or >3.5 g/kg/24 h) were dissected after 24 h of withdrawal (WD). The histogram depicts the mean ratio of TrkB or p75NTR and actin±SEM and, and values are expressed as percentages of water controls. n=8 per time point of dissection.

FIG. 2H provides a digital image of a Western blot and corresponding histogram p75NTR and TrkB levels in the synaptosomal fractions where the rats received IA20%-2BC (black) or water only (white) for 7 weeks, and the DLS slices of high-drinking rats (alcohol intake equal to or >3.5 g/kg/24 h) were dissected after 24 h of withdrawal (WD). The histogram depicts the mean ratio of TrkB or p75NTR and actin±SEM and, and values are expressed as percentages of water controls. ***p<0.0001 versus water (unpaired t test). n=8 per time point of dissection.

FIG. 3A provides a digital image of a Western blot and corresponding histogram of p75NTR and TrkB levels in total homogenate where the rats received a systemic administration of alcohol (Alc; 1.5 g/kg, i.p.; black) or saline (Sal; white), and the DLS was dissected 30 min later. The histogram shows mean ratio of TrkB or p75NTR and actin±SEM, and values are expressed as percentages of water controls. n=8 per treatment.

FIG. 3B provides a digital image of a Western blot and corresponding histogram of p75NTR and TrkB levels in the synaptosomal fraction where the rats received a systemic administration of alcohol (Alc; 1.5 g/kg, i.p.; black) or saline (Sal; white), and the DLS was dissected 30 min later. The histogram shows mean ratio of TrkB or p75NTR and actin±SEM, and values are expressed as percentages of water controls. n=8 per treatment.

FIG. 3C provides a digital image of a Western blot and corresponding histogram of p75NTR and TrkB levels in total homogenate where the rats received continuous access to 10% alcohol (CA10%; black) or water only (white) for 21 d, and the DLS was dissected immediately after the last drinking session. The histogram shows mean ratio of TrkB or p75NTR and actin±SEM, and values are expressed as percentages of water controls. n=8 per drinking regimen.

FIG. 3D provides a digital image of a Western blot and corresponding histogram of p75NTR and TrkB levels in the synaptosomal fraction where the rats received continuous access to 10% alcohol (CA10%; black) or water only (white) for 21 d, and the DLS was dissected immediately after the last drinking session. The histogram shows mean ratio of TrkB or p75NTR and actin±SEM, and values are expressed as percentages of water controls. n=8 per drinking regimen.

FIG. 3E provides a digital image of a Western blot and corresponding histogram of p75NTR and TrkB levels in total homogenate where the rats received 7 weeks of IA of 1% sucrose (Suc) or water only, and the DLS was dissected immediately after the last 30 min drinking session. The histogram shows mean ratio of TrkB or p75NTR and actin±SEM, and values are expressed as percentages of water controls. n=4 per drinking regimen.

FIG. 3F provides a digital image of a Western blot and corresponding histogram of p75NTR and TrkB levels in the synaptosomal fraction where the rats received 7 weeks of IA of 1% sucrose (Suc) or water only, and the DLS was dissected immediately after the last 30 min drinking session. The histogram shows mean ratio of TrkB or p75NTR and actin±SEM, and values are expressed as percentages of water controls. n=4 per drinking regimen.

FIG. 4A is a schematic drawing of coronal sections of the rat brain showing the DMS in black.

FIG. 4B provides a digital image of a Western blot and corresponding histogram of p75NTR and TrkB levels in total homogenates where the rats received IA20%-2BC for 7 weeks (black) or water only (white), and the DMS was dissected immediately after the last 30 min drinking session (binge). The histograms show the mean ratio of TrkB or p75NTR and actin±SEM, and values are expressed as percentages of water controls. n=8 per time point of dissection.

FIG. 4C provides a digital image of a Western blot and corresponding histogram of p75NTR and TrkB levels in the synaptosomal fraction where the rats received IA20%-2BC for 7 weeks (black) or water only (white), and the DMS was dissected immediately after the last 30 min drinking session (binge). The histograms show the mean ratio of TrkB or p75NTR and actin±SEM, and values are expressed as percentages of water controls. n=8 per time point of dissection.

FIG. 4D provides a digital image of a Western blot and corresponding histogram of p75NTR and TrkB levels in total homogenates where the rats received IA20%-2BC for 7 weeks (black) or water only (white), and the DMS was dissected or at the end of the 24 h drinking session (end). The histograms show the mean ratio of TrkB or p75NTR and actin±SEM, and values are expressed as percentages of water controls. n=8 per time point of dissection.

FIG. 4E provides a digital image of a Western blot and corresponding histogram of p75NTR and TrkB levels in the synaptosomal fraction where the rats received IA20%-2BC for 7 weeks (black) or water only (white), and the DMS was dissected or at the end of the 24 h drinking session (end). The histograms show the mean ratio of TrkB or p75NTR and actin±SEM, and values are expressed as percentages of water controls. n=8 per time point of dissection.

FIG. 5A shows the DLS that was bilaterally infused with Ltvshp75NTR or Ltv-shSCR. The DLS was costained with anti-GFP (green) and anti-NeuN (red) or costained with anti-GFP and anti-GFAP antibodies. The left image depicts the specificity of the site of infection. The right images depict Ltv-shp75NTR infection of neurons (costaining of GFP with NeuN; top) but not glia (costaining GFP with GFAP; bottom).

FIG. 5B provides a digital image of a Western blot and corresponding histogram of p75NTR and GAPDH protein levels where Ltvshp75NTR or Ltv-shSCR was bilaterally infused at a titer of 2×10⁷ pg/ml into the DLS. Data are expressed as mean ratio of p75NTR and GAPDH±SEM and presented as percentages of Ltv-shSCR control. **p<0.001 versus Ltv-shSCR (unpaired t test). n=4 per treatment.

FIG. 5C is a schematic representation of the behavioral experiment where the rats underwent IA20%-2BC alcohol for 7 weeks, the high-drinking rats (baseline level of alcohol intake equal or higher than 3.5 g/kg/24 h) received a bilateral infusion of Ltv-shSCR or Ltv-shp75NTR into the DLS, and after 1 week of recovery, the alcohol-drinking procedure resumed.

FIG. 5D is a histogram that demonstrates alcohol intake (grams per kilogram per 30 min) for 4 weeks after viral infection. Results are expressed as mean±SEM. *p<0.01. n=7 per treatment.

FIG. 5E is a histogram that demonstrates water intake (milliliters per kilogram per 30 min) for 4 weeks after viral infection. Results are expressed as mean±SEM. n=7 per treatment.

FIG. 6A is a series of schematic drawings of coronal sections of the rat brain showing the placement of bilateral infusion sites in the DLS.

FIG. 6B is a histogram showing alcohol (grams per kilogram per 30 min) intake after LM11A-31 or vehicle infusion. *p<0.05 versus vehicle (paired t test). n=11 per treatment.

FIG. 6C is a histogram showing water (milliliters per kilogram per 30 min; C) intake after LM11A-31 or vehicle infusion. n=11 per treatment.

FIG. 6D is a histogram showing sucrose (milliliters per kilogram per 30 min) consumption where after 1 week of withdrawal from alcohol consumption, rats underwent 2 weeks of IA-2BC of 1% sucrose and then received intra-DLS administration of LM11A-31 (30 μg/μl) or vehicle. The results are expressed as mean±SEM. n=10 per treatment.

FIG. 6E is a histogram showing water (milliliters per kilogram per 30 min) consumption where after 1 week of withdrawal from alcohol consumption, rats underwent 2 weeks of IA-2BC of 1% sucrose and then received intra-DLS administration of LM11A-31 (30 μg/μl) or vehicle. The results are expressed as mean±SEM. n=10 per treatment.

FIG. 7A is a histogram showing alcohol (grams per kilogram per 30 min) intake where the rats underwent an IA-2BC paradigm with 20% alcohol for 7 weeks, and LM11A-31 (50-150 mg/kg) or vehicle (0.9% NaCl) was administered intraperitoneally 2 hours before the beginning of the drinking session. The results are expressed as mean±SEM. *p<0.01 versus vehicle (one-way ANOVA with SNK post hoc test). Vehicle, n=24 per dose; LM11A-31, n=12 per dose.

FIG. 7B is a histogram showing water (milliliters per kilogram per 30 min) intake where the rats underwent an IA-2BC paradigm with 20% alcohol for 7 weeks, and LM11A-31 (50-150 mg/kg) or vehicle (0.9% NaCl) was administered intraperitoneally 2 hours before the beginning of the drinking session. The results are expressed as mean±SEM. Vehicle, n=24 per dose; LM11A-31, n=12 per dose.

FIG. 7C is a histogram showing sucrose (milliliters per kilogram per 30 min) intake where the rats underwent an IA-2BC paradigm with 1% sucrose for 7 weeks, and LM11A-31 (50-150 mg/kg) or vehicle (0.9% NaCl) was administered intraperitoneally 2 hours before the beginning of the drinking session. The results are expressed as mean±SEM. n=7-8 per treatment.

FIG. 7D is a histogram showing water (milliliters per kilogram per 30 min) intake where the rats underwent an IA-2BC paradigm with 1% sucrose for 7 weeks, and LM11A-31 (50-150 mg/kg) or vehicle (0.9% NaCl) was administered intraperitoneally 2 hours before the beginning of the drinking session. The results are expressed as mean±SEM. n=7-8 per treatment.

### DETAILED DESCRIPTION OF THE INVENTION

### Overview

Malfunctioning of the BDNF signaling pathway has been linked to alcohol use disorder and associated diseases. In particular, in subjects with alcohol use disorder and associated diseases, it has been observed that the BDNF/TrkB pathway in the dorsolateral striatum (DLS) ceases to participate in mechanisms that gate alcohol self-administration. In conjunction with BDNF pathway malfunction, it is observed that synaptosomal p75NTR levels increase in subjects with alcohol use disorder and associated diseases and contribute to mechanisms that drive excessive alcohol use.

In certain aspects, the disclosure provides a modulator of p75NTR signaling within the DLS, and thereby treat or prevent alcohol use disorder and associated diseases

### Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application. Generally the term “about”, as used herein when referring to a measurable value such as an amount of weight, time, dose, etc. is meant to encompass in one example variations of ±20% or +10%, in another example±5%, in another example±1%, and in yet another example±0.1% from the specified amount, as such variations are appropriate to perform the disclosed method.

The term “alkyl,” alone or in combination, refers to an optionally substituted straight-chain or branched-chain alkyl radical having from 1 to 20 carbon atoms. The term also includes optionally substituted straight-chain or branched-chain alkyl radicals having from 1 to 6 carbon atoms as well as those having from 1 to 4 carbon atoms. Examples of alkyl radicals include methyl, ethyl, n-propyl, isopropyl, n-butyl, isobutyl, sec-butyl, tert-butyl, tert-amyl, pentyl, hexyl, heptyl, octyl and the like. “Branched” refers to an alkyl group in which a lower alkyl group, such as methyl, ethyl or propyl, is attached to a linear alkyl chain. “Lower alkyl” refers to an alkyl group having 1 to 8 carbon atoms (i.e., a C₁₋₈ alkyl), e.g., 1, 2, 3, 4, 5, 6, 7, or 8 carbon atoms. “Higher alkyl” refers to an alkyl group having about 10 to 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁-8 branched-chain alkyls. Alkyl groups can be optionally substituted.

The term “alkenyl,” alone or in combination, refers to an optionally substituted straight-chain or branched-chain hydrocarbon radical having one or more carbon-carbon double-bonds and having from 2 to 18 carbon atoms. Alkenyl includes optionally substituted straight-chain or branched-chain hydrocarbon radicals having one or more carbon-carbon double bonds and having from 2 to 6 carbon atoms such as from 2 to 4 carbon atoms. Examples of alkenyl radicals include ethenyl, propenyl, butenyl, 1,4-butadienyl and the like. Suitable alkenyl groups include allyl. The terms “allylic group” or “allyl” refer to the group —CH₂HC═CH₂ and derivatives thereof formed by substitution. Thus, the terms alkenyl and/or substituted alkenyl include allyl groups, such as but not limited to, allyl, methylallyl, di-methylallyl, and the like. The term “allylic position” or “allylic site” refers to the saturated carbon atom of an allylic group. Thus, a group, such as a hydroxyl group or other substituent group, attached at an allylic site can be referred to as “allylic.” “l-alkenyl” refers to alkenyl groups where the double bond is between the first and second carbon atom.

The term “alkynyl,” alone or in combination, refers to an optionally substituted straight-chain or branched-chain hydrocarbon radical having one or more carbon-carbon triple-bonds and having from 2 to 12 carbon atoms. Alkynyl includes optionally substituted straight-chain or branched-chain hydrocarbon radicals having one or more carbon-carbon triple bonds and having from 2 to 6 carbon atoms such as from 2 to 4 carbon atoms. Examples of alkynyl radicals include ethynyl, propynyl, butynyl and the like. “l-alkynyl” refers to alkynyl groups where the triple bond is between the first and second carbon atom.

“Cycloalkyl” refer to a non-aromatic mono- or multicyclic ring system of 3 to 10 carbon atoms, e.g., 3, 4, 5, 6, 7, 8, 9, or 10 carbon atoms, such as from 3 to 6 carbon atoms. The cycloalkyl group can be optionally partially unsaturated. The cycloalkyl group also can be optionally substituted as defined herein. Representative monocyclic cycloalkyl rings include, but are not limited to, cyclopropyl, cyclobutyl, cyclopentyl, cyclohexyl, cycloheptyl, and the like. Further, the cycloalkyl group can be optionally substituted with a linking group, such as an alkylene group as defined hereinabove, for example, methylene, ethylene, propylene, and the like. In such cases, the cycloalkyl group can be referred to as, for example, cyclopropylmethyl, cyclobutylmethyl, and the like. Additionally, multicyclic cycloalkyl rings include adamantyl, octahydronaphthyl, decalin, camphor, camphane, and noradamantyl.

The term “heterocycloalkyl” refers to a cyclic group of 3 to 6 atoms, or 3 to 10 atoms, containing at least one heteroatom. In one aspect, these groups contain 1 to 3 heteroatoms. Suitable heteroatoms include, for example, oxygen, sulfur, and nitrogen. Heterocyclic groups may be attached through any substitutable atom, such as a nitrogen or through a carbon atom in the ring. Suitable heterocyclic groups include pyrrolidinyl, morpholino, morpholinoethyl, and pyridyl. Such groups may be substituted.

The term “aryl” refers to aromatic groups which have 5-14 ring atoms and at least one ring having a conjugated pi electron system and includes carbocyclic aryl, heteroaryl and biaryl groups, all of which may be optionally substituted. The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. In particular embodiments, the term “aryl” includes cyclic aromatic comprising about 5 to about 10 carbon atoms, e.g., 5, 6, 7, 8, 9, or 10 carbon atoms, and including 5- and 6-membered hydrocarbon and heterocyclic aromatic rings. Examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like, all optionally substituted. Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto. Unless otherwise specified, substituents on an aryl group may be independently selected at each occurrence from alkyl, aryl, aralkyl, hydroxyl, alkoxyl, haloalkyl, aryloxyl, aralkyloxyl, carboxyl, acyl, halo, nitro, alkoxycarbonyl, aryloxycarbonyl, aralkoxycarbonyl, acyloxyl, acylamino, carbamoyl, alkylcarbamoyl, dialkylcarbamoyl, arylthio, alkylthio, and —NR′R″, wherein R′ and R″ can each be independently hydrogen, alkyl, substituted alkyl, aryl, substituted aryl, and aralkyl.

A structure represented generally by a formula such as:

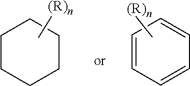

as used herein refers to a 6-carbon ring structure comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure:

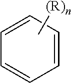

wherein n is an integer from 0 to 2 comprises compound groups including, but not limited to:

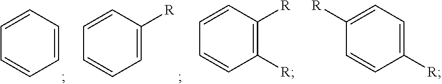

and the like.

The structure:

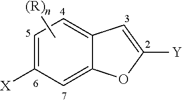

wherein n is one (1) comprises compound groups including:

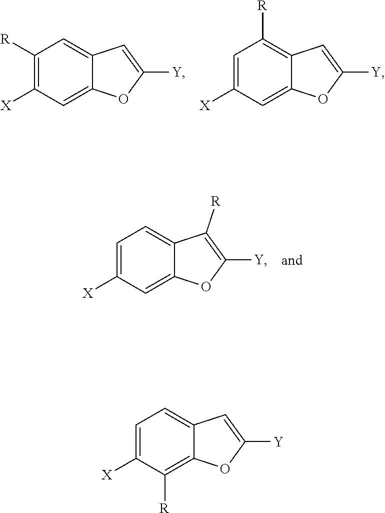

wherein the one (1) R substituent can be attached at any carbon on the benzofuran parent structure not occupied by another designated substituent, as in this case carbon 6 is substituted by X and carbon 2 is substituted by Y.

A dashed line representing a bond in a cyclic ring structure indicates that the bond can be either present or absent in the ring.

“Carbocyclic aryl” groups are groups wherein the ring atoms on the aromatic ring are carbon atoms. Carbocyclic aryl groups include monocyclic carbocyclic aryl groups and polycyclic or fused compounds such as optionally substituted naphthyl groups.

“Heteroaryl” groups are groups having from 1 to 4 heteroatoms as ring atoms in the aromatic ring and the remainder of the ring atoms being carbon atoms. Suitable heteroatoms include oxygen, sulfur, nitrogen, and selenium. Suitable heteroaryl groups include furanyl, thienyl, pyridyl, pyrrolyl, N-lower alkyl pyrrolyl, pyridyl-N-oxide, pyrimidyl, pyrazinyl, imidazolyl, and the like, all optionally substituted.

The phrase “carbocyclic ring” refers to a saturated or unsaturated monocyclic or bicyclic ring in which all atoms of all rings are carbon. Thus, the term includes cycloalkyl and carbocyclic aryl rings.

The phrase “heterocyclic ring” refers to a saturated or unsaturated monocyclic or bicyclic ring having from 1 to 4 heteroatoms as ring atoms in the aromatic ring and the remainder of the ring atoms being carbon atoms. Thus, the term includes heterocycloalkyl and heteroaryl rings.

In certain embodiments, “optionally substituted” or “substituted” includes groups substituted by one or more substituents independently selected from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is independently optionally substituted with one or more substituents selected from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰, —OC(O)OR¹, —OC(O)N(R¹⁰⁰)₂, —NR¹⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂, C₁₋₆ alkyl, C₁₋₆ haloalkyl, C₂₋₆ alkenyl, and C₂₋₆ alkynyl, wherein R¹⁰⁰ at each occurrence is independently selected from hydrogen; and C₁₋₂₀ alkyl, C₂₋₂₀ alkenyl, C₂₋₂₀ alkynyl, C₃₋₁₂ carbocycle and 3- to 12-membered heterocycle, each of which may be optionally substituted by halogen, —CN, —NO₂, —OH and —OCH₃. In certain embodiments, the term “optionally substituted” or “substituted” includes groups substituted by one to four substituents, independently selected from lower alkyl, lower aryl, lower aralkyl, lower alicyclic, heterocycloalkyl, hydroxyl, lower alkoxy, lower aryloxy, perhaloalkoxy, aralkoxy, heteroaryl, heteroaryloxy, heteroarylalkyl, heteroaralkoxy, azido, amino, guanidino, amidino, halo, lower alkylthio, oxo, acylalkyl, carboxy esters, carboxyl,-carboxamido, nitro, acyloxy, aminoalkyl, alkylaminoaryl, alkylaryl, alkylaminoalkyl, alkoxyaryl, arylamino, aralkylamino, phosphono, sulfonyl, -carboxamidoalkylaryl, -carboxamidoaryl, hydroxyalkyl, haloalkyl, alkylaminoalkylcarboxy-, aminocarboxamidoalkyl-, cyano, lower alkoxyl, lower perhaloalkyl, and arylalkyloxyalkyl.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

In some embodiments, the compounds described by the presently disclosed subject matter contain a linking group. As used herein, the term “linking group” comprises a chemical moiety which is bonded to two or more other chemical moieties to form a stable structure. In certain embodiments, the linking group, e.g., methylene, ethylene, links a moiety, e.g., an aryl or heteroaryl group, to the remainder of the structure.

“Alkylene” refers to a straight or branched bivalent aliphatic hydrocarbon group having from 1 to 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group can be optionally substituted with one or more substituents. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to 20, e.g., 0, 1, 2, 3, 4, 5, 6, 7, 8, 9, 10, 11, 12, 13, 14, 15, 16, 17, 18, 19, or 20, and R is hydrogen or lower alkyl. In certain embodiments, an alkylene group has from 1 to 6 carbon atoms such as from 1 to 3 carbon atoms.

The term “alkenylene” denotes a straight or branched bivalent aliphatic hydrocarbon group having from 2 to 20 carbon atoms with at least one carbon-carbon double bond. The alkenylene group can be optionally substituted with one or more substituents. Representative alkenylene groups include, but are not limited to, ethenylene, propenylene, 1- or 2-butenylene, 1-, or 2-pentylene, and the like.

As used herein, the term “acyl” refers to an group represented by R—C(═O)—, wherein R is, for example, an alkyl or an aryl group as defined herein. Specific examples of acyl groups include acetyl and benzoyl.

“Alkoxyl” refers to an alkyl-O-group wherein alkyl is as previously described. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

“Aryloxyl” refers to an aryl-O-group wherein the aryl group is as previously described, including a substituted aryl. The term “aryloxyl” as used herein can refer to optionally substituted phenyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O-group wherein the aralkyl group is as previously described. An exemplary aralkyloxyl group is benzyloxyl.

“Dialkylamino” refers to an —NRR′ group wherein each of R and R′ is independently selected from optionally substituted alkyl groups as previously described. Exemplary alkylamino groups include ethylmethylamino, dimethylamino, and diethylamino.

“Alkoxycarbonyl” refers to an alkoxyl-C(O)— group. Exemplary alkoxycarbonyl groups include methoxycarbonyl, ethoxycarbonyl, butyloxycarbonyl, and t-butyloxycarbonyl.

“Aryloxycarbonyl” refers to an aryloxyl —CO— group. Exemplary aryloxycarbonyl groups include phenoxycarbonyl and naphthoxycarbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group wherein one of R and R′ is hydrogen and the other of R and R′ is optionally substituted alkyl as previously described.

“Dialkylcarbamoyl” refers to a R′RN—CO— group wherein each of R and R′ is independently optionally substituted alkyl as previously described.

“Acyloxyl” refers to an acyl-O— group wherein acyl is as previously described.

“Acylamino” refers to an acyl-NH— group wherein acyl is as previously described.

The term “amino” refers to the —NH₂ group and amino may be optionally substituted.

The term “carbonyl” refers to the —C(O)— group.

The term “carboxyl” refers to the —COOH group.

The term “cyano” refers to the —CN group.

The terms “halo”, “halide”, or “halogen” as used herein refer to fluoro, chloro, bromo, and iodo groups.

The term “hydroxyl” refers to the —OH group.

The term “hydroxyalkyl” refers to an alkyl group substituted with one or more —OH substituents.

The term “haloalkyl” refers to an alkyl group with one or more independently selected halogen substituents.

The term “mercapto” refers to the —SH group.

The term “oxo” refers to ═O.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term “treatment” as used herein covers any treatment of a disease and/or condition in an animal or mammal, particularly a human, and includes: (i) preventing a disease, disorder and/or condition and/or symptoms from occurring in a person which can be predisposed to the disease, disorder and/or condition, or at risk for being exposed to an agent that can cause the disease, disorder, and/or condition and/or symptoms; but, has not yet been diagnosed as having it; (ii) inhibiting the disease, disorder and/or condition, and/or symptoms i.e., arresting its development; and (iii) relieving the disease, disorder and/or condition, and/or symptoms i.e., causing regression of the disease, disorder and/or condition. iv) the augmentation of a mechanism, such as modulating p75NTR signaling, that can lead to reduced symptoms and improved function.

The terms “modulate” and “modulation” as used herein is used in the common manner of the field as to regulate or adjust to a certain degree.

The term “derivative” as used herein refers to a compound chemically modified so as to differentiate it from a parent compound. Such chemical modifications can include, for example, replacement of hydrogen by an alkyl, acyl, or amino group. A derivative compound can be modified by, for example, glycosylation, pegylation, or any similar process that retains at least one biological or immunological function of the compound from which it was derived.

The term “hydrophilicity” is used in the common manner of the field as having an affinity for water; readily absorbing and/or dissolving in water.

The term “lipophilicity” is used in the common manner of the field as having an affinity for, tending to combine with, or capable of dissolving in lipids.

The term “amphipathicity”, as used herein, describes a structure having discrete hydrophobic and hydrophilic regions. Thus, one portion of the structure interacts favorably with aqueous and other polar media, while another portion of the structure interacts favorably with non-polar media.

The term “solubility” as used herein, describes the maximum amount of solute that will dissolve in a given amount of solvent at a specified temperature.

The term “bioavailability” as used herein refers to the systemic availability, blood/plasma levels, of a given amount of compound administered to a subject. The term further encompasses the rate and extent of absorption of compound that reaches the site of action.

As used herein, “solvate” means a complex formed by the combination of solvent molecules with molecules or ions of the compound or salt of the disclosure. Examples of hydrate include, but are not limited to, hemihydrate, monohydrate, dihydrate, trihydrate, hexahydrate, etc. Solvates, including hydrates, may be found in stoichiometric ratios, for example, with two, three, four salt molecules of the disclosure per solvate or per hydrate molecule. Solvents used for crystallization, such as alcohols, especially methanol and ethanol; aldehydes; ketones, especially acetone; esters, e.g. ethyl acetate; may be embedded in the crystal grating.

The term “prodrug” as used herein refers to any compound that when administered to a biological system generates the drug substance (a biologically active compound) in steps involving, for example, spontaneous chemical reaction(s), enzyme catalyzed chemical reaction(s), or both.

Where the compounds of the present invention have at least one asymmetric center, they may accordingly exist as enantiomers. Where the compounds possess two or more asymmetric centers, they may additionally exist as diastereoisomers. It is to be understood that all such stereoisomers and mixtures thereof in any proportion are encompassed within the scope of the present invention. Where the compounds possess geometrical isomers, all such isomers and mixtures thereof in any proportion are encompassed within the scope of the present invention. Where so indicated in the claims herein, if a single enantiomer of the potentially optically active heterocyclic compounds disclosed is desired, for either health or efficacy reasons, preferably it is present in an enantiomeric excess of at least about 80%, or at least about 90%, or at least about 95%, or at least about 98%, or at least about 99%, or at least about 99.5%.

### Compounds of the Disclosure

In one aspect, the disclosure provides compounds and compositions and methods of use thereof. The compound and compositions of the disclosure may be used in the treatment and prevention of an alcohol use disorder and associated diseases. In one aspect, a compound of the disclosure is represented by Formula III:

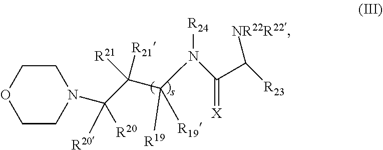

or a salt thereof, wherein:

X is CH₂, NH, O or S;

s is 0, 1, 2, 3 or 4;

each of R¹⁹, R^{19′}, R²⁰, R^{20′}, R²¹, R^{21′}, R²², R^{22′} and R²⁴ is independently selected at each occurrence from hydrogen and optionally substituted alkyl; or

R²⁰ and R^{20′} taken together form ═O, ═S, or ═CH₂; or

R²⁰ and R²¹ taken together with the atoms to which they are attached form an optionally substituted cycloalkyl; or

R²⁰ and R²¹ taken together with the atoms to which they are attached form an optionally substituted aryl; or

R¹⁹ and R²⁰ taken together with the atoms to which they are attached form an optionally substituted cycloalkyl; or

R¹⁹ and R²⁰ taken together with the atoms to which they are attached form an optionally substituted aryl; and

R²³ is hydrogen, optionally substituted alkyl, optionally substituted cycloalkyl or optionally substituted aryl.

In certain embodiments, for a compound or salt of Formula III, s is 0, 1 or 2. In an exemplary embodiment, s is 0.

In certain embodiments, for a compound or salt of Formula III, X is NH, O or S. In an exemplary embodiment, X is O.

For a compound or salt of Formula III, when R¹⁹, R^{19′}, R²⁰, R^{20′}, R²¹, R^{21′}, R²², R^{22′} or R²⁴ is optionally substituted, the optional substituents may be independently selected at each occurrence from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is independently optionally substituted with one or more substituents selected from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂, C₁₋₆ alkyl, C₁₋₆ haloalkyl, C₂₋₆ alkenyl, and C₂₋₆ alkynyl, wherein R¹⁰⁰ at each occurrence is independently selected from hydrogen; and C₁₋₂₀ alkyl, C₂₋₂₀ alkenyl, C₂₋₂₀ alkynyl, C₃₋₁₂ carbocycle and 3- to 12-membered heterocycle, each of which may be optionally substituted by halogen, —CN, —NO₂, —OH and —OCH₃.

For a compound or salt of Formula III, when R¹⁹, R^{19′}, R²⁰, R^{20′}, R²¹, R^{21′}, R²², R^{22′} or R²⁴ is optionally substituted, the optional substituents may be independently selected at each occurrence from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂; C₁₋₁₀ alkyl optionally substituted at each occurrence with one or more substituents selected from halogen, —NO₂, —CN, —OR¹⁰⁰, —SR¹⁰⁰, and —N(R¹⁰⁰)₂; and C₃₋₁₂ carbocycle and 3- to 12-membered heterocycle, wherein each C₃₋₁₂ carbocycle and 3- to 12-membered heterocycle is independently optionally substituted with one or more substituents selected from halogen, —NO₂, —CN, —OR¹⁰⁰, —SR¹⁰⁰, —N(R¹⁰⁰)₂, C₁₋₆ alkyl, and C₁₋₆ haloalkyl, wherein R¹⁰⁰ at each occurrence is independently selected from hydrogen; and C₁₋₂₀ alkyl, C₂₋₂₀ alkenyl, C₂₋₂₀ alkynyl, C₃₋₁₂ carbocycle and 3- to 12-membered heterocycle, each of which may be optionally substituted by halogen, —CN, —NO₂, —OH and —OCH₃.

In some embodiments, for a compound or salt of Formula III, s is selected from 1, 2, 3 or 4 and R¹⁹ and R^{19′} are independently selected at each occurrence from hydrogen and optionally substituted C₁-C₆ alkyl. In certain embodiments, s is selected from 1 or 2 and R¹⁹ and R^{19′} are independently selected at each occurrence from hydrogen and optionally substituted C₁-C₃ alkyl.

In certain embodiments, for a compound or salt of Formula III, R²⁰ and R^{20′} are independently selected from hydrogen and optionally substituted C₁-C₆ alkyl. In certain embodiments, R²⁰ and R^{20′} are independently selected from hydrogen and optionally substituted C₁-C₃ alkyl. In certain embodiments, R²⁰ and R^{20′} are each hydrogen.

In certain embodiments, for a compound or salt of Formula III, R²¹ and R^{21′} are independently selected from hydrogen and optionally substituted C₁-C₆ alkyl. In certain embodiments, R²¹ and R^{21′} are independently selected from hydrogen and optionally substituted C₁-C₃ alkyl. In certain embodiments, R²¹ and R^{21′} are each hydrogen.

In certain embodiments, for a compound or salt of Formula III, R²² and R^{22′} are independently selected from hydrogen and optionally substituted C₁-C₆ alkyl. In certain embodiments, R²² and R^{22′} are independently selected from hydrogen and optionally substituted C₁-C₃ alkyl. In certain embodiments, R²² and R^{22′} are each hydrogen.

In certain embodiments, for a compound or salt of Formula III, R²³ is selected from hydrogen, optionally substituted C₁-C₆ alkyl, optionally substituted cycloalkyl and optionally substituted aryl. In certain embodiments, R²³ is selected from optionally substituted C₁-C₆ alkyl, such as optionally substituted C₂-C₅ alkyl, such as optionally substituted C₃-C₅ alkyl, such as optionally substituted C₄ alkyl. In certain embodiments, R²³ is C₃-C₅ alkyl, such as C₄ alkyl. R²³ may be represented by the following structure:

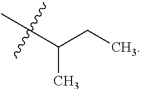 [SMILES: *C(C)CC]

In certain embodiments, for a compound or salt of Formula III, R² is hydrogen or optionally substituted C₁-C₆ alkyl alkyl. In certain embodiments, R²⁴ is selected from hydrogen and optionally substituted C₁-C₃ alkyl. In certain embodiments, R²⁴ is hydrogen.

In certain embodiments, for a compound or salt of Formula III, s is 0, X is O or S, R²⁰ and R^{20′} are independently selected from hydrogen and optionally substituted C₁-C₃ alkyl, R²¹ and R^{21′} are independently selected from hydrogen and optionally substituted C₁-C₃ alkyl, R²² and R^{22′} are independently selected from hydrogen and optionally substituted C₁-C₃ alkyl, R²³ is optionally substituted C₂-C₅ alkyl, and R²⁴ is selected from hydrogen and optionally substituted C₁-C₃ alkyl.

In certain embodiments, for a compound or salt of Formula III, s is 0, X is O or S, R²⁰ and R^{20′} are each hydrogen, R²¹ and R^{21′} are each hydrogen, R²² and R^{22′} are independently selected from hydrogen and optionally substituted C₁-C₃ alkyl, R²³ is optionally substituted C₂-C₅ alkyl, and R²⁴ is selected from hydrogen and optionally substituted C₁-C₃ alkyl.

In certain embodiments, for a compound or salt of Formula III, s is 0, X is O, R²⁰ and R^{20′} are independently selected from hydrogen and optionally substituted C₁-C₃ alkyl, R²¹ and R^{21′} are independently selected from hydrogen and optionally substituted C₁-C₃ alkyl, R²² and R^{22′} are each hydrogen, R²³ is optionally substituted C₄ alkyl, and R²⁴ is hydrogen.

In certain embodiments, the compound of Formula III is represented by the structural formula:

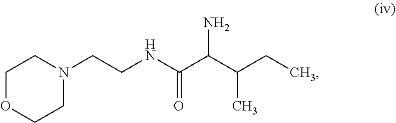 [SMILES: CCC(C)C(N)C(=O)NCCN1CCOCC1]

or a salt thereof, e.g., a sulfuric acid addition salt thereof. In certain embodiments, the compound of Formula III is represented by the structural formula:

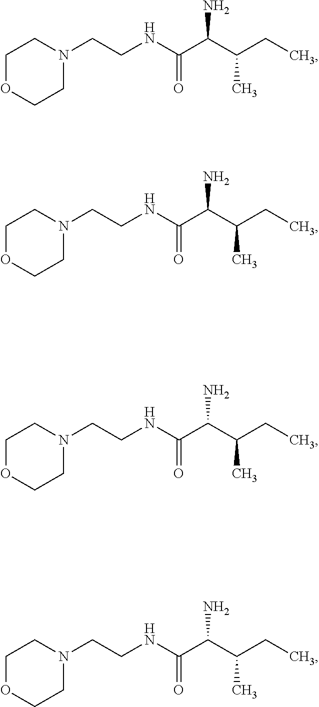 [SMILES: CC[C@H](C)[C@H](N)C(=O)NCCN1CCOCC1 ; CC[C@@H](C)[C@H](N)C(=O)NCCN1CCOCC1 ; CC[C@@H](C)[C@@H](N)C(=O)NCCN1CCOCC1 ; CC[C@H](C)[C@@H](N)C(=O)NCCN1CCOCC1]

or a salt of any one thereof, e.g., a sulfuric acid addition salt thereof of any one thereof. In certain embodiments, the salt of the compound of Formula III, is represented by Formula X:

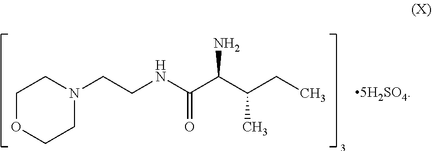

In certain embodiments, the compound of Formula III is represented by the formula:

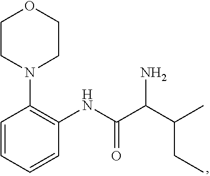 [SMILES: CCC(C)C(N)C(=O)Nc1ccccc1N1CCOCC1]

or a salt thereof.

In certain embodiments, for a compound of Formula III, s is 2; X is O; R¹⁹ and R²⁰ taken together with the atoms to which they are attached form an optionally substituted cycloalkyl; or R¹⁹ and R²⁰ taken together with the atoms to which they are attached form an optionally substituted aryl. In certain embodiments, the compound of Formula III is represented by the formula:

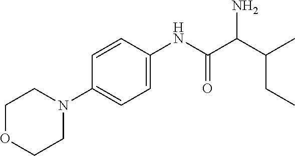 [SMILES: CCC(C)C(N)C(=O)Nc1ccc(N2CCOCC2)cc1]

or a salt thereof.

In certain embodiments, the compound of Formula III is not selected from:

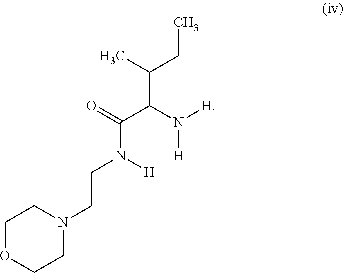

In certain embodiments, the disclosure provides a compound selected from:

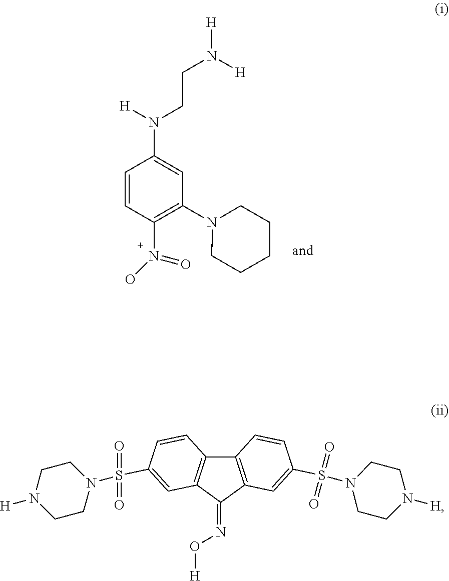

or a salt of either one thereof.

In certain embodiments, the present application discloses a compound selected from a compound of Formula A or Formula B:

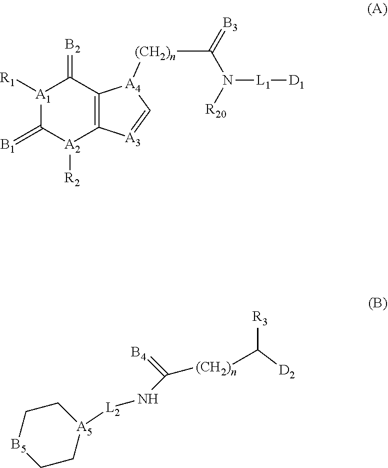

wherein:
n is an integer from 0 to 8;
L₁ and L₂ are a linking group selected from the group consisting of alkylene, substituted alkylene, cycloalkyl, substituted cycloalkyl, cycloalkene, substituted cycloalkene, aryl, substituted aryl, alkenylene, and substituted alkenylene;
R₁, R₂, and R₃ are each independently selected from the group consisting of H, alkyl, substituted alkyl, cycloalkyl, halo, cyano, nitro, mercapto, hydroxyl, alkoxyl, aryl, aryloxyl, substituted aryl, and aralkyloxyl;
A₁, A₂, A₃, A₄, and A₅ are each independently selected from the group consisting of N and C;
B₁, B₂, B₃, B₄, and B₅ are each independently selected from the group consisting of O, S, and NR₄, wherein R₄ is selected from the group consisting of H, alkyl, substituted alkyl, cycloalkyl, aryl, and substituted aryl; and
D₁ and D₂ are selected from the group consisting of:

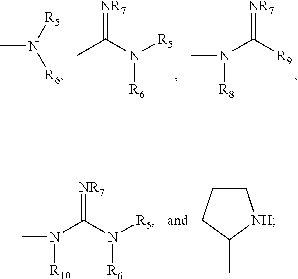

wherein:
each R₅, R₆, R₈, R₉, R₁₀, and R₂₀ is independently selected from the group consisting of H, alkyl, substituted alkyl, cycloalkyl, aryl, substituted aryl, aralkyl, hydroxyalkyl, hydroxycycloalkyl, alkoxycycloalkyl, aminoalkyl, acyloxyl, alkylaminoalkyl, and alkoxycarbonyl;
each R₇ is independently selected from the group consisting of H, hydroxyl, alkyl, substituted alkyl, aryl, substituted aryl, acyloxyl, and alkoxyl; or
R₇ and R₅ or R₇ and R₉ together represent a C₂ to C₁₀ alkyl, C₂ to C₁₀ hydroxyalkyl, or C₂ to C₁₀ alkene; or a pharmaceutically acceptable salt thereof.

In some embodiments, for a compound of Formula A or B, L₁ and L₂ are each independently —(CH₂)ₘ—, wherein m is an integer from 1 to 8.

In certain embodiments, the compound of the disclosure is a compound of Formula A:

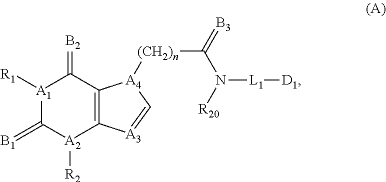

or a salt thereof, wherein:
n is an integer from 0 to 4;
L₁ is a linking group selected from alkylene, —C(═O)—, cycloalkyl, cycloalkyl, cycloalkenyl, aryl, heteroaryl, and alkenylenyl;
R₁ and R₂ are each independently selected from H, alkyl, cycloalkyl, halo, cyano, nitro, mercapto, hydroxyl, alkoxyl, aryl, aryloxyl, heteroaryl, and aralkyloxyl;
A₁, A₂, A₃, and A₄ are independently selected from N and C;
B₁, B₂, and B₃, are independently selected from O, S, and NR₄, wherein R₄ is selected from H, alkyl, substituted alkyl, cycloalkyl, aryl, and substituted aryl; and D₁ is selected from:

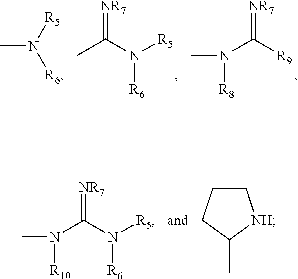

wherein each R₅, R₆, R₈, R₉, R₁₀, and R₂₀ is independently selected from the group consisting of H, alkyl, substituted alkyl, cycloalkyl, aryl, substituted aryl, aralkyl, hydroxyalkyl, hydroxycycloalkyl, alkoxycycloalkyl, aminoalkyl, acyloxyl, alkylaminoalkyl, and alkoxycarbonyl; and each R₇ is independently selected from H, hydroxyl, alkyl, substituted alkyl, aryl, substituted aryl, acyloxyl, and alkoxyl.

In certain embodiments, the compound of Formula A is represented by the structure:

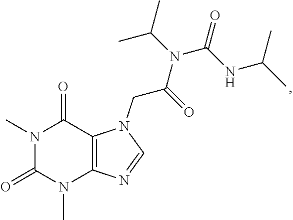 [SMILES: CC(C)NC(=O)N(C(=O)Cn1cnc2c1c(=O)n(C)c(=O)n2C)C(C)C]

or a salt thereof.

In some embodiments, the compound of Formula A has the following structure:

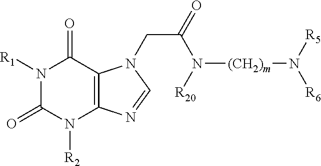

wherein:
m is an integer from 1 to 8;
R₁, R₂ and R₂₀ are each independently selected from H, alkyl, substituted alkyl, cycloalkyl, aryl, aryloxyl, substituted aryl, and aralkyloxyl; and
R₅ and R₆ are each independently selected from H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl.

In certain embodiments, the compound of Formula A is represented by the structure:

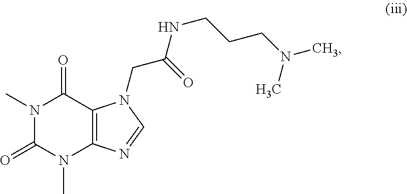 [SMILES: CN(C)CCCNC(=O)Cn1cnc2c1c(=O)n(C)c(=O)n2C]

or a salt thereof. In some embodiments, the compound of Formula A is not:

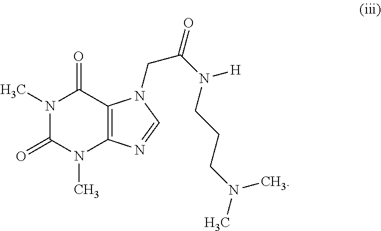 [SMILES: CN(C)CCCNC(=O)Cn1cnc2c1c(=O)n(C)c(=O)n2C]

In some embodiments, the compound of Formula B has the following structure:

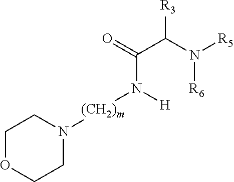

wherein:
m is an integer from 1 to 8;
R₃ is selected from H, alkyl, substituted alkyl, cycloalkyl, halo, hydroxyl, alkoxyl, aryl, aryloxyl, substituted aryl, and aralkyloxyl; and
R₅ and R₆ are each independently selected from H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl.

In some embodiments, the compound has the formula:

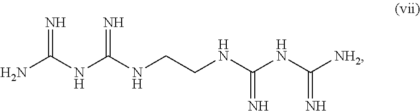 [SMILES: N=C(N)NC(=N)NCCNC(=N)NC(=N)N]

or a salt thereof.

In another aspect, the present application discloses a compound of Formula II:

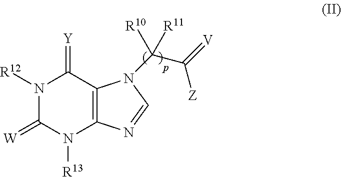

or a pharmaceutically acceptable salt, ester, prodrug or solvate thereof, wherein p is 0, 1, 2, 3, 4, 5, or 6; each of Y, V, and W is independently ═CH₂, ═NH, ═O or ═S; each of R¹⁰ and R″ is independently hydrogen or optionally substituted alkyl; each of R¹² and R¹³ is independently hydrogen, —NR^{a}R^{b}, —OH, —C(═O)OR^{a}, —C(═O)NHR^{a}, —NHC(═O)R^{a}, —NHS(═O)₂R^{a}, or optionally substituted alkyl; each of R^{a} and R^{b} is independently hydrogen or optionally substituted alkyl; and Z is heterocycloalkyl or heteroaryl wherein each heterocycloalkyl or heteroaryl is bound via a heteroatom and is optionally substituted. Another aspect is a compound of Formula II wherein p is 1, 2, 3, 4, or 5; each of Y, V, and W is independently ═O or ═S; each of R¹⁰ and R¹¹ is independently hydrogen, optionally substituted alkyl, optionally substituted alkenyl or optionally substituted alkynyl; each of R¹² and R¹³ is independently hydrogen, —NR^{a}R^{b}, —OH, optionally substituted alky, optionally substituted alkenyl or optionally substituted alkynyl; each of R^{a} and R^{b} is independently hydrogen or optionally substituted alkyl; and Z is heterocycloalkyl or heteroaryl wherein each heterocycloalkyl or heteroaryl is bound via a heteroatom and is optionally substituted. In one embodiment of any of the disclosed aspects, p is 1, 2 or 3; each of Y, V, and W is O; and each of R¹² and R¹³ is independently hydrogen or optionally substituted C₁-C₄ alkyl; and Z is an optionally substituted nitrogen-bound heterocycloalkyl. In another embodiment, p is 1; each of R¹⁰ and R¹¹ is hydrogen; and each of R¹² and R¹³ is independently C₁-C₄ alkyl.

In another embodiment, the compound has the structure of Formula IIA:

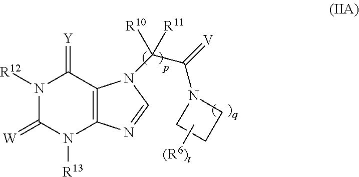

or a pharmaceutically acceptable salt, ester, prodrug or solvate thereof, wherein p is 0, 1, 2, 3, 4, 5, or 6; q is 1, 2, 3, or 4; t is 0, 1, 2, or 3; each of Y, V, and W is independently O or S; each of R¹⁰ and R¹¹ is independently hydrogen or optionally substituted alkyl; each of R¹² and R¹³ is independently hydrogen, —NR^{a}R^{b}, —OH, —C(═O)OR^{a}, —C(═O)NHR^{a}, —NHC(═O)R^{a}, —NHS(═O)₂R^{a}, or optionally substituted alkyl; each of R⁶ is independently —NR^{a}R^{b}, —OH, —C(═O)OR^{a}, —C(═O)NHR^{a}, —NHC(═O)R^{a}, —NHS(═O)₂R^{a}, or optionally substituted alkyl; and each of R^{a} and R^{b} is independently hydrogen or optionally substituted alkyl. In one embodiment, q is 1, 2, 3, or 4; t is 0, 1, 2, or 3; each of Y, V, and W is independently O or S; and each of R⁶ is independently —NR^{a}R^{b}, —OH, —C(═O)OR^{a}, —C(═O)NHR^{a}, —NHC(═O)R^{a}, —NHS(═O)₂R^{a}, or optionally substituted alkyl. In yet another aspect, the compound has the structure of Formula IIA wherein p is 1, 2, 3, 4, or 5; q is 2 or 3; t is 0, 1, 2, or 3; each of Y, V, and W is independently O or S; each of R¹⁰ and R¹¹ is independently hydrogen, optionally substituted alkyl, optionally substituted alkenyl, or optionally substituted alkynyl; each of R⁶ is independently —NR^{a}R^{b}, —OH, or optionally substituted alkyl; and each of R^{a} and R^{b} is independently hydrogen or optionally substituted alkyl. In another embodiment of any of the disclosed aspects, each of Y, V, and W is O; q is 1; each of R¹⁰ and R¹¹ is independently hydrogen or C₁-C₄ alkyl; and each of R¹² and R¹³ is independently C₁-C₄ alkyl. In yet another embodiment, each of R¹⁰ and R¹¹ is independently hydrogen; each of R¹² and R¹³ is independently —Me; and each of R⁶, R^{6′}, R⁷, R^{7′}, R⁸, R^{8′}, R⁹, and R^{9′} is independently hydrogen, —NR^{a}R^{b}, —OH, or optionally substituted alkyl. In still a further variation of any of the disclosed embodiments, each of R¹⁰ and R¹¹ is independently —H; each of R¹² and R¹³ is independently —Me; q is 2; and each of R⁶, R^{6′}, R⁷, R^{7′}, R⁸, R^{8′}, R⁹, and R^{9′} is independently hydrogen, —NR^{a}R^{b}, —OH, or optionally substituted alkyl. In another further variation of any of the disclosed embodiments, each of R⁶, R^{6′}, R⁷, R^{7′}, R⁸, R^{8′}, and R⁹ is hydrogen; and R^{9′} is —N(CH₃)₂.

In another embodiment, the compound has the structure of Formula IIB:

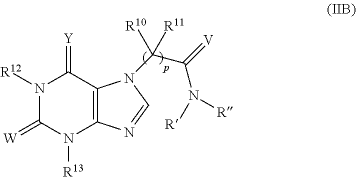

or a pharmaceutically acceptable salt, ester, prodrug or solvate thereof, wherein p is 0, 1, 2, 3, 4, 5, or 6; each of Y, V, and W is independently O or S; each of R¹⁰ and R¹¹ is independently hydrogen or optionally substituted alkyl; each of R¹² and R¹³ is independently hydrogen, —NR^{a}R^{b}, —OH, —C(═O)OR^{a}, —C(═O)NHR^{a}, —NHC(═O)R^{a}, —NHS(═O)₂R^{a}, or optionally substituted alkyl; and R and R¹¹ taken together with the nitrogen to which they are attached form an optionally substituted heteroaryl. In yet another aspect, the compound has the structure of Formula IIB wherein p is 1, 2, 3, 4, or 5; each of Y, V, and W is independently O or S; each of R¹⁰ and R¹¹ is independently hydrogen, optionally substituted alkyl, optionally substituted alkenyl, or optionally substituted alkynyl; R and R taken together with the nitrogen to which they are attached form a an optionally substituted pyridyl, an optionally substituted pyrrolyl, an optionally substituted pyrimidyl or an optionally substituted pyrazinyl. In another embodiment of any of the disclosed aspects, each of Y, V, and W is O; each of R¹⁰ and R¹¹ is independently hydrogen or C₁-C₄ alkyl; and each of R¹² and R¹³ is independently C₁-C₄ alkyl. In yet another embodiment, each of R¹⁰ and R¹¹ is independently hydrogen; each of R¹² and R¹³ is independently —Me. In still a further variation of any of the disclosed embodiments, each of R¹⁰ and R¹¹ is independently —H; each of R¹² and R¹³ is independently —Me; q is 2; and R′ and R″ taken together with the nitrogen to which they are attached form an optionally substituted pyrrolyl. In another further variation, the compound has the structural formula:

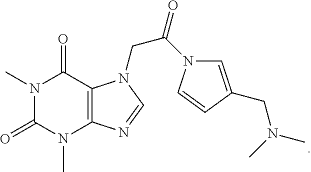 [SMILES: CN(C)Cc1ccn(C(=O)Cn2cnc3c2c(=O)n(C)c(=O)n3C)c1]

In still another aspect, the present application discloses a compound of Formula IV:

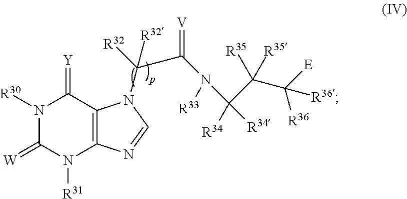

or a pharmaceutically acceptable salt, ester, prodrug or solvate thereof, wherein p is 1, 2, 3, 4, 5, or 6; each of Y, V, and W is independently CH₂, NH, O or S; each of R³⁰, R³¹, R³², R^{32′} R³³, R³⁴, R^{34′}, R³⁵, R^{35′}, R³⁶, and R^{36′} is independently absent, hydrogen or optionally substituted alkyl; or R³⁴ and R³⁶ taken together with the atoms to which they are attached form an optionally substituted carbocyclic ring; E is —CHR^{c}R^{d}, —NR^{c}R^{d}, —OR^{c}, and —SR; and each of R^{c} and R^{d} is independently hydrogen or optionally substituted alkyl; or R^{c} and R^{d} taken together with the nitrogen atom to which they are attached form an optionally substituted heterocyclic ring or R^{c} and R^{d} taken together with the carbon atom to which they are attached form an optionally substituted carbocyclic ring. In one embodiment of any of the disclosed aspects or variations, the compound of Formula IV is not N-(3-(diethylamino)propyl)-2-(4,6-dimethyl-5,7-dioxo-4,5,6,7-tetrahydro-1H-benzo[d]imidazol-1-yl)acetamide. In one variation, p is 1, 2, or 3; each of Y, V, and W is O or S; each of R³⁰ and R³¹ is independently optionally substituted C₁-C₄ alkyl; each of R³², R^{32′} R³³, R³⁴, R^{34′}, R³⁵, R^{35′}, R³⁶, and R^{36′} is independently hydrogen or optionally substituted C₁-C₄ alkyl; and E is —OR^{c}, —SR^{c}, or —NR^{c}R^{d} wherein R^{c} and R^{d} taken together with the nitrogen atom to which they are attached form an optionally substituted heterocycloalkyl.

In one aspect, the compound has the structure of Formula IV wherein p is 1, 2, 3, or 4; each of Y, V, and W is independently O or S; each of R³⁰, R³¹, R³², R^{32′} R³³, R³⁴, R^{34′}, —R³⁵, R^{35′}, R³⁶, and R^{36′} is independently absent, hydrogen, optionally substituted alkyl, optionally substituted alkenyl or optionally substituted alkynyl; or R³⁴ and R³⁶ taken together with the atoms to which they are attached form an optionally substituted carbocyclic ring; E is —CHR^{c}R^{d}, —NR^{c}R^{d}, —OR^{c}, or —SR; and each of R^{c} and R^{d} is independently hydrogen, optionally substituted alkyl, optionally substituted alkenyl or optionally substituted alkynyl; or R^{c} and R^{d} taken together with the nitrogen atom to which they are attached form an optionally substituted heterocyclic ring or R^{c} and R^{d} taken together with the carbon atom to which they are attached form an optionally substituted carbocyclic ring. In one embodiment of any of the disclosed aspects, p is 1, 2, or 3; each of Y, V, and W is O or S; E is —OR^{c} or —SR^{c}; each of R³², R^{32′} R³³, R^{34′}, R³⁵, R^{35′,} and R^{36′} is independently hydrogen; and each of R³⁰ and R³¹ is independently optionally substituted C₁-C₄ alkyl. In another embodiment, p is 1, 2, or 3; each of Y, V, and W is O or S; E is NR^{c}R^{d} and R^{c} and R^{d} taken together with the nitrogen atom to which they are attached form an optionally substituted heterocycloalkyl; each of R³⁰ and R³¹ is independently optionally substituted C₁-C₄ alkyl; and each of R³², R^{32′} R³³, R³⁴, R^{34′}, R³⁵, R^{35′}, R³⁶, and R^{36′} is independently hydrogen or optionally substituted C₁-C₄ alkyl. In yet another embodiment, p is 1; each of Y, V, and W is O; each of R³⁰ and R³¹ is independently —CH₃; R³³ is hydrogen; and each of R³², R^{32′} R³⁴, R^{34′}, R³, R^{35′}, R³⁶, and R^{36′} is independently hydrogen or C₁-C₄ alkyl. In one variation of any of the disclosed embodiments, E is —NR^{c}R^{d} and each of R^{c} and R^{d} is independently hydrogen or optionally substituted alkyl. In another embodiment, R³⁴ and R³⁶ taken together with the atoms to which they are attached form an optionally substituted cycloalkyl; or R³⁴ and R³⁶ taken together with the atoms to which they are attached form an optionally substituted carbocyclic aryl. In another embodiment, the compound has the structural formula:

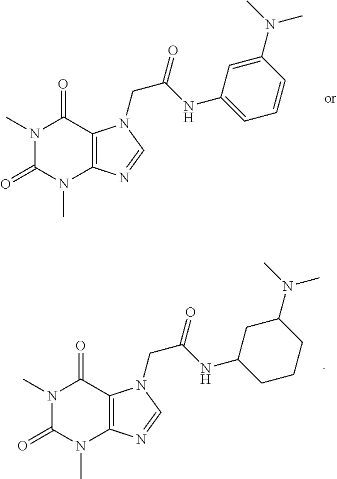 [SMILES: CN(C)c1cccc(NC(=O)Cn2cnc3c2c(=O)n(C)c(=O)n3C)c1 ; CN(C)C1CCCC(NC(=O)Cn2cnc3c2c(=O)n(C)c(=O)n3C)C1]

In another variation of any of the disclosed embodiments, E is —NR^{c}R^{d} and R^{c} and R^{d} taken together with the nitrogen atom to which they are attached form an optionally substituted heterocycloalkyl. In one variation, the compound has a structural formula selected from:

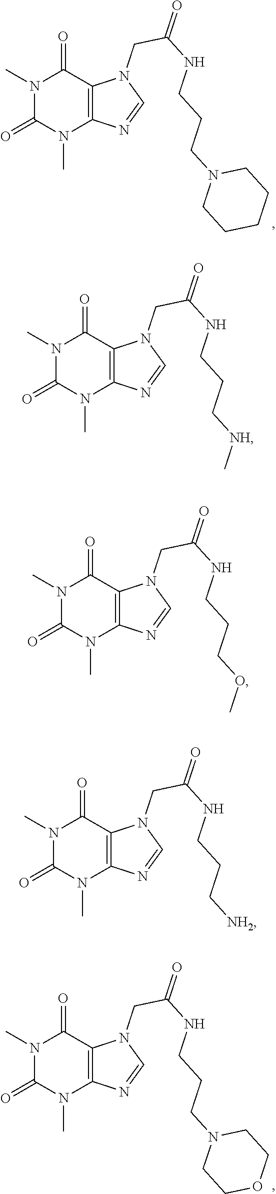

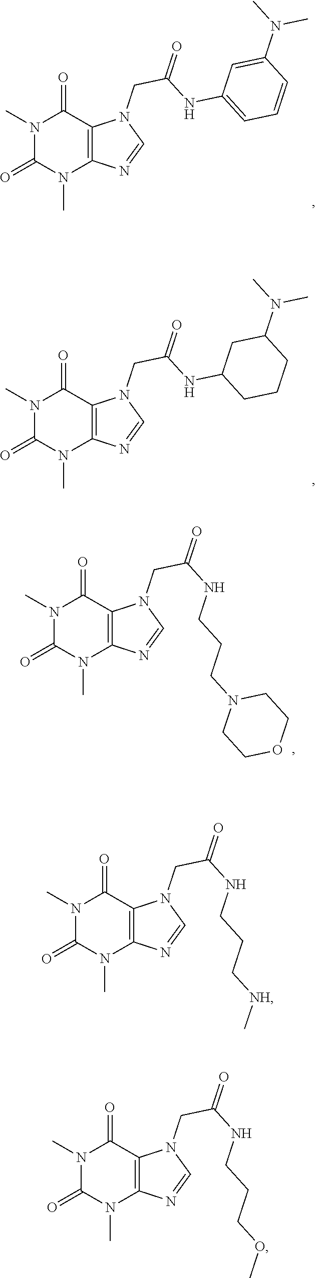

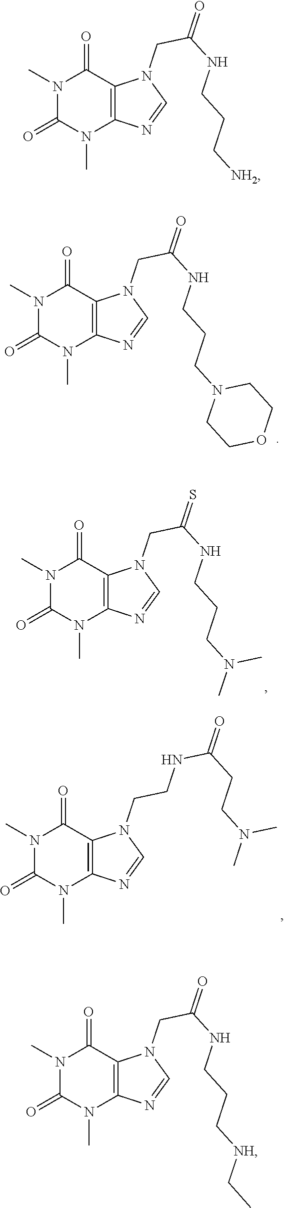

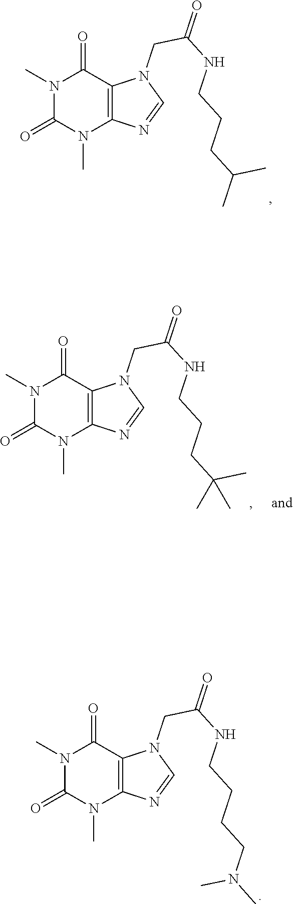

In another aspect, the present application provides a compound of Formula IVA:

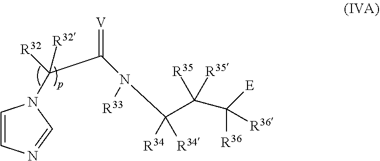

or a pharmaceutically acceptable salt, ester, prodrug or solvate thereof, wherein p is 1, 2, 3, 4, 5, or 6; V is CH₂, NH, O or S; each of R³², R^{32′} R³³, R³⁴, R^{34′}, R³⁵, R^{35′}, R³⁶, and R^{36′} is independently absent, hydrogen or optionally substituted alkyl; or R³⁴ and R³⁶ taken together with the atoms to which they are attached form an optionally substituted carbocyclic ring; E is —CHR^{c}R^{d}, —N^{c}R^{d}, —OR^{c}, and —SR; and each of R^{c} and R^{d} is independently hydrogen or optionally substituted alkyl; or R^{c} and R^{d} taken together with the nitrogen atom to which they are attached form an optionally substituted heterocyclic ring or R^{c} and R^{d} taken together with the carbon atom to which they are attached form an optionally substituted carbocyclic ring. In one aspect, the compound has the structure of Formula IVA wherein p is 1, 2, 3, or 4; V is O or S; each of R³², R^{32′} R³³, R³⁴, R^{34′}, R³⁵, R^{35′}, R³⁶, and R^{36′} is independently absent, hydrogen, optionally substituted alkyl, optionally substituted alkenyl or optionally substituted alkynyl; or R³⁴ and R³⁶ taken together with the atoms to which they are attached form an optionally substituted carbocyclic ring; E is —CHR^{c}R^{d}, —NR^{c}R^{d}, —OR^{c}, or —SR; and each of R^{c} and R^{d} is independently hydrogen, optionally substituted alkyl, optionally substituted alkenyl or optionally substituted alkynyl; or R^{c} and R^{d} taken together with the nitrogen atom to which they are attached form an optionally substituted heterocyclic ring or R^{c} and R^{d} taken together with the carbon atom to which they are attached form an optionally substituted carbocyclic ring. In one embodiment of any of the disclosed aspects, p is 1, 2, or 3; V is O or S; E is —OR^{c} or —SR; each of R³², R^{32′} R³³, R^{34′}, R³⁵, R^{35′,} and R^{36′} is independently hydrogen. In another embodiment, p is 1, 2, or 3; V is O or S; E is NR^{c}R^{d} and R^{o} and R^{d} taken together with the nitrogen atom to which they are attached form an optionally substituted heterocycloalkyl; and each of R³², R^{32′} R³³, R³⁴, R^{34′}, R³⁵, R^{35′}, R³⁶, and R^{36′} is independently hydrogen or optionally substituted C₁-C₄ alkyl. In yet another embodiment, p is 1; V is O; R³³ is hydrogen; and each of R³², R^{32′} R³⁴, R^{34′}, R³⁵, R^{35′}, R³⁶, and R^{36′} is independently hydrogen or C₁-C₄ alkyl. In one variation of any of the disclosed embodiments, E is —NR^{c}R^{d} and each of R^{c} and R^{d} is independently hydrogen or optionally substituted alkyl. In another embodiment, R³⁴ and R³⁶ taken together with the atoms to which they are attached form an optionally substituted cycloalkyl; or R³⁴ and R³⁶ taken together with the atoms to which they are attached form an optionally substituted carbocyclic aryl. In one embodiment, the compound has the structural formula

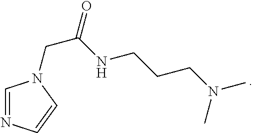

Since p75NTR is upregulated in various pathological states, compounds disclosed herein can also be linked to molecular markers that can be detected by imaging or other modalities. Such conjugates can be prepared according to synthetic methods known to those of skill in the art and applied in diagnostic strategies designed to detect such pathological states.

In another aspect, the present invention provides a compound selected from the group consisting of (2R,3R)-2-amino-3-methyl-N-(2-morpholinoethyl)-pentanamide; (2R,3 S)-2-amino-3-methyl-N-(2-morpholinoethyl)-pentanamide; and (2S,3R)-2-amino-3-methyl-N-(2-morpholinoethyl)-pentanamide; or a pharmaceutically acceptable salt, solvate, ester, or prodrug thereof. In one embodiment, the compound is in a purity of about 80% or more. In another embodiment, the compound is in a purity of about 85% or more. In another embodiment, the compound is in a purity of about 90% or more. In another embodiment, the compound is in a purity of about 95% or more. In another embodiment, the compound is in a purity of about 96% or more. In another embodiment, the compound is in a purity of about 97% or more. In another embodiment, the compound is in a purity of about 98% or more. In another embodiment, the compound is in a purity of about 99% or more. In another embodiment, the compound is in a purity of about 99.5% or more.

In another aspect, the present invention provides a mixture of two or more compounds selected from the group consisting of (2S,3 S)-2-amino-3-methyl-N-(2-morpholinoethyl)-pentanamide; (2R,3R)-2-amino-3-methyl-N-(2-morpholinoethyl)-pentanamide; (2R,3S)-2-amino-3-methyl-N-(2-morpholinoethyl)-pentanamide; and (2S,3R)-2-amino-3-methyl-N-(2-morpholinoethyl)-pentanamide; or a pharmaceutically acceptable salt, solvate, ester, or prodrug thereof, with the proviso that when the mixture consists of (2S,3 S)-2-amino-3-methyl-N-(2-morpholinoethyl)-pentanamide and (2R,3R)-2-amino-3-methyl-N-(2-morpholinoethyl)-pentanamide, or a pharmaceutically acceptable salt, solvate, ester, or prodrug thereof; then (2S,3 S)-2-amino-3-methyl-N-(2-morpholinoethyl)-pentanamide, or a pharmaceutically acceptable salt, solvate, ester, or prodrug thereof, is in an amount not less than about 5% by weight. In one embodiment, the mixture consists of any two of the aforementioned four compounds. In another embodiment, the mixture consists of any three of the aforementioned four compounds. In another embodiment, the mixture consists of the aforementioned four compounds. Subject to the above-mentioned proviso, the individual compounds in the mixture can be in any ratio or weight percentage. In one embodiment, any of the two or more compounds in the mixture is in an amount of about 0.5% by weight or more. In another embodiment, any of the two or more compounds in the mixture is in an amount of about 5% by weight or more. In another embodiment, each of the two or more compounds in the mixture is in an approximately equal amount.

Scheme A provides the chemical structures of the above-mentioned compounds.

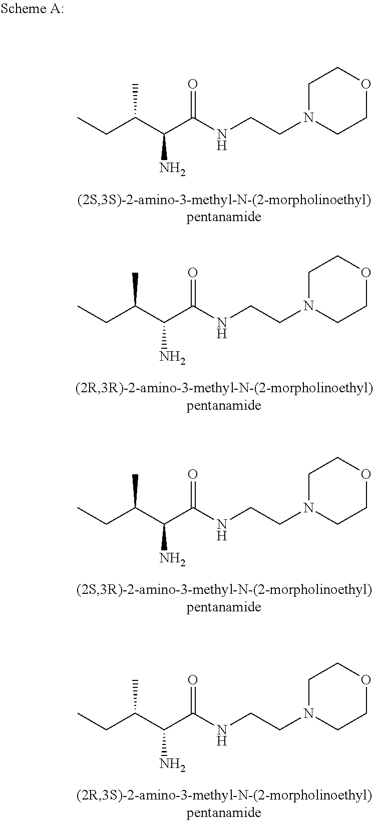 [SMILES: CC[C@H](C)[C@H](N)C(=O)NCCN1CCOCC1 ; CC[C@@H](C)[C@@H](N)C(=O)NCCN1CCOCC1 ; CC[C@@H](C)[C@H](N)C(=O)NCCN1CCOCC1 ; CC[C@H](C)[C@@H](N)C(=O)NCCN1CCOCC1]

In one embodiment, the present invention provides a mixture of (2R,3R)-2-amino-3-methyl-N-(2-morpholinoethyl)-pentanamide and (2S,3 S)-2-amino-3-methyl-N-(2-morpholinoethyl)-pentanamide, or a pharmaceutically acceptable salt, solvate, ester, or prodrug thereof, with the proviso that (2S,3 S)-2-amino-3-methyl-N-(2-morpholinoethyl)-pentanamide, or a pharmaceutically acceptable salt, solvate, ester, or prodrug thereof, is in an amount not less than about 5% by weight based on the total amount of the mixture. Subject to the above-mentioned proviso, the individual compounds in the mixture can be in any ratio or weight percentage. In one specific embodiment, the mixture consists of (2R,3R)-2-amino-3-methyl-N-(2-morpholinoethyl)-pentanamide and (2S,3 S)-2-amino-3-methyl-N-(2-morpholinoethyl)-pentanamide, or a pharmaceutically acceptable salt, solvate, ester, or prodrug thereof, in an approximately equal amount.

In one embodiment, the present invention provides a mixture of (2R,3 S)-2-amino-3-methyl-N-(2-morpholinoethyl)-pentanamide and (2S,3R)-2-amino-3-methyl-N-(2-morpholinoethyl)-pentanamide, or a pharmaceutically acceptable salt, solvate, ester, or prodrug thereof. The individual compounds in the mixture can be in any ratio or weight percentage. In one specific embodiment, the mixture consists of (2R,3 S)-2-amino-3-methyl-N-(2-morpholinoethyl)-pentanamide and (2S,3R)-2-amino-3-methyl-N-(2-morpholinoethyl)-pentanamide, or a pharmaceutically acceptable salt, solvate, ester, or prodrug thereof.

In another aspect, the present invention provides a pharmaceutical composition comprising the compound selected from the group consisting of (2R,3R)-2-amino-3-methyl-N-(2-morpholinoethyl)-pentanamide; (2R,3 S)-2-amino-3-methyl-N-(2-morpholinoethyl)-pentanamide; and (2S,3R)-2-amino-3-methyl-N-(2-morpholinoethyl)-pentanamide, or a pharmaceutically acceptable salt, solvate, ester, or prodrug thereof; and a pharmaceutically acceptable carrier.

In another aspect, the present invention provides a pharmaceutical composition comprising a mixture of two or more compounds selected from the group consisting of (2S,3 S)-2-amino-3-methyl-N-(2-morpholinoethyl)-pentanamide; (2R,3R)-2-amino-3-methyl-N-(2-morpholinoethyl)-pentanamide; (2R,3 S)-2-amino-3-methyl-N-(2-morpholinoethyl)-pentanamide; and (2S,3R)-2-amino-3-methyl-N-(2-morpholinoethyl)-pentanamide; or a pharmaceutically acceptable salt, solvate, ester, or prodrug thereof; and a pharmaceutically acceptable carrier, with the proviso that when the mixture consists of (2S,3 S)-2-amino-3-methyl-N-(2-morpholinoethyl)-pentanamide and (2R,3R)-2-amino-3-methyl-N-(2-morpholinoethyl)-pentanamide, or a pharmaceutically acceptable salt, solvate, ester, or prodrug thereof; then (2S,3 S)-2-amino-3-methyl-N-(2-morpholinoethyl)-pentanamide, or a pharmaceutically acceptable salt, solvate, ester, or prodrug thereof, is in an amount not less than about 5% by weight.

The application provides compounds having a modulating effect on p75NTR. These compounds, along with related pharmaceutical compounds and methods, are useful in the treatment and prevention of alcohol use disorder and associated diseases.

A set of compounds disclosed herein are labeled as follows:

TABLE I
Structures of Compounds i-vii

| Compound | Name |
|---|---|
| 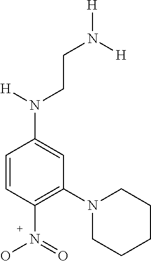 | Compound (i) (also referred to herein as “LM11A-28”) |
| 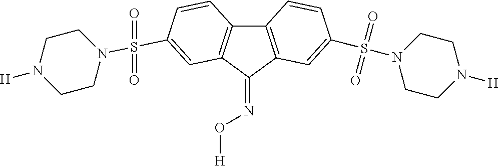 [SMILES: O=S(=O)(c1ccc2c(c1)C(=NO)c1cc(S(=O)(=O)N3CCNCC3)ccc1-2)N1CCNCC1] | Compound (ii) (also referred to herein as “LM11A-7”) |
| 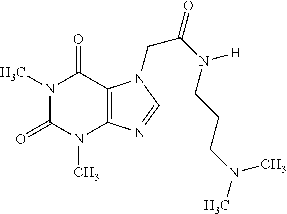 | Compound (iii) (also referred to herein as “LM11A-24”, “24”, and “C24”) |
| 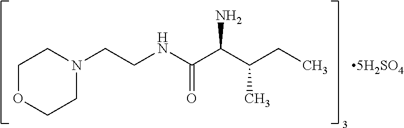 | Compound (X) (also referred to herein as “LM11A-31”) |
| 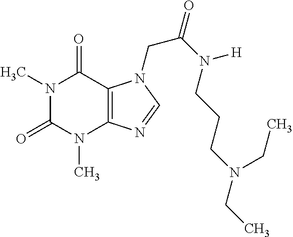 [SMILES: CCN(CC)CCCNC(=O)Cn1cnc2c1c(=O)n(C)c(=O)n2C] | Compound (v) (also referred to herein as “LM11A-36”, “36”, and “C36”) |
| 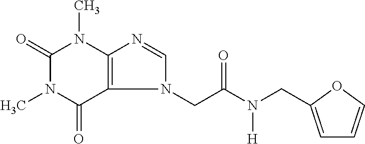 [SMILES: Cn1c(=O)c2c(ncn2CC(=O)NCc2ccco2)n(C)c1=O] | Compound (vi) (also referred to herein as “LM11A-38” and “C38”) |
| 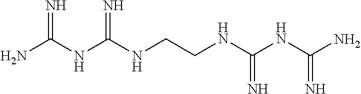 [SMILES: N=C(N)NC(=N)NCCNC(=N)NC(=N)N] | Compound (vii) |

In one variation of any of the disclosed aspects or embodiments, the compound administered to a subject in need thereof is selected from the group consisting of:

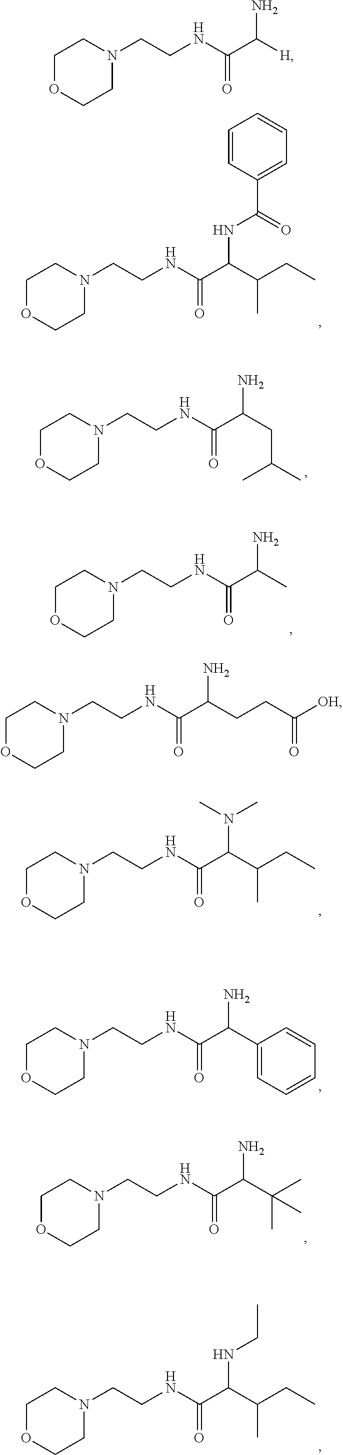

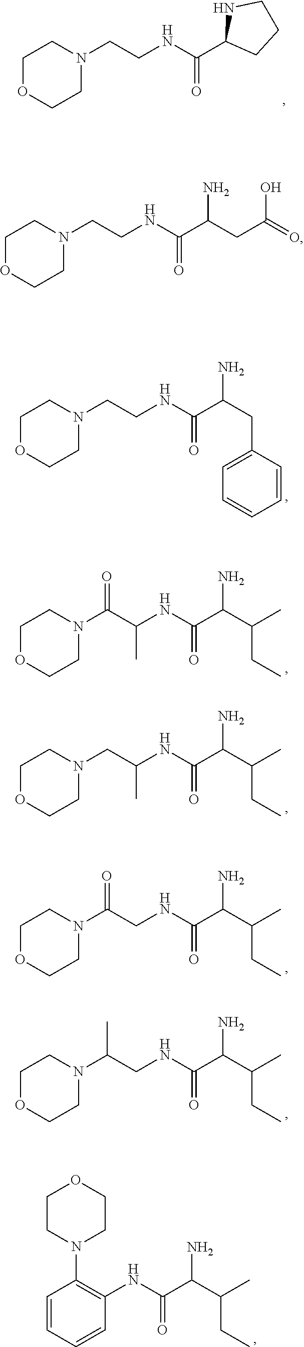

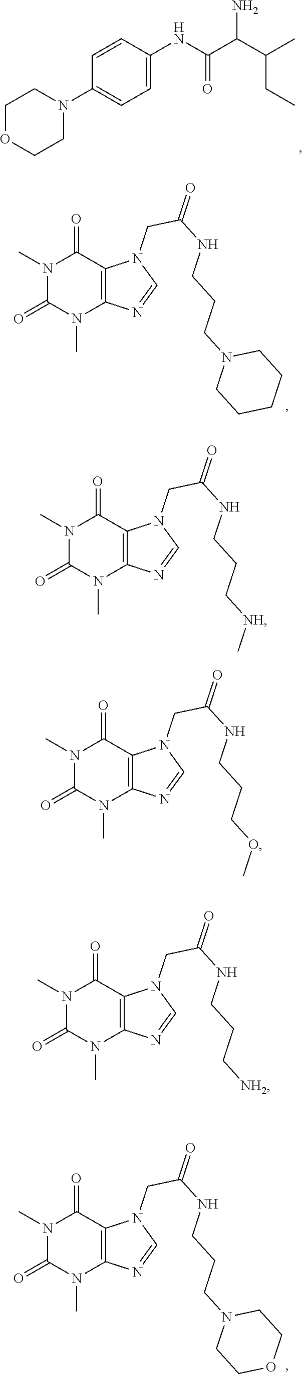

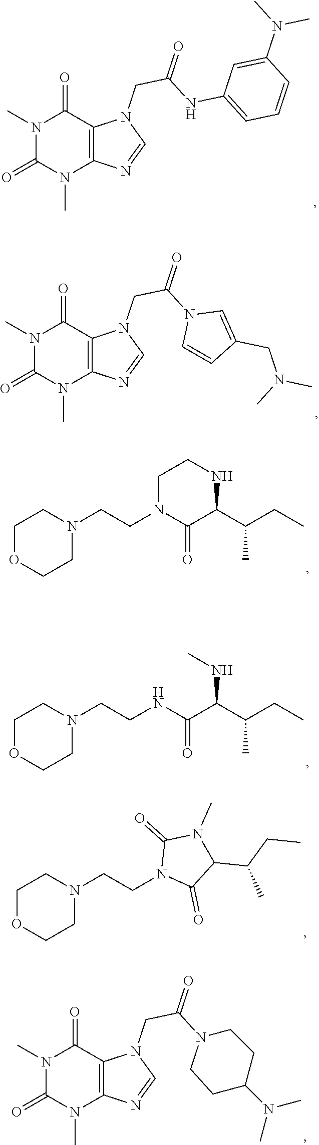

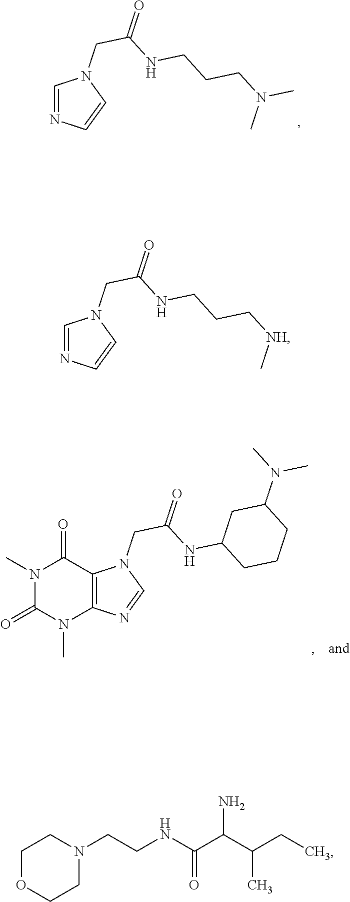

or a pharmaceutically acceptable salt of any one thereof.

In another variation of any of the disclosed embodiments or aspects, the compound administered to a subject in need thereof is selected from the group consisting of:

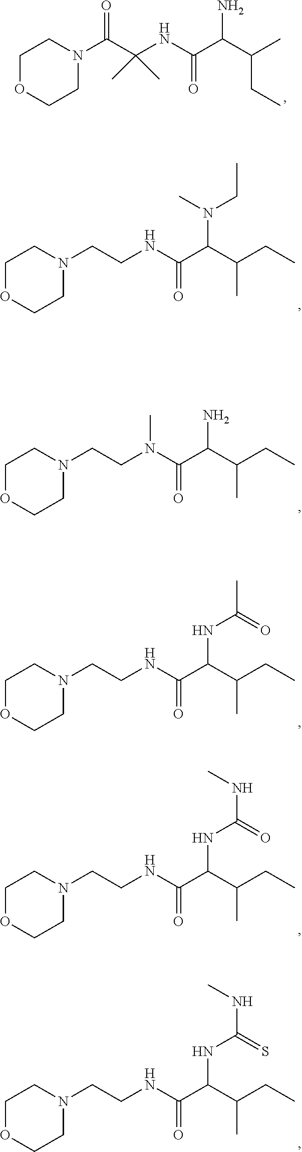 [SMILES: CCC(C)C(N)C(=O)NC(C)(C)C(=O)N1CCOCC1 ; CCC(C)C(C(=O)NCCN1CCOCC1)N(C)CC ; CCC(C)C(N)C(=O)N(C)CCN1CCOCC1 ; CCC(C)C(NC(C)=O)C(=O)NCCN1CCOCC1 ; CCC(C)C(NC(=O)NC)C(=O)NCCN1CCOCC1 ; CCC(C)C(NC(=S)NC)C(=O)NCCN1CCOCC1]

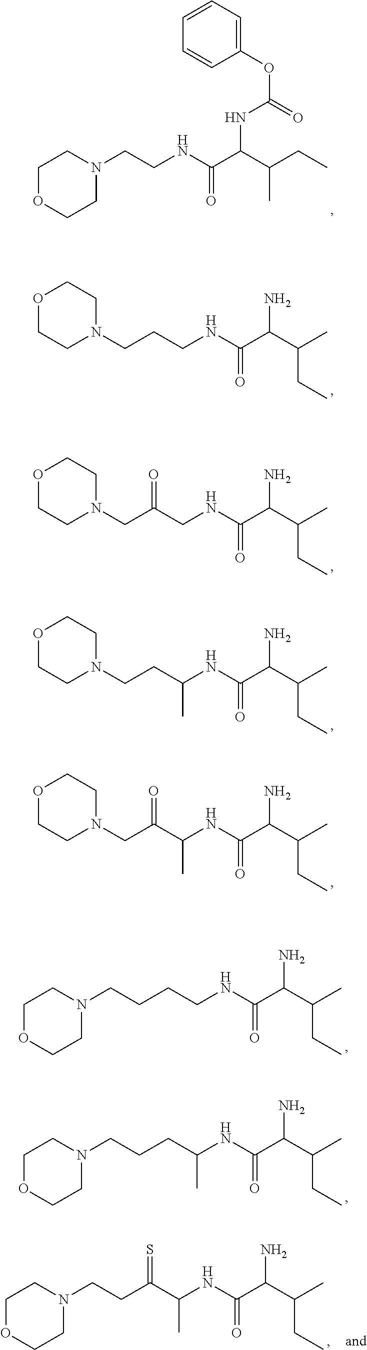

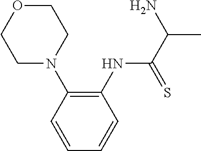 [SMILES: CC(N)C(=S)Nc1ccccc1N1CCOCC1]

or a pharmaceutically acceptable salt of any one thereof.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present invention, whether radioactive or not, are encompassed within the scope of the present invention. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically-enriched compounds may be prepared by conventional techniques well known to those skilled in the art.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

It is also to be understood that the disclosed compounds can further comprise pharmaceutically acceptable salts.

Such salts include, but are not limited to, pharmaceutically acceptable acid addition salts, pharmaceutically acceptable base addition salts, pharmaceutically acceptable metal salts, ammonium and alkylated ammonium salts.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like.

Examples of metal salts include lithium, sodium, potassium, magnesium salts and the like.

Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like.

Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, Pa. In certain embodiments, the compound of the disclosure is a sulfuric acid addition salt. In certain embodiments, the compound of the disclosure is a sulfuric acid addition salt of a compound of Formula I, IA, IB, II, IIA, IIB, III, IV, IVA, or X. The compound of the disclosure may be a sulfuric acid addition salt of 2 amino-3-methyl-N-(2-morpholinoethyl)-pentanamide, such as a sulfuric acid addition salt of (2R,3R)-2-amino-3-methyl-N-(2-morpholinoethyl)-pentanamide, a sulfuric acid addition salt of (2R,3 S)-2-amino-3-methyl-N-(2-morpholinoethyl)-pentanamide, a sulfuric acid addition salt of (2S,3R)-2-amino-3-methyl-N-(2-morpholinoethyl)-pentanamide, or a sulfuric acid addition salt of (2S,3 S)-2-amino-3-methyl-N-(2-morpholinoethyl)-pentanamide.

The compounds disclosed herein can also encompass derivatives of a parent compound, which can modulate p75NTR. The derivative can exhibit enhancement in at least one of the characteristics selected from the group consisting of hydrophilicity, lipophilicity, amphipathicity, solubility, bioavailability, and resistance to hepatic degradation, as compared to the parent compound.

The aforementioned individual compounds or mixtures can be used for treating a wide range of conditions, diseases, and disorders described herein.

In one aspect, there is provided a pharmaceutical composition comprising a pharmaceutically acceptable diluent or carrier and a compound of Formula I, IA, IB, II, IIA, IIB, III, IV, IVA, or X or a pharmaceutically acceptable salt, ester, prodrug or solvate thereof.

### Compositions of Disclosure

### Formulations

For the purposes of this invention, the compounds may be administered by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The compounds disclosed herein can be formulated in accordance with the routine procedures adapted for desired administration route. Accordingly, the compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compounds can further be formulated for topical administration. Suitable topical formulations include one or more compounds in the form of a liquid, lotion, cream or gel. Topical administration can be accomplished by application directly on the treatment area. For example, such application can be accomplished by rubbing the formulation (such as a lotion or gel) onto the skin of the treatment area, or by spray application of a liquid formulation onto the treatment area.

In some formulations, bioimplant materials can be coated with the compounds so as to improve interaction between cells and the implant.

Formulations of the compounds can contain minor amounts of wetting or emulsifying agents, or pH buffering agents. The formulations comprising the compound can be a liquid solution, suspension, emulsion, tablet, pill, capsule, sustained release formulation, or powder.

The compounds can be formulated as a suppository, with traditional binders and carriers such as triglycerides.

Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Oral formulations can include standard carriers such as pharmaceutical grades of mannitol, lactose, starch, magnesium stearate, polyvinyl pyrrolidone, sodium saccharine, cellulose, magnesium carbonate, etc. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

The pharmaceutical formulations comprising the compounds of the present application can include an agent which controls release of the compound, thereby providing a timed or sustained release compound.

### Carriers

Pharmaceutically acceptable carriers are well known to those skilled in the art and include, but are not limited to, from about 0.01 to about 0.1 M and preferably 0.05M phosphate buffer or 0.8% saline. Such pharmaceutically acceptable carriers can be aqueous or non-aqueous solutions, suspensions and emulsions.

Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

### Treatment Methods of Disclosure

### Methods of Use

As disclosed throughout, the present disclosure provides treatment of disorders associated with p75NTR-mediated signaling. The present disclosure provides treatment of alcohol use disorder and associated diseases by administering compounds that modulate p75NTR. Additionally disclosed are methods for treating alcohol use disorder and associated diseases involving compounds that modulate p75NTR in the DLS.

In some aspects, the present disclosure provides a method for treating alcohol use disorders and associated diseases comprising administering a p75NTR modulator. In addition to the p75NTR modulator described in the present disclosure, p75NTR modulators also include those known in the field, such as small molecules described in U.S. Pat. Nos. 7,723,328, 9,271,985, and U.S. Publ. No. 2015/0259278, the contents of each of which are incorporated by reference herein in their entirety. In some embodiments, the p75NTR modulator inhibits p75NTR signaling. In some embodiments, the p75NTR modulator activates p75NTR signaling.

In one aspect, there is provided a method for modulating p75NTR comprising contacting a cell containing p75NTR with one or more compounds of the present application or a pharmaceutically acceptable salt, ester, solvate or prodrug thereof.

In certain embodiments, the disclosure provides a method for the treatment of alcohol use disorder and associated diseases comprising administering to a subject in need of such treatment a compound described herein, e.g., a compound of Formula I, IA, IB, II, IIA, IIB, III, IV, IVA, or X or a pharmaceutically acceptable salt, ester, solvate or prodrug thereof. In some embodiments, the compounds and methods attenuate moderate to excessive alcohol use in a subject in need thereof. In some embodiments, the disease or disorder is a disease or disorder associated with alcohol abuse, also referred to herein as excessive alcohol use. Alcohol abuse may be characterized by the habitual use of alcoholic beverages. For example, excessive alcohol use may involve the consumption of at least three or more, at least four or more, or at least five or more alcoholic beverages for at least three or more days per week. In certain embodiments, alcohol abuse may be characterized by drinking alcoholic beverages three or more days in a week, such as four or more days a week, such as five or more days a week, such as six or more days a week or consumption of alcohol every day of the week. In certain embodiments, the alcohol abuse is characterized by one or more years of excessive alcohol use, e.g., about one year or more, about two years or more, about five years or more, about six years or more, about seven years or more, about eight years or more, about nine years or more or about 10 years or more.

Alcohol abuse may be characterized by a subject having an habitually high blood alcohol content. Blood alcohol content level of the subject may be greater than 0.05% three times a week or more, such as from about 0.05-0.40%, such as from about 0.05-0.30%, such as from about 0.10-0.30%, three times a week or more. Excessive alcohol use may be characterized by a subject that is significantly physically and/or verbally impaired while under the influence of alcohol. In certain embodiments, alcohol abuse is characterized by a subject that is not significantly physically and/or verbally impaired while under the influence of alcohol. In certain embodiments, alcohol abuse may be characterized by the consumption of alcohol when the subject is under stress, depressed, or exposed to a trigger event, e.g., attending an event with alcohol, or any combination thereof.

A p75 NTR modulator, e.g., a compound of Formula I, IA, IB, II, IIA, IIB, III, IV, IVA, or X, or a pharmaceutically acceptable salt, ester, solvate or prodrug thereof as disclosed herein, may be used to treat or prevent alcohol use disorder and associated diseases. In one embodiment, the disease or disorder is a disease or disorder associated with alcohol abuse. In another embodiment, the disease or disorder is selected from alcohol abuse, alcohol dependence, alcoholism, substance use disorder, substance abuse, and substance dependence. In another embodiment, the disease or disorder is a secondary disease or disorder selected from the group consisting of alcohol use disorder and associated diseases, including, but not limited to hepatic steatosis, alcoholic hepatitis, cirrhosis, gastritis, stomach ulcers, esophageal ulcers, interference with absorption of B vitamins and other nutrients, pancreatitis, high blood pressure, enlarged heart, heart failure, stroke, atrial fibrillation, cardiovascular disease, hypoglycemia, diabetes, erectile dysfunction, interruption of menstruation, nystagmus, weakness of eye muscles, paralysis of eye muscles, thiamine deficiency, dementia, miscarriage, fetal alcohol syndrome, osteoporosis, damaged bone marrow, low platelet count, numbness and pain in body, disordered thinking, short-term memory loss, weakened immune system, infectious disease, cancer, anemia, depression, seizures, gout, nerve damages. In one embodiment, compounds of the present application are used in the prevention of alcohol use disorder (AUD).

An alcohol use disorder and associated diseases may be identified or diagnosed in a subject exhibiting a symptom related to excessive alcohol use, including: drinking more or longer than intended; inability to cut down or stop drinking after wanting or trying to; spending a considerable time on drinking, on being sick, or on overcoming other aftereffects; desire to drink impaired ability to think of anything else; drinking or being sick from drinking interfered with responsibilities including home, family, job, and school; continuing to drink despite it causing trouble with family or friends; giving up or cutting back on important, interesting, or pleasurable activities in order to drink; entered into situations while under the influence or after drinking that increased opportunity of being harmed; continuing to drink despite it causing health issues, including depression, anxiety, other health problems, and memory black out; drinking more than previously to experience the same effect, or finding that the usual number of drinks had less effect than before; experiencing withdrawal symptoms, including trouble sleeping, shakiness, restlessness, nausea, sweating, a racing heart, and a seizure, or sensed things that were not there.

In some embodiments, the disclosure provides methods for treating mild AUD, wherein mild AUD is characterized by the presence of at least two of the above mentioned symptoms. In some embodiments, the disclosure provides methods for treating moderate AUD, wherein moderate AUD is characterized by the presence of at least four of the above mentioned symptoms. In some embodiments, the disclosure provides methods for treating severe AUD, wherein severe AUD is characterized by the presence of at least six of the above mentioned symptoms.

In some embodiments, the subject has a predisposition to alcoholism. For example, the subject may be more susceptible to excessive alcohol use due to genetic factors, such as having at least one family member that has had an alcohol use disorder. A subject predisposed to excessive alcohol use may be more sensitive to stress than others, may have experienced or witnessed a traumatic event, prone to peer pressure, or may have easy access to alcohol. In certain embodiments, the methods of the disclosure may be used to attenuate or prevent excessive alcohol use in a subject predisposed to alcohol abuse, e.g., a subject with one or more relatives suffering from substance abuse or a subject recovering or recovered from an alcohol use disorder.

In certain embodiments, administering a compound or salt described herein to the subject may attenuate alcohol intake of said subject or prevent excessive alcohol consumption of said subject as determined by blood alcohol content, e.g., wherein the subject's alcohol intake does not exceed about 0.05%, does not exceed 0.06%, does not exceed 0.07%, does not exceed 0.08%, does not exceed about 0.09% or does not exceed about 0.1%, following administration of a compound or salt described herein.

In certain embodiments, administering a compound or salt described herein to the subject attenuates alcohol intake of said subject as compared with the frequency of alcohol intake prior to administering said compound or salt. In certain embodiments, administering a compound or salt of the disclosure to the subject attenuates alcohol intake of said subject as compared with the amount of alcohol intake prior to administering said compound or salt thereof. Administering said compound or salt may attenuate alcohol intake of said subject by about 10% or more, about 20% or more, about 30% or more, about 40% or more, or about 50% or more as compared with the amount of alcohol intake prior to administering said compound or salt thereof.

In certain embodiments, a compound or salt of the disclosure is administered to the subject as part of a prescribed treatment regimen, e.g., administered once a week, twice a week, three time a week, four times a week, daily, twice daily, or three times daily. The treatment regimen may be prescribed to the subject for a period of days, weeks, months or years.

In certain embodiments, the disclosure provides a method of preventing excessive alcohol consumption comprising administering a compound or salt described herein to a subject prior to, during or after a trigger event, or any combination thereof. A trigger event may be any form of stimuli that initiates the desire to engage in alcohol consumption including, but not limited to, a stressful situation, learning of bad news, the end of a workday, attending an event with alcohol, being encouraged by others to consume alcohol, etc. In certain embodiments, the administration of the compound or salt thereof for a trigger event may be in addition to the prescribed treatment regimen, e.g., a dose is administered before, during or after a trigger event to supplement a twice weekly dose. In certain embodiments, the dose administered with the trigger event may be greater than a dose of the treatment regimen.

### Methods of Administration

The present application discloses a method of administering compounds that modulating p75NTR in order to treat alcohol use disorder and associated diseases. The method can comprise the step of administering to a subject an effective amount of a compound having a modulating effect on p75NTR, such as any of the compounds disclosed herein.

As used herein, administering can be effected or performed using any of the various methods known to those skilled in the art. The compound can be administered, for example, subcutaneously, intravenously, parenterally, intraperitoneally, intradermally, intramuscularly, topically, enteral (e.g., orally), rectally, nasally, buccally, sublingually, vaginally, by inhalation spray, by drug pump or via an implanted reservoir in dosage formulations containing conventional non-toxic, physiologically acceptable carriers or vehicles.

Further, the presently disclosed compounds can be administered to a localized area in need of treatment. This can be achieved by, for example, and not by way of limitation, local infusion during surgery, topical application, transdermal patches, by injection, by catheter, by suppository, or by implant (the implant can optionally be of a porous, non-porous, or gelatinous material), including membranes, such as sialastic membranes or fibers.

The form in which the compound is administered (e.g., syrup, elixir, capsule, tablet, solution, foams, emulsion, gel, sol) will depend in part on the route by which it is administered. For example, for mucosal (e.g., oral mucosa, rectal, intestinal mucosa, bronchial mucosa) administration, nose drops, aerosols, inhalants, nebulizers, eye drops or suppositories can be used. The compound can also be used to coat bioimplantable materials to enhance neurite outgrowth, neural survival, or cellular interaction with the implant surface. The compounds and agents disclosed herein can be administered together with other biologically active agents, such as analgesics, anti-inflammatory agents, anesthetics and other agents which can control one or more symptoms or causes of a p75NTR-mediated condition.

Additionally, administration can comprise administering to the subject a plurality of dosages over a suitable period of time. Such administration regimens can be determined according to routine methods, upon a review of the instant disclosure.

The compounds of the present application can be employed as the sole active agent in a pharmaceutical or can be used in combination (e.g., administered proximate in time to each other or even in the same formulation) with other active ingredients, e.g., neurotrophins, or other factors or drugs which can modulate p75NTR.

### Dosage

Compounds of the invention may be administered in a dose of about 0.01 mg/kg/dose to about 150 mg/kg/dose. Alternately the dose can be from about 0.1 mg/kg/dose to about 10 mg/kg/dose; or about 1 mg/kg/dose to 10 mg/kg/dose. In some dosages, the compounds disclosed herein are administered at about 5 mg/kg/dose. Time release preparations may be employed or the dose may be administered in as many divided doses as is convenient. When other methods are used (e.g. intravenous administration), compounds are administered to the affected tissue at a rate from about 0.05 to about 10 mg/kg/hour, alternately from about 0.1 to about 1 mg/kg/hour. Such rates are easily maintained when these compounds are intravenously administered as discussed herein. Generally, topically administered formulations are administered in a dose of about 0.5 mg/kg/dose to about 10 mg/kg/dose range. Alternately, topical formulations are administered at a dose of about 1 mg/kg/dose to about 7.5 mg/kg/dose or even about 1 mg/kg/dose to about 5 mg/kg/dose.

A range of from about 0.1 to about 150 mg/kg is appropriate for a single dose. Continuous administration is appropriate in the range of about 0.05 to about 10 mg/kg. Topical administration is appropriate for conditions such as hair loss or wound revascularization.

Drug doses can also be given in milligrams per square meter of body surface area rather than body weight, as this method achieves a good correlation to certain metabolic and excretionary functions. Moreover, body surface area can be used as a common denominator for drug dosage in adults and children as well as in different animal species (Freireich et al., (1966) Cancer Chemother Rep. 50, 219-244). Briefly, to express a mg/kg dose in any given species as the equivalent mg/sq m dose, the dosage is multiplied by the appropriate km factor. In an adult human, 100 mg/kg is equivalent to 100 mg/kg×37 kg/sq m=3700 mg/m².

Insofar as the compounds disclosed herein can take the form of a mimetic or fragment thereof, it is to be appreciated that the potency, and therefore dosage of an effective amount can vary. However, one skilled in the art can readily assess the potency of a compound of the type presently envisioned by the present application.

In settings of a gradually progressive nervous system disorder, compounds of the present application are generally administered on an ongoing basis. In certain settings administration of a compound disclosed herein can commence prior to the development of disease symptoms as part of a strategy to delay or prevent the disease. In other settings a compound disclosed herein is administered after the onset of disease symptoms as part of a strategy to slow or reverse the disease process and/or part of a strategy to improve cellular function and reduce symptoms. Compounds have been developed that cross the blood brain barrier and hence would be delivered by oral administration or by other peripheral routes. Compounds that do not cross the blood brain barrier are applied for targets outside of the central nervous system. For targets and tissues outside of the nervous system, compounds are applied in either acute or chronic settings by other oral or directed target administration such as by topical application.

It will be appreciated by one of skill in the art that dosage range will depend on the particular compound, and its potency. The dosage range is understood to be large enough to produce the desired effect in which the neurodegenerative or other disorder and the symptoms associated therewith are ameliorated and/or survival of the cells is achieved, but not be so large as to cause unmanageable adverse side effects. It will be understood, however, that the specific dose level for any particular subject will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those skilled in the art. The dosage can also be adjusted by the individual physician in the event of any complication. No unacceptable toxicological effects are expected when compounds disclosed herein are used in accordance with the present application.

An effective amount of the compounds disclosed herein comprise amounts sufficient to produce a measurable biological response. Actual dosage levels of active ingredients in a therapeutic compound of the present application can be varied so as to administer an amount of the active compound that is effective to achieve the desired therapeutic response for a particular subject and/or application. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

Further with respect to the methods of the present application, a preferred subject is a vertebrate subject. A preferred vertebrate is warm-blooded; a preferred warm-blooded vertebrate is a mammal. The subject treated by the presently disclosed methods is desirably a human, although it is to be understood that the principles of the present application indicate effectiveness with respect to all vertebrate species which are to include in the term “subject.” In this context, a vertebrate is understood to be any vertebrate species in which treatment of a neurodegenerative disorder is desirable. As used herein, the term “subject” includes both human and animal subjects. Thus, veterinary therapeutic uses are provided in accordance with the present application.

As such, the present application provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

### Combination Therapies

In some aspects, the present disclosure provides the compounds and salts of any one of Formulas I, IA, IB, II, IIA, IIB, III, IV, IVA, or X and compositions thereof in combination with behavioral therapy. In one embodiment, the subject has been admitted into an alcohol treatment center. The subject may be receiving inpatient or outpatient services. In another embodiment, the subject has enrolled into a rehab program, such as group therapy and the 12 step program. The behavioral therapy may provide support, education and/or a plan for discontinuing alcohol use.

The compounds and salts of any one of Formulas I, IA, IB, II, IIA, IIB, III, IV, IVA, or X and compositions thereof, may also be used in combination with other therapeutic agents that are selected for their therapeutic value for the condition to be treated. In some embodiments, a compound or salt of the present disclosure is combined with a p75NTR modulator. For example, a compound or salt of the present disclosure may be combined with one or more additional therapeutic agents selected from disulfiram (Antabuse®), oral naltrexone, extended-release naltrexone (Vivitrol®), and acamprosate (Campral®).

Combination therapies may be administered concurrently as a single composition. Combination therapies may be administered together, at or near the same time, but in separate pharmaceutical compositions. The determination of the mode of administration and the advisability of administration, where possible, in the same pharmaceutical composition, is well within the knowledge of the clinician. The initial administration can be made according to established protocols recognized in the field, and then, based upon the observed effects, the dosage, modes of administration and times of administration can be modified by the clinician.

In certain instances, it may be appropriate to administer at least one pharmaceutical agent described herein in combination with another therapeutic agent. By way of example only, if one of the side effects experienced by a subject upon receiving one of the pharmaceutical agents herein, such as a compound or salt of any one of Formulas (I, IA, IB, II, IIA, IIB, III, IV, IVA or X is nausea, then it may be appropriate to administer an anti-nausea agent in combination with the initial therapeutic agent. Or, by way of example only, the therapeutic effectiveness of one of the pharmaceutical agents described herein may be enhanced by administration of an adjuvant, i.e., by itself the adjuvant may have minimal therapeutic benefit, but in combination with another therapeutic agent, the overall therapeutic benefit to the subject is enhanced. Or, by way of example only, the benefit experienced by a subject may be increased by administering one of the pharmaceutical agents described herein with another therapeutic agent, which also includes a therapeutic regimen, that also has therapeutic benefit. In any case, regardless of the disease, disorder or condition being treated, the overall benefit experienced by the subject may simply be additive of the two agents or the subject may experience a synergistic benefit.

The particular choice of pharmaceutical agents used will depend upon the diagnosis of the attending physicians and their judgment of the condition of the subject and the appropriate treatment protocol. The pharmaceutical agents may be administered concurrently (e.g., simultaneously, essentially simultaneously or within the same treatment protocol) or sequentially, depending upon the nature of the disease, disorder, or condition, the condition of the subject, and the actual choice of pharmaceutical agents used. The determination of the order of administration, and the number of repetitions of administration of each therapeutic agent during a treatment protocol, is well within the knowledge of the physician after evaluation of the disease being treated and the condition of the subject.

Therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the subject.

For combination therapies described herein, dosages of the co-administered pharmaceutical agents will of course vary depending on the type of co-drug employed, on the specific drug employed, on the disease or condition being treated and so forth. In addition, when co-administered with one or more biologically active agents, the pharmaceutical agent provided herein may be administered either simultaneously with the pharmaceutical agent, or sequentially. If administered sequentially, the attending physician will decide on the appropriate sequence of administering the pharmaceutical agent in combination with a biologically active agent(s).

In any case, the multiple therapeutic agents (one of which is a compound or salt of any one of Formulas I, IA, IB, II, IIA, IIB, III, IV, IVA or X described herein) may be administered in any order or even simultaneously. If simultaneously, the multiple therapeutic agents may be provided in a single, unified form, or in multiple forms, by way of example only, either as a single pill or as two separate pills. One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents; the use of multiple therapeutic combinations are also envisioned.

It is understood that the dosage regimen to treat, prevent, or ameliorate the condition(s) for which relief is sought, can be modified in accordance with a variety of factors. These factors include the disorder or condition from which the subject suffers, as well as the age, weight, sex, diet, and medical condition of the subject. Thus, the dosage regimen actually employed can vary widely and therefore can deviate from the dosage regimens set forth herein.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either pharmaceutical agent being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval.

In addition, the pharmaceutical agents described herein also may be used in combination with procedures that may provide additional or synergistic benefit to the subject. By way of example only, subjects are expected to find therapeutic and/or prophylactic benefit in the methods described herein, wherein pharmaceutical composition of a pharmaceutical agent disclosed herein and/or combinations with other therapeutics are combined with genetic testing to determine whether that individual is a carrier of a mutant gene that is known to be correlated with certain diseases or conditions.

The pharmaceutical agents described herein and combination therapies can be administered before, during or after the occurrence of a disease or condition, and the timing of administering the composition containing a pharmaceutical agent can vary. Thus, for example, the pharmaceutical agent can be used as a prophylactic and can be administered continuously to subjects with a propensity to develop conditions or diseases in order to prevent the occurrence of the disease or condition. The pharmaceutical agents and compositions can be administered to a subject during or as soon as possible after the onset of the symptoms. The administration of the pharmaceutical agents can be initiated within the first 48 hours of the onset of the symptoms, such as within the first 48 hours of the onset of the symptoms, such as within the first 6 hours of the onset of the symptoms, such as within 3 hours of the onset of the symptoms. The initial administration can be via any route practical, such as, for example, an intravenous injection, a bolus injection, infusion over about 5 minutes to about 5 hours, a pill, a capsule, transdermal patch, buccal delivery, and the like, or combination thereof. A pharmaceutical agent may be administered as soon as is practicable after the onset of a disease or condition is detected or suspected, and for a length of time necessary for the treatment of the disease, such as, for example, from 1 day to about 3 months. The length of treatment can vary for each subject, and the length can be determined using the known criteria. For example, the pharmaceutical agent or a formulation containing the pharmaceutical agent can be administered for at least 2 weeks, such as about 1 month to about 5 years.

A compound or salt of any one of Formulas I, IA, IB, II, IIA, IIB, III, IV, IVA or X may be used together with a small molecule compound that modulates p75NTR mediated signaling pathway. As described herein, methods are also provided for treating excessive alcohol use diseases or disorders, wherein the methods comprise administering to a subject in need thereof at least one compound or salt of any one of Formulas I, IA, IB, II, IIA, IIB, III, IV, IVA or X.

### Examples

### General Syntheses

Standard procedures and chemical transformation and related methods are well known to one skilled in the art, and such methods and procedures have been described, for example, in standard references such as Fiesers' Reagents for Organic Synthesis, John Wiley and Sons, New York, N.Y., 2002; Organic Reactions, vols. 1-83, John Wiley and Sons, New York, N.Y., 2006; March J. and Smith M., Advanced Organic Chemistry, 6th ed., John Wiley and Sons, New York, N.Y.; and Larock R. C., Comprehensive Organic Transformations, Wiley-VCH Publishers, New York, 1999. All texts and references cited herein are incorporated by reference in their entirety.

Reactions using compounds having functional groups may be performed on compounds with functional groups that may be protected. A “protected” compound or derivatives means derivatives of a compound where one or more reactive site or sites or functional groups are blocked with protecting groups. Protected derivatives are useful in the preparation of the compounds of the present invention or in themselves; the protected derivatives may be the biologically active agent. An example of a comprehensive text listing suitable protecting groups may be found in T. W. Greene, Protecting Groups in Organic Synthesis, 3rd edition, John Wiley & Sons, Inc. 1999.

Preparation of many of the compounds, e.g. Compounds 1-21, can be illustrated in the general Scheme 1 below:

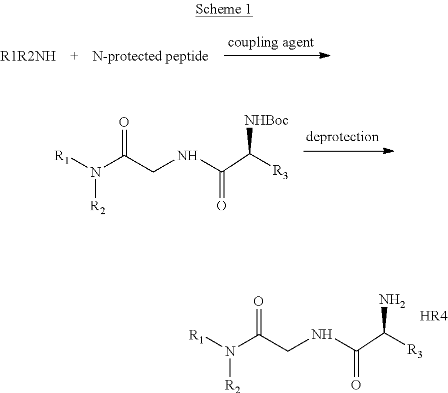

Generally the protection group for the amino acid is a Boc group. The coupling agent can be HATU, HBTU, EDC/HOBt, or DCC/DMAP. The deprotection reagent can be 4 M HCl in MeOH, 4M HCl in water, or TFA in DCM.

Generally, an amine or aniline is coupled with an N-protected amino acid and this coupled intermediate is deprotected to give a final compound or another intermediate. The second intermediate can be further modified or directly go through this coupling-deprotection cycle one more time to give the final compound.

### Example 1: Preparation of Compound 1

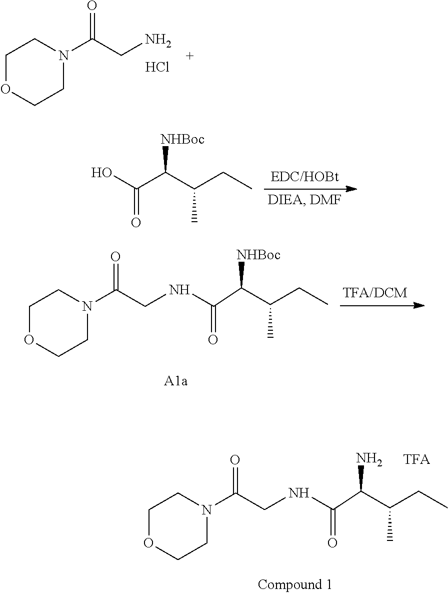

### Preparation of Intermediate A1a

| | Chemicals/Reagents | | | | | |
|---|---|---|---|---|---|---|
| S. No. | & Solvents | MW | mmol | Eq. | Amts | |
| 1 | Boc-Ile-OH | 231.29 | 1 | 1.0 | 231 | mg |
| 2 | DCM (anhydrous) | | | | 5 | mL |
| 3 | DIEA, d = 0.742 | 129.25 | 2.5 | 2.5 | 0.44 | mL |
| 4 | EDC | 191.7 | 1 | 1.0 | 192 | mg |
| 5 | HOBt | 135.13 | 1 | 1.0 | 135 | mg |
| 6 | Amine | 180.63 | 1 | 1.0 | 180 | mg |

To a solution of Boc-Ile-OH in DCM were added DIEA, HOBt, EDC, and amine in that order. The reaction mixture was stirred for 3 hours at room temperature (RT).

After the reaction was complete (LC-MS), 10% citric acid was added to quench the reaction. The DCM layer was separated and washed with saturated NaHCO₃, brine, dried with anhydrous Na₂SO₄, filtered, and concentrated. The intermediate Ala was obtained as an off-white solid (303 mg).

### Preparation of Compound 1

| | | | | | |
|---|---|---|---|---|---|
| | Chemicals/Reagents | | | | |
| S. No. | & Solvents | MW | mmol | Eq. | Amts |

| | | | |
|---|---|---|---|
| 1 | A1a | 300 | mg |
| 2 | TFA/DCM (1:1) | 4 | mL |

Cold TFA/DCM was added to the residue of Ala. The reaction mixture was stirred for 2 hours at room temperature. After the reaction was complete (LC-MS), the mixture was concentrated to afford the final compound (272 mg). Compound 1 was characterized by ¹H NMR, LC-MS and HPLC. ¹H NMR (MeOD), δ: 4.07-4.21 (m, 2H), 3.77 (d, J=5.84 Hz, 1H), 3.65-3.70 (m, 4H), 3.56-3.58 (m, 2H), 3.45-3.51 (m, 2H), 1.88-1.98 (m, 1H), 1.56-1.66 (m, 1H), 1.21-1.31 (m, 1H), 1.05 (d, J=6.78 Hz, 3H), 0.99 (t, J=7.40 Hz, 3H). LC-MS, (M+1), 258. HPLC (>95%, retention time, 1.99 min).

### Example 2: Preparation of Compound 2

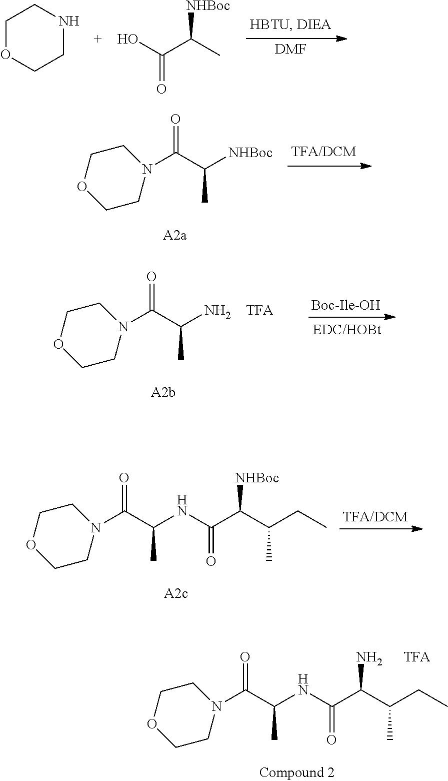

### Preparation of Intermediate A2a

| | Chemicals/Reagents | | | | | |
|---|---|---|---|---|---|---|
| S. No. | & Solvents | MW | mmol | Eq. | Amts | |
| 1 | Boc-Ala-OH | 189.21 | 2 | 1.0 | 378 | mg |
| 2 | DMF (anhydrous) | | | | 5 | mL |
| 3 | DIEA, d = 0.742 | 129.25 | 5 | 2.5 | 0.87 | mL |
| 4 | HBTU | 379 | 2 | 1.0 | 758 | mg |
| 5 | Morpholine, d = 0.99 | 87.12 | 2 | 1.0 | 175 | uL |

To a solution of Boc-Ala-OH in DMF were added HBTU, DIEA, and morpholine in that order. The reaction mixture was stirred for 2 hours at room temperature. After the reaction was complete (LC-MS), the reaction mixture was subject to prep-HPLC purification to afford the intermediate A2a (333 mg).

### Preparation of Intermediate A2b

| | | | | | |
|---|---|---|---|---|---|
| | Chemicals/Reagents | | | | |
| S. No. | & Solvents | MW | mmol | Eq. | Amts |

| | | | |
|---|---|---|---|
| 1 | A2a | 158 | mg |
| 2 | TFA/DCM (1:1) | 4 | mL |

Cold TFA/DCM was added to the residue of A2a. The reaction mixture was stirred for 2 hours at room temperature. After the reaction was complete (LC-MS), the mixture was concentrated to afford the intermediate A2b (180 mg).

### Preparation of Intermediate A2c

| | Chemicals/Reagents | | | | | |
|---|---|---|---|---|---|---|
| S. No. | & Solvents | MW | mmol | Eq. | Amts | |
| 1 | Boc-Ile-OH | 231.29 | 0.611 | 1.0 | 141 | mg |
| 2 | DCM (anhydrous) | | | | 5 | mL |
| 3 | DIEA, d = 0.742 | 129.25 | 1.83 | 3.0 | 0.32 | mL |
| 4 | HOBt•H₂O | 135.13 | 0.611 | 1.0 | 83 | mg |
| 5 | EDC | 191.7 | 0.611 | 1.0 | 117 | mg |
| 6 | A2b | | 0.611 | 1.0 | 180 | mg |

To the residue of A2b were added DCM, Boc-Ala-OH, HOBt, EDC, and DIEA in that order. The reaction mixture was stirred for 2 hours at room temperature. After the reaction was complete (LC-MS), 0.5 HCl was added to quench the reaction. The DCM layer was separated and washed with saturated NaHCO₃, brine, dried with anhydrous Na₂SO₄ and filtered. The liquid was concentrated to afford the intermediate A2c (64 mg).

### Preparation of Compound 2

| | | | | | |
|---|---|---|---|---|---|
| | Chemicals/Reagents | | | | |
| S. No. | & Solvents | MW | mmol | Eq. | Amts |

| | | | |
|---|---|---|---|
| 1 | A2c | 60 | mg |
| 2 | TFA/DCM (1:1) | 4 | mL |

Cold TFA/DCM was added to the residue of A2c. The reaction mixture was stirred for 3 hours at room temperature. After the reaction was complete (LC-MS), the mixture was concentrated to afford the final compound (51 mg). Compound 2 was characterized by ¹H NMR, LC-MS and HPLC. ¹H NMR (D₂O), δ: 3.62-3.92 (m, 10H), 1.99-2.05 (m, 1H), 1.54-1.60 (m, 1H), 1.41 (d, J=7.12 Hz, 2H), 1.26-1.33 (m, 1H), 1.06 (d, J=6.92 Hz, 3H), 0.99 (t, J=7.38 Hz, 3H). LC-MS, (M+1), 272. HPLC (>95%, retention time, 1.94 min).

### Example 3: Preparation of Compound 3

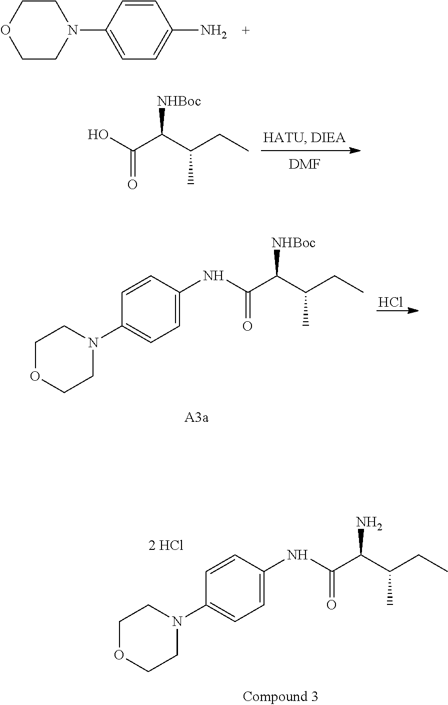

### Preparation of Intermediate A3a

| | Chemicals/Reagents | | | | | |
|---|---|---|---|---|---|---|
| S. No. | & Solvents | MW | mmol | Eq. | Amts | |
| 1 | Boc-Ile-OH | 231.29 | 0.432 | 1.0 | 100 | mg |
| 2 | DMF (anhydrous) | | | | 3 | mL |
| 3 | DIEA, d = 0.742 | 129.25 | 1.08 | 2.5 | 0.20 | mL |
| 4 | HATU | 380.2 | 0.43 | 1.0 | 163 | mg |
| 5 | 4-morpholinoaniline | 178.24 | 0.432 | 1.0 | 77 | mg |

To a solution of Boc-Ile-OH in DMF were added HATU, 4-morpholinoaniline, and DIEA in that order. The reaction mixture was stirred for 1 hour at room temperature.

After the reaction was complete (LC-MS), saturated aqueous NaHCO₃ was added to quench the reaction and EtOAc was used to extract the product. The organic layer was separated and washed with water, brine, dried with anhydrous Na₂SO₄, filtered, and concentrated. The intermediate A3a was obtained as an off-white solid (166 mg).

### Preparation of Compound 3

| | | | | | |
|---|---|---|---|---|---|
| | Chemicals/Reagents | | | | |
| S. No. | & Solvents | MW | mmol | Eq. | Amts |

| | | | |
|---|---|---|---|
| 1 | A3a | 160 | mg |
| 2 | HCl in MeOH | 4 | mL |

Cold HCl solution in MeOH was added to the residue of A3a. The reaction mixture was stirred for 3 hours at room temperature.

After the reaction was complete (LC-MS), the mixture was concentrated to afford the final compound 3 (120 mg). Compound 3 was characterized by ¹H NMR, LC-MS and HPLC. ¹H NMR (MeOD), δ: 7.88 (d, J=9.16 Hz, 2H), 7.69 (d, J=9.12 Hz, 2H), 4.09-4.11 (m, 4H), 3.92 (d, J=5.80 Hz, 1H), 3.66-3.69 (m, 4H), 2.03-2.10 (m, 1H), 1.62-1.69 (m, 1H), 1.24-1.32 (m, 1H), 1.11 (d, J=6.92 Hz, 3H), 1.01 (t, J=7.40 Hz, 3H). LC-MS, (M+1), 292. HPLC (>95%, retention time, 4.86 min).

### Example 4: Preparation of Compound 4

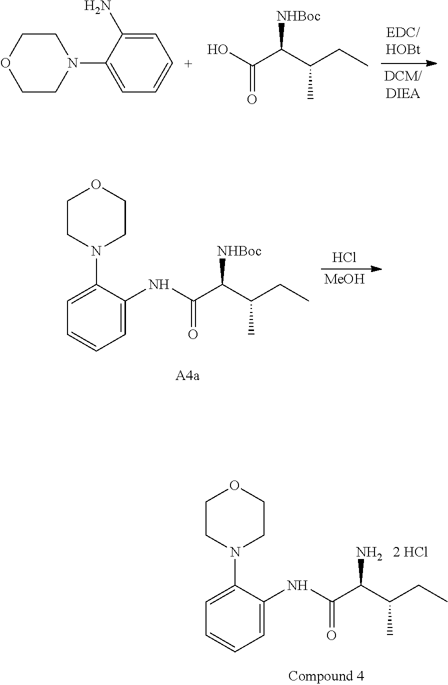 [SMILES: "MeOH" ; NC1=C[C+]=CC=C1N1CCOCC1 ; CC[C@H](C)[C@H](NC(=O)OC(C)(C)C)C(=O)O ; CC[C@H](C)[C@H](NC(=O)OC(C)(C)C)C(=O)Nc1ccccc1N1CCOCC1 ; Cl ; CC[C@H](C)[C@H](N)C(=O)Nc1ccccc1N1CCOCC1]

### Preparation of Intermediate A4a

| | Chemicals/Reagents | | | | | |
|---|---|---|---|---|---|---|
| S. No. | & Solvents | MW | mmol | Eq. | Amts | |
| 1 | Boc-Ile-OH | 231.29 | 1 | 1.0 | 231 | mg |
| 2 | DCM (anhydrous) | | | | 5 | mL |
| 3 | DIEA, d = 0.742 | 129.25 | 2.5 | 2.5 | 0.44 | mL |
| 4 | HOBt•H₂O | 135.13 | 1 | 1.0 | 135 | mg |
| 5 | EDC | 191.7 | 1 | 1.0 | 192 | mg |
| 6 | 2-morpholinoaniline | 178.23 | 0.96 | 0.96 | 170 | mg |

To the solution of Boc-Ile-OH in DCM were added HOBt, EDC, DIEA, and 2-morpholinoaniline in that order. The reaction mixture was stirred for 7.5 hours at room temperature.

The reaction mixture was subject to prep-HPLC purification to afford the intermediate A4a (94 mg).

### Preparation of Compound 4

| | | | | | |
|---|---|---|---|---|---|
| | Chemicals/Reagents | | | | |
| S. No. | & Solvents | MW | mmol | Eq. | Amts |

| | | | |
|---|---|---|---|
| 1 | A4a | 60 | mg |
| 2 | HCl in MeOH | 4 | mL |

Cold HCl solution in MeOH was added to the residue of A4a. The reaction mixture was stirred for 3 hours at room temperature.

After the reaction was complete (LC-MS), the mixture was concentrated to afford the final compound 4 (73 mg). Compound 4 was characterized by ¹H NMR, LC-MS and HPLC. ¹H NMR (D₂O), δ: 7.67 (dd, J=7.96 and 1.48 Hz, 1H), 7.49 (dd, J=8.10 and 1.34 Hz, 1H), 7.42 (dt, J=7.75 and 1.53 Hz, 1H), 7.34 (dt, J=7.67 and 1.43 Hz, 1H), 4.22 (d, J=5.00 Hz, 1H), 3.95-3.97 (m, 4H), 3.13-3.15 (m, 4H), 2.11-2.17 (m, 1H), 1.57-1.64 (m, 1H), 1.30-1.39 (m, 1H), 1.12 (d, J=6.96 Hz, 3H), 1.00 (t, J=7.40 Hz, 3H). LC-MS, (M+1), 292. HPLC (>95%, retention time, 5.31 min).

### Example 5: Preparation of Compound 5

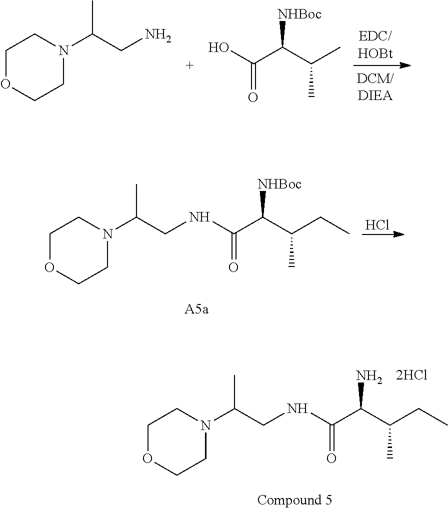

### Preparation of Intermediate A5a

| | Chemicals/Reagents | | | | | |
|---|---|---|---|---|---|---|
| S. No. | & Solvents | MW | mmol | Eq. | Amts | |
| 1 | Boc-Ile-OH | 231.29 | 1 | 1.0 | 231 | mg |
| 2 | DCM (anhydrous) | | | | 5 | mL |
| 3 | DIEA, d = 0.742 | 129.25 | 2.5 | 2.5 | 0.44 | mL |
| 4 | HOBt•H₂O | 135.13 | 1 | 1.0 | 135 | mg |
| 5 | EDC | 191.7 | 1 | 1.0 | 192 | mg |
| 6 | 2-morpholin-4-yl-propylamine | 144.22 | | | 0.19 | mL |

To the solution of Boc-Ile-OH in DCM were added HOBt, EDC, DIEA, and 2-morpholin-4-yl-propylamine in that order. The reaction mixture was stirred for 3 hours at room temperature.

The reaction mixture was subject to prep-HPLC purification to afford the intermediate A5a (219 mg).

### Preparation of Compound 5

| | | | | | |
|---|---|---|---|---|---|
| | Chemicals/Reagents | | | | |
| S. No. | & Solvents | MW | mmol | Eq. | Amts |

| | | | |
|---|---|---|---|
| 1 | A5a | 210 | mg |
| 2 | HCl in MeOH | 4 | mL |

Cold solution in MeOH was added to the residue of a. The reaction mixture was stirred for 3 hours at room temperature.

After the reaction was complete (LC-MS), the mixture was purified by a prep-HPLC and converted to an HCl salt by a treatment with HCl in MeOH to afford final compound 5 (120 mg). Compound 5 was characterized by ¹H NMR, LC-MS and HPLC. ¹H NMR (D₂O), δ: 3.30-4.24 (m, 12H), 1.92-2.05 (m, 1H), 1.43-1.56 (m, 1H), 1.36-1.42 (m, 3H), 1.16-1.30 (m, 1H), 1.01 (d, J=6.92 Hz, 3H), 0.94 (d, J=7.34 Hz, 3H). LC-MS, (M+1), 258. HPLC (>95%, retention time, 1.41 min).

### Example 6: Preparation of Compound 6

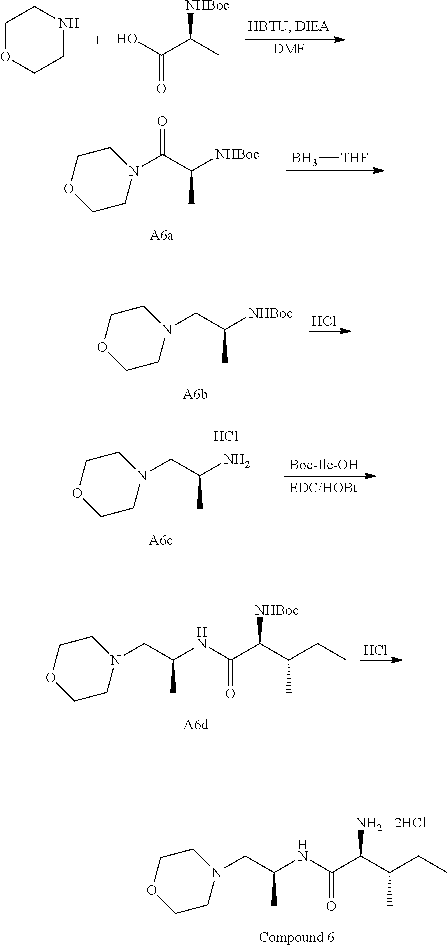

### Preparation of Intermediate A6a

| | Chemicals/Reagents | | | | | |
|---|---|---|---|---|---|---|
| S. No. | & Solvents | MW | mmol | Eq. | Amts | |
| 1 | Boc-Ala-OH | 189.21 | 2 | 1.0 | 378 | mg |
| 2 | DMF (anhydrous) | | | | 5 | mL |
| 3 | DIEA, d = 0.742 | 129.25 | 5 | 2.5 | 0.87 | mL |
| 4 | HBTU | 379 | 2 | 1.0 | 758 | mg |
| 5 | Morpholine, d = 0.99 | 87.12 | 2 | 1.0 | 175 | uL |

To a solution of Boc-Ala-OH in DMF were added HBTU, DIEA, and morpholine in that order. The reaction mixture was stirred for 2 hours at room temperature.

After the reaction was complete (LC-MS), the reaction mixture was subject to prep-HPLC purification to afford the intermediate A6a (333 mg).

### Preparation of Intermediate A6b

| | Chemicals/Reagents | | | | | |
|---|---|---|---|---|---|---|
| S. No. | & Solvents | MW | mmol | Eq. | Amts | |
| 1 | A6a | 258.31 | 1.67 | 1.0 | 430 | mg |
| 2 | THF, anhydrous | | | | 10 | mL |
| 3 | BH₃-THF, 1M | | | 2.0 | 3.3 | mL |

A6a was dissolved in THF. BH₃-THF was added slowly to the above solution. Bubbles were observed and the reaction mixture was stirred over night at RT.

After the reaction was complete (LC-MS), the mixture was quenched carefully with water and concentrated. The mixture was further purified by prep-HPLC to afford intermediate A6b (192 mg).

### Preparation of Intermediate A6c

| | | | | | |
|---|---|---|---|---|---|
| | Chemicals/Reagents | | | | |
| S. No. | & Solvents | MW | mmol | Eq. | Amts |

| | | |
|---|---|---|
| 1 | A6b | 210 mg |
| 2 | HCl in MeOH | 4 mL |

Cold HCl solution in MeOH was added to the residue of A6b. The reaction mixture was stirred for 3 hours at room temperature.

After the reaction was complete (LC-MS), the mixture was concentrated to afford the intermediate A6c (158 mg).

### Preparation of Intermediate A6d

| | Chemicals/Reagents | | | | | |
|---|---|---|---|---|---|---|
| S. No. | & Solvents | MW | mmol | Eq. | Amts | |
| 1 | Boc-Ile-OH | 231.29 | 0.8 | 1.0 | 180 | mg |
| 2 | DMF (anhydrous) | | | | 3 | mL |
| 3 | DIEA, d = 0.742 | 129.25 | 1.6 | 2.0 | 0.28 | mL |
| 4 | HOBt•H₂O | 135.13 | 0.8 | 1.0 | 108 | mg |
| 5 | EDC | 191.7 | 0.8 | 1.0 | 153 | mg |
| 6 | A6c | 180 | 0.83 | | 150 | mg |

To the solution of Boc-Ile-H and A6c in DCM were added HOBt, EDC, and DIEA in that order. The reaction mixture was stirred for 1 hour at room temperature.

The reaction mixture was subject to prep-HPLC purification to afford the intermediate A6d (86 mg).

### Preparation of Compound 6

| | | | | | |
|---|---|---|---|---|---|
| | Chemicals/Reagents | | | | |
| S. No. | & Solvents | MW | mmol | Eq. | Amts |

| | | |
|---|---|---|
| 1 | A6d | 60 mg |
| 2 | 4M HCl in H₂O | 4 mL |

Cold TFA/DCM was added to the residue of A6d. The reaction mixture was stirred for 3 hours at room temperature.

After the reaction was complete (LC-MS), the mixture was concentrated to afford the final compound 6 (89 mg). Compound 6 was characterized by ¹H NMR, LC-MS and HPLC.

¹H NMR (D₂O), δ: 4.21-4.32 (m, 1H), 3.88-4.11 (m, 2H), 3.65-3.87 (m, 3H), 3.34-3.53 (m, 2H), 3.07-3.34 (m, 4H), 1.83-1.95 (m, 1H), 1.30-1.41 (m, 1H), 1.21 (d, J=6.80 Hz, 3H), 1.04-1.17 (m, 1H), 0.91 (d, J=6.96 Hz, 3H), 0.82 (t, J=7.38 Hz, 3H). LC-MS, (M+1), 258. HPLC (>95%, retention time, 1.59 min).

### Example 7: Preparation of Compound 7

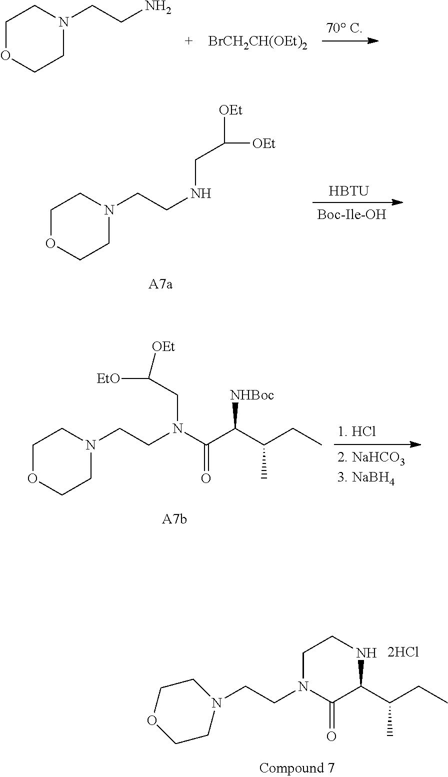

### Preparation of Intermediate A7a

| | Chemicals/Reagents | | | | | |
|---|---|---|---|---|---|---|
| S. No. | & Solvents | MW | mmol | Eq. | Amts | |
| 1 | 2-Morpholin-4-yl-ethylamine | 130.11 | 30 | 3 | 4 | mL |
| 2 | BrCH₂CH(OEt)₂ | 197.07 | 10 | 1 | 1.5 | mL |

Amine and bromide were mixed and heated up to 70° C. for 5 hours with stirring.

LC-MS showed that the product contains a mixture of non-mono-, and bis-alkylated amines. The mixture was used in next step without further purification (5.5 ml).

### Preparation of Intermediate A7b

| | Chemicals/Reagents | | | | | |
|---|---|---|---|---|---|---|
| S. No. | & Solvents | MW | mmol | Eq. | Amts | |
| 1 | Boc-Ile-OH | 231.29 | 30 | 1.0 | 7 | g |
| 2 | A7a | | | | 5.5 | mL |
| 2 | Acetonitrile (anhydrous) | | | | 100 | mL |
| 3 | DIEA, d = 0.742 | 129.25 | 75 | 2.5 | 13 | mL |
| 4 | HBTU | 379.3 | 30 | 1.0 | 11.4 | g |

HBTU was added to a solution of Boc-Ile-OH in acetonitrile and then DIEA was added. After the mixture was stirred at room temperature for 10 minutes, A7a was then added. The reaction mixture was stirred for another 2 hours.

After the reaction was complete (LC-MS), portions of solution were subjected to prep-HPLC separation, collecting fraction peak with M⁺=460 (A7b, 2 g).

### Preparation of Compound 7

A7b was dissolved in cold HCl in MeOH. The reaction mixture was stirred 3 hrs at RT. After the reaction was complete (LC-MS), the mixture was concentrated to afford an HCl salt intermediate (209 mg).

The above HCl salt intermediate was dissolved in a mixed solvent of acetonitrile and water, neutralized with saturated sodium bicarbonate till pH-7. The reaction mixture was stirred for 2.5 hrs at RT. Then, excess NaBH₄ was added and the mixture was stirred overnight. However, not much product was observed by LC-MS the second day morning, thus, the reaction mixture was heated to 45° C. for 7.5 hrs.

After the reaction was complete (LC-MS), the mixture was filtered and subject to prep-HPLC purification to afford the desired final compound. The final compound was converted to an HCl salt by treatment with HCl in MeOH (43 mg). Compound 7 was characterized by ¹H NMR, LC-MS and HPLC. ¹H NMR (D₂O), δ: 2.90-4.20 (m, 17H), 1.84-2.35 (m, 1H), 1.07-1.53 (m, 2H), 0.80-1.02 (m, 6H). LC-MS, (M+1), 270. HPLC (>95%, retention time, 1.27 min).

### Example 8: Preparation of Compound 8

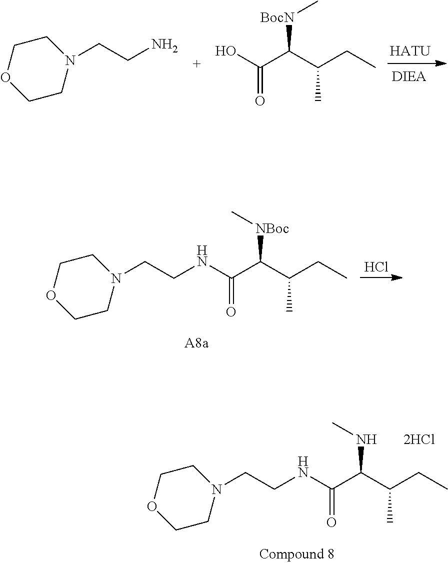

### Preparation of Intermediate A8a

| | Chemicals/Reagents | | | | | |
|---|---|---|---|---|---|---|
| S. No. | & Solvents | MW | mmol | Eq. | Amts | |
| 1 | N-methyl-Boc-Ile-OH | 245.32 | 2 | 2 | 490 | mg |
| 2 | 2-morpholin-4-yl-ethylamine | 130.19 | 1 | 1 | 0.13 | mL |
| 3 | Acetonitrile (anhydrous) | | | | 10 | mL |
| 4 | DIEA, d = 0.742 | 129.25 | 3 | 3 | 0.52 | mL |
| 5 | HATU | 380.2 | 2 | 2 | 760 | mg |

To a solution of acid and HATU solution in acetonitrile, DIEA was added with stirring. After 20 minutes, amine was added and the reaction was continued to stir for 1 hour.

LC-MS showed the completion of the reaction. The reaction solution was subjected to prep-HPLC separation (A8a, 65 mg).

### Preparation of Compound 8

| | Chemicals/Reagents | | | | |
|---|---|---|---|---|---|
| S. No. | & Solvents | MW | mmol | Eq. | Amts |
| 1 | A8a | 357.49 | 0.18 | 1 | 65 mg |
| 2 | Methanol | | | | 10 mL |
| 3 | 12N HCl | | | | 5 mL |

To a solution of A8a in methanol, HCl aqueous solution was added and stirred at room temperature for 2 hours.

LC-MS showed the completion of the reaction. The solvents were removed in vacuum to afford compound 8 (60 mg). Compound 8 was characterized by ¹H NMR, LC-MS and HPLC. ¹H NMR (MeOD), δ: 3.82-4.11 (m, 5H), 3.78 (d, J=4.52 Hz, 1H), 3.68 (d, J=11.2 Hz, 1H), 3.45-3.59 (m, 2H), 3.11-3.41 (m, 5H), 2.78 (s, 3H), 1.93-2.05 (m, 1H), 1.56-1.68 (m, 1H), 1.15-1.32 (m, 1H)), 0.96-1.09 (m, 6H). LC-MS, (M+1), 258. HPLC (>95%, retention time, 1.0 min).

### Example 9: Preparation of Compound 9

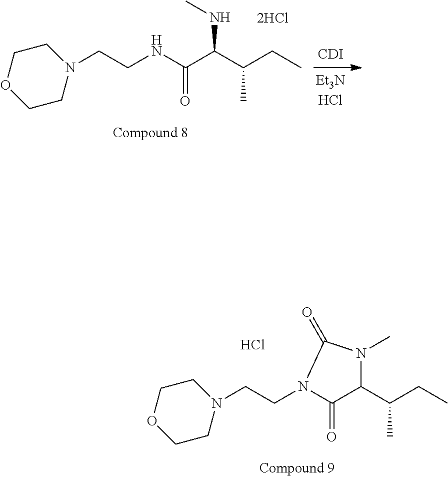

| | Chemicals/Reagents | | | | | |
|---|---|---|---|---|---|---|
| S. No. | & Solvents | MW | mmol | Eq. | Amts | |
| 1 | Compound 8 | 330.29 | 2.27 | 1 | 750 | mg |
| 2 | CDI | 160.17 | 20 | 8.8 | 3.2 | g |
| 3 | Acetonitrile (anhydrous) | | | | 15 | mL |
| 4 | Et₃N | 101.19 | 19.7 | 8.7 | 2 | g |

To a solution of Compound 8 and CDI in acetonitrile, Et₃N was added with stirring. The stirring was continued for three (3) days.

LC-MS showed the completion of the reaction. Methanol was added to quench the reaction. After all the volatile solvent was removed, the residue was redissolved in methanol and subjected to prep-HPLC separation. After removal of the solvent, the product formic acid salt was converted to HCl salt by co-evaporating with 25 ml 1.25 N HCl methanol solution three times (76 mg). Compound 9 was characterized by ¹H NMR, LC-MS and HPLC. ¹H NMR (D₂O), δ: 3.68-4.14 (m, 7H), 3.00-3.63 (m, 6H), 2.68 (d, J=8.40 Hz, 3H), 1.91-2.03 (m, 1H), 0.98-1.51 (m, 2H), 0.90 (d, J=7.00 Hz, 1H), 0.79-0.88 (m, 3H), 0.71 (d, J=6.92 Hz, 1H).

LC-MS, (M+1), 284. HPLC (>95%, retention time, 4.32 min).

### Example 10: Preparation of Compound 10

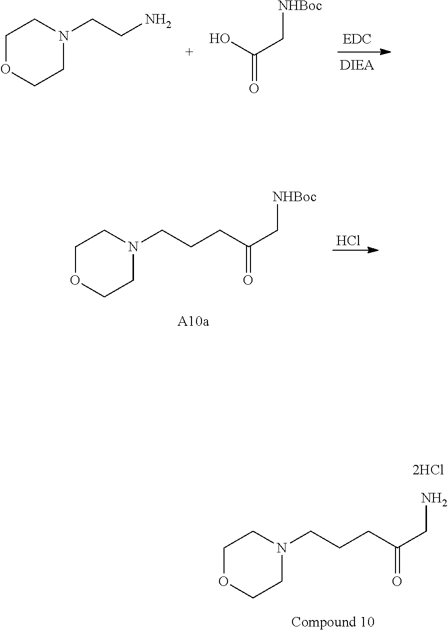 [SMILES: NCCN1CCOCC1 ; CC(C)(C)OC(=O)NCC(=O)O ; CC(C)(C)OC(=O)NCC(=O)CCCN1CCOCC1 ; Cl ; NCC(=O)CCCN1CCOCC1]

### Preparation of Intermediate A10a

| | Chemicals/Reagents | | | | | |
|---|---|---|---|---|---|---|
| S. No. | & Solvents | MW | mmol | Eq. | Amts | |
| 1 | Boc-Gly-OH | 175.18 | 3 | 1 | 526 | mg |
| 2 | 2-morpholin-4-yl-ethylamine | 130.19 | 3 | 1 | 0.39 | mL |
| 3 | Acetonitrile (anhydrous) | | | | 10 | mL |
| 4 | DIEA, d = 0.742 | 129.25 | 6 | 2 | 1.2 | mL |
| 5 | EDC | 191.7 | 4.17 | 1.4 | 800 | mg |

A mixture of Boc-Gly-OH and EDC was suspended in acetonitrile. DIEA was added, followed by the addition of 2-Morpholino-4-yl-ethylamine. The reaction mixture was stirred for 2 hours.

LC-MS showed the completion of the reaction. The reaction mixture was subjected to prep-HPLC separation to give pure A10a (177 mg).

### Preparation of Compound 10

| | Chemicals/Reagents | | | | |
|---|---|---|---|---|---|
| S. No. | & Solvents | MW | mmol | Eq. | Amts |
| 1 | A10a | 357.49 | 0.18 | 1 | 177 mg |
| 2 | Methanol | | | | 50 mL |
| 3 | 12N HCl | | | | 25 mL |

To a solution of A10a in methanol, HCl aqueous solution was added and stirred at room temperature for 2 hours.

LC-MS showed the completion of the reaction. The solvents were removed in vacuum to afford Compound 10 (160 mg). Compound 10 was characterized by ¹H NMR, LC-MS and HPLC. ¹H NMR (MeOD), δ: 3.93-4.08 (m, 4H), 3.77 (s, 2H), 3.56-3.72 (m, 4H), 3.32-3.36 (m, 3H), 3.09-3.24 (m, 2H). LC-MS, (M+1), 188. HPLC (>95%, retention time, 1.0 min).

### Example 11: Preparation of Compound 11

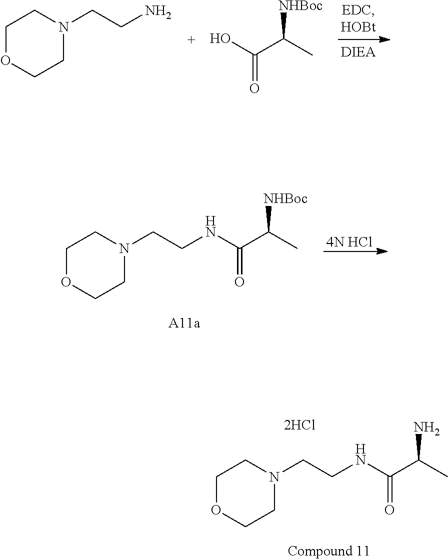

### Preparation of Intermediate A11a

| | Chemicals/Reagents | | | | | |
|---|---|---|---|---|---|---|
| S. No. | & Solvents | MW | mmol | Eq. | Amts | |
| 1 | Boc-Ala-OH | 189.21 | 1 | 1 | 190 | mg |
| 2 | 2-morpholin-4-yl-ethylamine | 130.19 | 1 | 1 | 0.13 | mL |
| 3 | Acetonitrile (anhydrous) | | | | 5 | mL |
| 4 | BtOH•H₂O | 153.14 | 1 | 1 | 153 | mg |
| 5 | DIEA, d = 0.742 | 129.25 | 2 | 2 | 0.38 | mL |
| 6 | EDC | 191.7 | 1 | 1 | 192 | mg |

A mixture of Boc-Ala-OH, BtOH*H₂O, and DIEA was suspended in acetonitrile. EDC was then added with stirring and the solution became clear. 2-Morpholin-4-yl-ethylamine was added and stirred for 2 hours at room temperature.

LC-MS showed the completion of the reaction. The reaction mixture was subjected to prep-HPLC separation to give pure A11a (66 mg).

### Preparation of Compound 11

| | Chemicals/Reagents | | | | |
|---|---|---|---|---|---|
| S. No. | & Solvents | MW | mmol | Eq. | Amts |
| 1 | A11a | 301.38 | 0.22 | 1 | 66 mg |
| 2 | Methanol | | | | 50 mL |
| 3 | 12N HCl | | | | 25 mL |

To a solution of A11a in methanol, HCl aqueous solution was added and stirred at room temperature for 2 hours.

LC-MS showed the completion of the reaction. The solvents were removed in vacuo to afford Compound 11 (60 mg). Compound 11 was characterized by ¹H NMR, LC-MS and HPLC. ¹H NMR (D₂O), δ: 4.10-4.27 (m, 3H), 3.87-4.01 (m, 2H), 3.77-3.88 (m, 1H), 3.60-3.76 (m, 3H) 3.40-3.55 (m, 2H), 3.26-3.39 (m, 2H), 1.60 (d, J=7.16 Hz, 3H). LC-MS, (M+1), 202. HPLC (>95%, retention time, 1.0 min).

### Example 12: Preparation of Compound 12

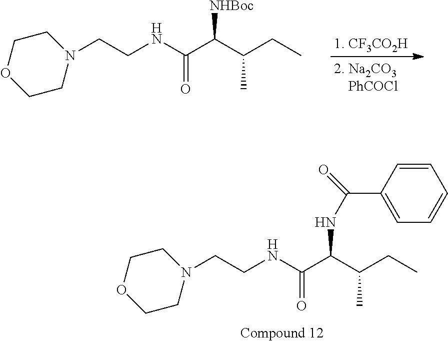 [SMILES: CC[C@H](C)[C@H](NC(=O)OC(C)(C)C)C(=O)NCCN1CCOCC1 ; CC[C@H](C)[C@H](NC(=O)c1ccccc1)C(=O)NCCN1CCOCC1]

| | Chemicals/Reagents | | | | | |
|---|---|---|---|---|---|---|
| S. No. | & Solvents | MW | mmol | Eq. | Amts | |
| 1 | 2-Boc-amino-3-methyl-N-(2- | 343.46 | 1 | 1 | 343 | mg |
| | morpholinoethyl)pentamide | | | | | |
| 2 | CF₃CO₂H | | | | 10 | mL |
| 3 | CH₂Cl₂ | | | | 10 | mL |
| 4 | THF | | | | 10 | mL |
| 5 | Water | | | | 10 | mL |
| 6 | Na₂CO₃ | 105.99 | 10 | 10 | 1.06 | g |
| 7 | PhCOCl | 140.57 | 2 | 2 | 280 | mg |

2-Boc-amino-3-methyl-N-(2-morpholinoethyl)pentamide was dissolved in CF₃CO₂H/CH₂Cl₂ and stirred at room temperature for 2 hours. After LC-MS showed the complete deprotection of the starting material, the solvents were removed under vacuum. The residue was dissolved in THF/H₂O solution and Na₂CO₃ was added to adjust the solution to basic. Benzoyl chloride was then added with stirring for 2 hours.

LC-MS showed the completion of the reaction. THF was then removed under vacuum and the aqueous solution was extracted with AcOEt. AcOEt was dried over Na₂SO₄ and removed. The residue was dissolved in methanol and subjected to prep-HPLC separation. After removal of the solvent, the product formic acid salt was converted to HCl salt by co-evaporating with 50 mL 1.25 HCl methanol solution three times (91 mg). Compound 12 was characterized by ¹H NMR, LC-MS and HPLC. ¹H NMR (MeOD), δ: 7.86-7.91 (m, 2H), 7.56-7.61 (m, 1H), 7.47-7.53 (m, 2H), 4.20 (d, J=7.96 Hz, 1H), 4.00-4.10 (m, 2H), 3.82-3.92 (m, 2H), 3.71-3.80 (m, 1H), 3.59-3.69 (m, 2H), 3.15-3.56 (m, 5H), 1.96-2.08 (m, 1H), 1.63-1.75 (m, 1H), 1.26-1.39 (m, 1H), 0.94-1.06 (m, 6H). LC-MS, (M+1), 348. HPLC (>95%, retention time, 5.27 min).

### Example 13: Preparation of Compound 13

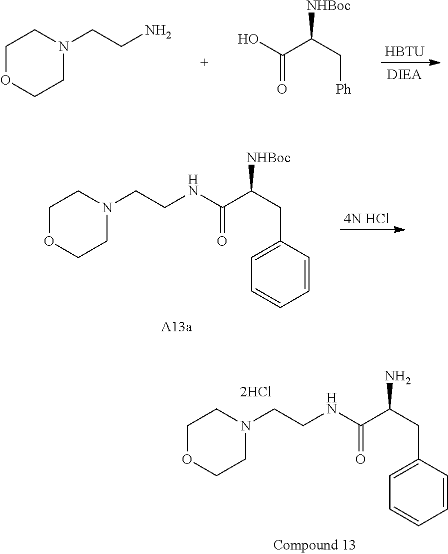

### Preparation of Intermediate A13a

| | Chemicals/Reagents | | | | | |
|---|---|---|---|---|---|---|
| S. No. | & Solvents | MW | mmol | Eq. | Amts | |
| 1 | Boc-Phe-OH | 256.31 | 1 | 1 | 256 | mg |
| 2 | 2-morpholin-4-yl-ethylamine | 130.19 | 1 | 1 | 0.13 | mL |
| 3 | Acetonitrile (anhydrous) | | | | 5 | mL |
| 4 | DIEA, d = 0.742 | 129.25 | 1 | 1 | 0.17 | mL |
| 5 | HBTU | 379.25 | 1 | 1 | 379 | mg |

To a solution of Boc-Pe-H, HBTU in acetonitrile was added DIEA. After stirring for 5 minutes, 2-morpholin-4-yl-ethylamine was added and the reaction mixture was then stirred for another 2 hours.

LC-MS showed the completion of the reaction. The reaction mixture was subjected to prep-HPLC separation to give pure A13a (210 mg).

### Preparation of Compound 13

| | Chemicals/Reagents | | | | |
|---|---|---|---|---|---|
| S. No. | & Solvents | MW | mmol | Eq. | Amts |
| 1 | A13a | 377.48 | 0.56 | 1 | 210 mg |
| 2 | Methanol | | | | 50 mL |
| 3 | 12N HCl | | | | 25 mL |

To a solution of A13a in methanol, HCl aqueous solution was added and stirred at room temperature for 2 hours.

LC-MS showed the completion of the reaction. The solvents were removed in vacuum to afford Compound 13 (195 mg). Compound 13 was characterized by ¹H NMR, LC-MS and HPLC. ¹H NMR (D₂O), δ: 7.32-7.41 (m, 2H), 7.43-7.57 (m, 3H), 4.30 (t, J=7.32 Hz, 1H), 3.94-4.10 (m, 4H), 3.61 (t, J=6.34 Hz, 2H), 3.08-3.44 (m, 9H). LC-MS, (M+1), 278. HPLC (>95%, retention time, 1.21 min).

A number of compounds, including Compound 14, Compound 15, Compound 16, Compound 17, and Compound 18, were prepared according to the method of preparation of Compound 13, substituting the appropriate starting materials in place of Boc-Phe-OH.

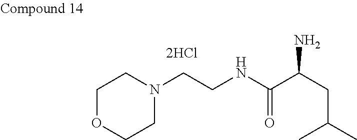 [SMILES: CC(C)C[C@H](N)C(=O)NCCN1CCOCC1]

Compound 14 was prepared according to the method of preparation of Compound 13, except that Boc-Phe-OH was replaced with Boc-Leu-OH. Compound 14 (175 mg) was characterized by ¹H NMR, LC-MS and HPLC. ¹H NMR (D₂O), δ: 4.07-4.20 (m, 2H), 4.03 (d, J=7.22 Hz, 1H), 3.71-3.93 (m, 3H), 3.50-3.70 (m, 3H), 3.18-3.44 (m, 4H), 1.74 (t, J=7.26 Hz, 2H), 1.61-1.72 (m, 1H), 0.89-1.03 (m, 6H). LC-MS, (M+1), 244. HPLC (>95%, retention time, 1.0 min).

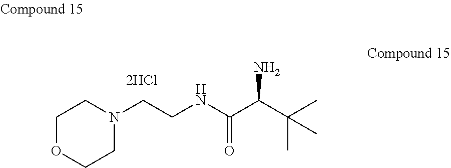 [SMILES: CC(C)(C)[C@H](N)C(=O)NCCN1CCOCC1]

Compound 15 was prepared according to the method of preparation of Compound 13, except that Boc-Phe-OH was replaced with Boc-D-t-butylglycine-OH. Compound 15 (120 mg) was characterized by ¹H NMR, LC-MS and HPLC. ¹H NMR (D₂O), δ: 3.91-4.10 (m, 2H), 3.62-3.82 (m, 3H), 3.60 (s, 1H), 3.38-3.58 (m, 3H), 3.05-3.34 (m, 4H), 0.96 (s, 9H). LC-MS, (M+1), 244. HPLC (>95%, retention time, 1.0 min).

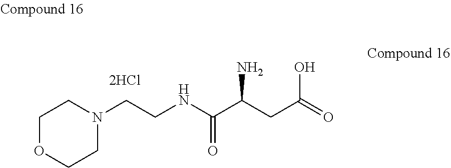 [SMILES: N[C@@H](CC(=O)O)C(=O)NCCN1CCOCC1]

Compound 16 was prepared according to the method of preparation of Compound 13, except that Boc-Phe-OH was replaced with Boc-Asp(OTBU)—OH. Compound 16 (50 mg) was characterized by ¹H NMR, LC-MS and HPLC. ¹H NMR (D₂O), δ: 4.23 (t, J=6.24 Hz, 1H), 3.91-4.11 (m, 2H), 3.67-3.84 (m, 2H), 3.55-3.67 (m, 2H), 3.41-3.54 (m, 2H), 3.27 (t, J=6.32 Hz, 2H), 3.06-3.22 (m, 2H), 2.85-3.01 (m, 2H). LC-MS, (M+1), 246. HPLC (>95%, retention time, 1.0 min).

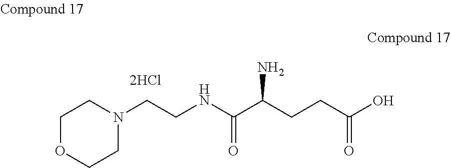 [SMILES: N[C@@H](CCC(=O)O)C(=O)NCCN1CCOCC1]

Compound 17 was prepared according to the method of preparation of Compound 13, except that Boc-Phe-OH was replaced with Boc-Glu(OTBU)—OH. Compound 17 (60 mg) was characterized by ¹H NMR, LC-MS and HPLC. ¹H NMR (D₂O), δ: 3.94-4.10 (m, 2H), 3.97 (t, J=6.56 Hz, 1H), 3.67-3.83 (m, 2H), 3.60-3.69 (m, 1H), 3.40-3.59 (m, 3H), 3.20 (t, J=6.78 Hz, 2H), 3.08-3.23 (m, 2H), 2.45 (t, J=7.18 Hz, 2H), 2.01-2.17 (m, 2H). LC-MS, (M+1), 260.

HPLC (>95%, retention time, 1.0 min).

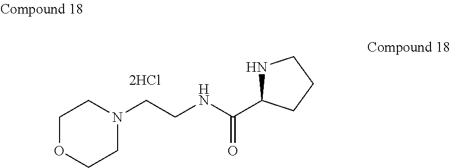 [SMILES: O=C(NCCN1CCOCC1)[C@@H]1CCCN1]

Compound 18 was prepared according to the method of preparation of Compound 13, except that Boc-Phe-OH was replaced with Boc-Pro-OH. Compound 18 (80 mg) was characterized by ¹H NMR, LC-MS and HPLC. ¹H NMR (D₂O), δ: 4.27 (t, J=7.38 Hz, 1H), 3.91-4.10 (m, 2H), 3.60-3.81 (m, 3H), 3.37-3.57 (m, 3H), 3.19-3.37 (m, 4H), 3.04-3.19 (m, 2H), 2.25-2.40 (m, 1H), 1.85-2.02 (m, 3H). LC-MS, (M+1), 228. HPLC (>95%, retention time, 1.0 min).

### Example 19: Preparation of Compound 19

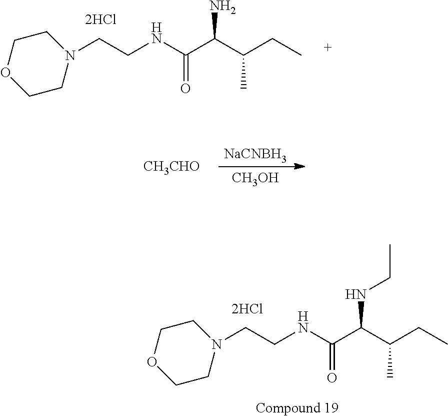 [SMILES: CC[C@H](C)[C@H](N)C(=O)NCCN1CCOCC1 ; "NaCNBH3" ; "CH3CHO" ; "CH3OH" ; CCN[C@H](C(=O)NCCN1CCOCC1)[C@@H](C)CC]

| S. | | | | | |
|---|---|---|---|---|---|
| No. | Chemicals/Reagents & Solvents | MW | mmol | Eq. | Amts |
| 1 | (2S,3S)-2-amino-3-methyl-N-(2- | 316.27 | 1 | 1 | 314 mg |
| | morpholinoethyl)pentanamide | | | | |
| 2 | CH₃CHO | 44.05 | 1 | 1 | 44 mg |
| 3 | NaCNBH₃ (1M in THF) | | 1 | 1 | 1 mL |
| 4 | Methanol | 105.99 | 10 | 10 | 1.06 g |

A mixture of (2S,3 S)-2-amino-3-methyl-N-(2-morpholinoethyl)pentanamide and acetaldehyde was dissolved in methanol and NaCNBH₃ was added. The reaction mixture was then stirred overnight.

LC-MS showed the completion of the reaction. K₂CO₃ (270 mg) was added to quench the reaction. The precipitate was dissolved by adding 2 mL water and then the solution was subjected to prep-HPLC separation. Then the formic acid salt was transformed to HCl salt by co-evaporating with HCl/methanol (1N) solution. Compound 19 (50 mg) was characterized by ¹H NMR and LC-MS. ¹H NMR (D₂O), δ: 4.05-4.23 (m, 2H), 3.48-3.94 (m, 7H), 3.38 (t, J=6.82 Hz, 2H), 3.17-3.34 (m, 2H), 3.08 (q, J=7.32 Hz, 2H), 1.94-2.07 (m, 1H), 1.44-1.60 (m, 1H), 1.30 (t, J=7.32 Hz, 1H), 1.14-1.26 (m, 3H), 0.89-1.06 (m, 6H). LC-MS, (M+1), 272.

### Example 20: Preparation of Compound 20

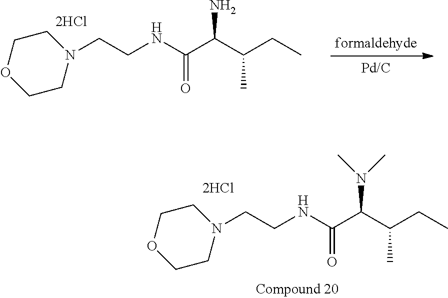 [SMILES: CC[C@H](C)[C@H](N)C(=O)NCCN1CCOCC1 ; CC[C@H](C)[C@@H](C(=O)NCCN1CCOCC1)N(C)C]

| S. | | | | | |
|---|---|---|---|---|---|
| No. | Chemicals/Reagents & Solvents | MW | mmol | Eq. | Amts |
| 1 | (2S,3S)-2-amino-3-methyl-N-(2- | 316.27 | 1 | 1 | 316 mg |
| | morpholinoethyl)pentanamide | | | | |
| 2 | Formaldehyde (37% solution) | 44.05 | 16.8 | 16.8 | 2 mL |
| 3 | Pd/C (10%) | | | | 1 g |
| 4 | H₂O | | | | 10 mL |

A mixture of (2S,3 S)-2-amino-3-methyl-N-(2-morpholinoethyl)pentanamide, formaldehyde, and Pd/C in water was hydrogenated at 50 Psi for 5 hours.

LC-MS showed the completion of the reaction. Pd/C was filtrated off and the clear solution was subjected to prep-HPLC separation. The formic acid salt form was transformed to HCl salt (220 mg) by co-evaporating with HCl/methanol (1N) solution. Compound 20 (220 mg) was characterized by ¹H NMR, LC-MS and HPLC. ¹H NMR (D₂O), δ: 3.70-4.13 (m, 4H), 3.64-3.69 (m, 1H), 3.56-3.64 (m, 2H), 3.05-3.52 (m, 6H), 2.80 (s, 6H), 2.02-2.15 (m, 1H), 1.32-1.46 (m, 1H), 1.01-1.15 (m, 1H), 0.81-0.93 (m, 6H). LC-MS, (M+1), 272.

HPLC (>95%, retention time, 1.14 min).

### Example 21: Preparation of Compound 21

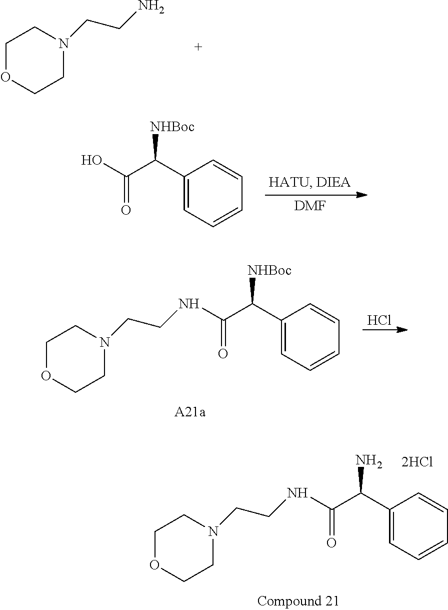

### Preparation of Intermediate A11a

| | Chemicals/ | | | | |
|---|---|---|---|---|---|
| S. No. | Reagents & Solvents | MW | mmol | Eq. | Amts |
| 1 | Boc-L-alpha-phenylglycine | 251.28 | 1 | 1.0 | 251 mg |
| 2 | DMF (anhydrous) | | | | 6 mL |
| 3 | DIEA, d = 0.742 | 129.25 | 2.0 | 2.0 | 0.35 mL |
| 4 | HATU | 380.2 | 1 | 1.0 | 380 mg |
| 5 | Amine, d = 1 | 130.19 | 2 | 2.0 | 0.13 mL |

To a solution of acid in DMF were added HATU DIEA, and amine in that order. The reaction mixture was stirred for 2 hr at room temperature.

After the reaction was complete (LC-MS and TLC), the intermediate A21a was purified by prep-HPLC (228 mg).

### Preparation of Compound 21

| S. No. | Chemicals/Reagents & Solvents | MW | mmol | Eq. | Amts |
|---|---|---|---|---|---|
| 1 | A21a | | | | 228 mg |
| 2 | 4M HCl in MeOH | | | | 4 mL |

Cold HCl solution was added to the residue of A21a. The reaction mixture was stirred for overnight at room temperature.

After the reaction was complete (LC-MS), the mixture was concentrated. It was further purified on prep-HPLC to afford the final compound 21 (206 mg). Compound 21 was characterized by ¹H NMR, LC-MS and HPLC. ¹H NMR (D₂O), δ: 7.34-7.49 (m, 5H), 5.04 (s, 1H), 3.86-3.98 (m, 2H), 3.58-3.72 (m, 2H), 3.54 (t, J=6.22 Hz, 2H), 3.14-3.39 (m, 4H), 2.92-3.07 (m, 2H). LC-MS, (M+1), 264. HPLC (>95%, retention time, 0.98 min).

Preparation of lead analogs of a number of compounds of the present application is illustrated in the general Scheme 2 below:

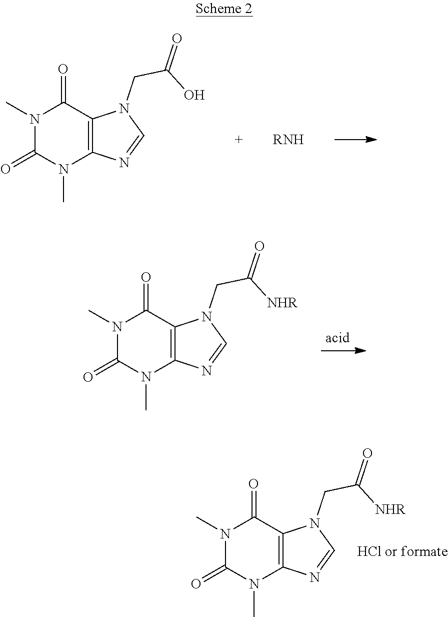

In such a synthesis strategy, the coupling agent can be HATU or HBTU. The acid used to remove a protection group such as Boc can be 4 M HCl in MeOH or 4M HCl in water.

Preparation of additional compounds of the present application can be illustrated in the general Scheme 3 below:

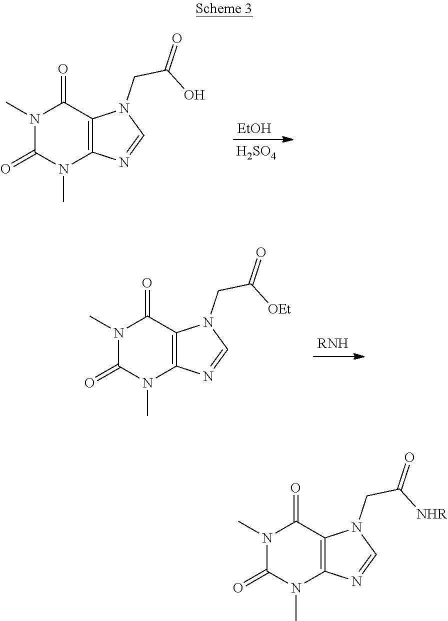

In this synthesis, the starting material acid is first converted to an ester. Then the ester is reacted with an amine to afford an amide compound. The amide compound may undergo further transformation, such as, reductive amination to afford the final compound.

### Example 22: Preparation of Compound 22

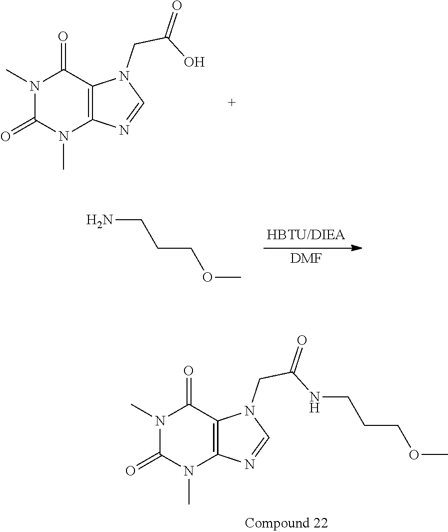

| S. | | | | | |
|---|---|---|---|---|---|
| No. | Chemicals/Reagents & Solvents | MW | mmol | Eq. | Amts |
| 1 | acid | 238.2 | 1 | 1.0 | 238 mg |
| 2 | DCM (anhydrous) | | | | 5 mL |
| 3 | DIEA, d = 0.742 | 129.25 | 2.0 | 2.0 | 0.35 mL |
| 4 | HBTU | 379.25 | 1 | 1.0 | 379 mg |
| 5 | Amine, d = 0.871 | 89.14 | 1 | 1.0 | 0.1 mL |

To a solution of acid in DMF were added HBTU, DIEA, and amine in that order. The reaction mixture was stirred over night at room temperature.

After the reaction was complete (LC-MS and TLC), brine and saturated sodium bicarbonate were added to quench the reaction. 10% MeOH in DCM was used to extract the aqueous layer (3X). The DCM layer was separated, combined and washed with brine, dried with anhydrous Na₂SO₄, filtered, and concentrated. The Compound 22 was obtained after silica gel column chromatography as an off-white solid (138 mg). Compound 22 was characterized by ¹H NMR, LC-MS and HPLC. ¹H NMR (D₂O), δ: 7.90 (s, 2H), 5.00 (s, 2H), 3.46 (s, 3H), 3.40 (t, J=6.40 Hz, 2H), 3.19-3.28 (m, 8H), 1.65-1.75 (m, 2H). LC-MS, (M+1), 310. HPLC (>95%, retention time, 5.76 min).

### Example 23: Preparation of Compound 23

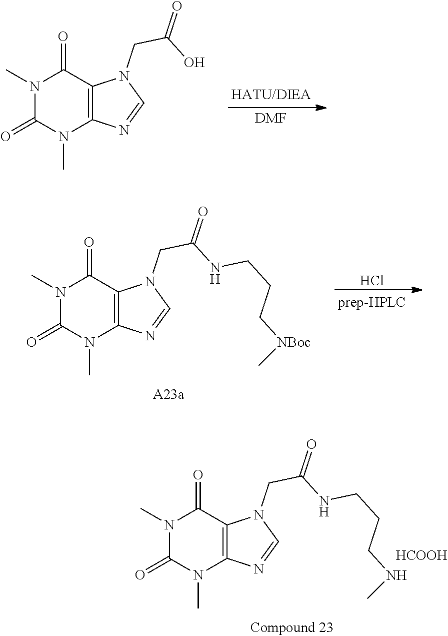

### Preparation of Intermediate A23a

| S. | | | | | |
|---|---|---|---|---|---|
| No. | Chemicals/Reagents & Solvents | MW | mmol | Eq. | Amts |
| 1 | acid | 238.2 | 1 | 1.0 | 238 mg |
| 2 | DMF (anhydrous) | | | | 5 mL |
| 3 | DIEA, d = 0.742 | 129.25 | 2.0 | 2.0 | 0.35 mL |
| 4 | HATU | 380.2 | 1 | 1.0 | 380 mg |
| 5 | Amine | 188.271 | 1 | 1.0 | 0.2 mL |

To a solution of acid in DMF were added HATU, DIEA, and amine in that order. The reaction mixture was stirred for 1 hr at room temperature.

After the reaction was complete (LC-MS and TLC), brine and saturated sodium bicarbonate were added to quench the reaction. 5% MeOH in DCM was used to extract the aqueous layer (3X). The DCM layer was separated, combined and washed with brine, dried with anhydrous Na₂SO₄, filtered, and concentrated. The compound was further purified by prep-HPLC (150 mg).

### Preparation of Compound 23

| S. No. | Chemicals/Reagents & Solvents | MW | mmol | Eq. | Amts |
|---|---|---|---|---|---|
| 1 | A23a | | | | 145 mg |
| 2 | 4M HCl in H₂O | | | | 4 mL |

Cold HCl solution was added to the residue of C9a. The reaction mixture was stirred for 3 hours at room temperature.

After the reaction was complete (LC-MS), the mixture was concentrated. It was further purified on prep-HPLC to afford the final compound 23 (73 mg). Compound 23 was characterized by ¹H NMR, LC-MS and HPLC. ¹H NMR (D₂O), δ: 7.89 (s, 1H), 4.99 (s, 2H), 3.45 (s, 3H), 3.25 (t, J=6.58 Hz, 2H), 3.22 (s, 3H), 2.96 (t, J=7.66 Hz, 2H), 2.60 (s, 3H), 1.76-1.86 (m, 2H). LC-MS, (M+1), 309. HPLC (>95%, retention time, 1.20 min).

### Example 24: Preparation of Compound 24

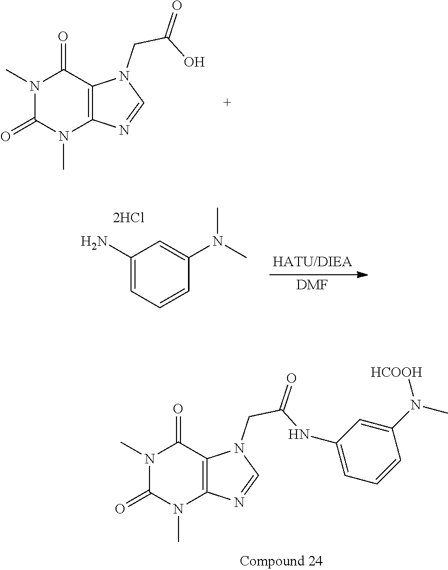

| S. No. | Chemicals/Reagents & Solvents | MW | mmol | Eq. | Amts |
|---|---|---|---|---|---|
| 1 | acid | 238.2 | 1 | 1.0 | 238 mg |
| 2 | DMF (anhydrous) | | | | 8 mL |
| 3 | DIEA, d = 0.742 | 129.25 | 2.0 | 2.0 | 0.7 mL |
| 4 | HATU | 380.2 | 1 | 1.0 | 380 mg |
| 5 | Aniline | 209.12 | 1 | 1.0 | 209 mg |

To a solution of acid in DMF were added HATU, DIEA, and aniline in that order. The reaction mixture was stirred for 2 hr at room temperature.

After the reaction was complete (LC-MS and TLC), the compound was purified by prep-HPLC (250 mg). Compound 24 was characterized by ¹H NMR, LC-MS and HPLC. ¹H NMR (CDCl3), δ: 9.25 (s, 1H), 7.77 (s, 1H), 7.15 (t, J=8.14 Hz, 1H), 7.05 (s, 1H), 6.74-6.80 (m, 1H), 6.46-6.53 (m, 1H), 4.95 (s, 2H), 3.61 (s, 3H), 3.46 (s, 3H), 2.93 (s, 6H), 1.59 (s, 3H).

LC-MS, (M+1), 357. HPLC (>95%, retention time, 5.79 min).

### Example 25: Preparation of Compound 25

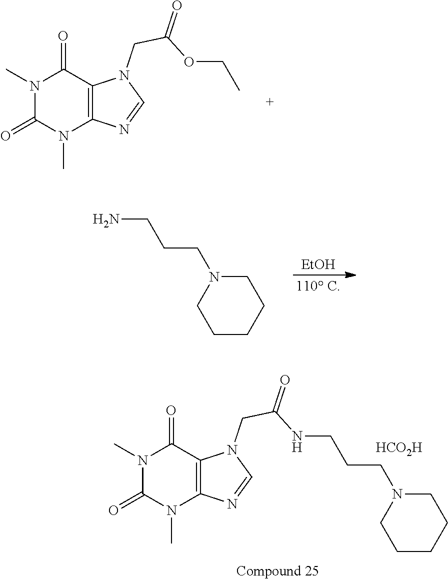

| S. No. | Chemicals/Reagents & Solvents | MW | mmol | Eq. | Amts |
|---|---|---|---|---|---|
| 1 | ester | 266.25 | 2.5 | 1 | 660 mg |
| 2 | 3-piperidin-1-yl-propylamine | 142.24 | 3.5 | 1.4 | 0.5 mL |
| 3 | EtOH | | | | 3 mL |

A mixture of ester (prepared by refluxing 18 g of Theophylline-7-acetic acid in 300 ml anhydrous EtOH with 1 mL concentrated H₂SO₄ as a catalyst) and 3-piperidin-1-yl-propylamine was suspended in anhydrous ethanol and sealed in a high pressure bottle. The reaction mixture was heated up to 110° C. with stirring for 2 hours.

LC-MS showed the completion of the reaction. 10 mL methanol was added and the solution was subjected to prep-HPLC separation (200 mg). Compound 25 was characterized by ¹H NMR, LC-MS and HPLC. ¹H NMR (D₂O), δ: 8.34 (s, 1H), 7.90 (s, 1H), 4.98 (s, 2H), 3.40-3.48 (m, 2H), 3.39 (s, 3H), 3.25 (t, J=6.48 Hz, 2H), 3.17 (s, 3H), 3.00-3.07 (m, 2H), 2.78-2.88 (m, 2H), 1.53-1.93 (m, 7H), 1.31-1.45 (m, 1H). LC-MS, (M+1), 363. HPLC (>95%, retention time, 1.91 min).

### Example 26: Preparation of Compound 26

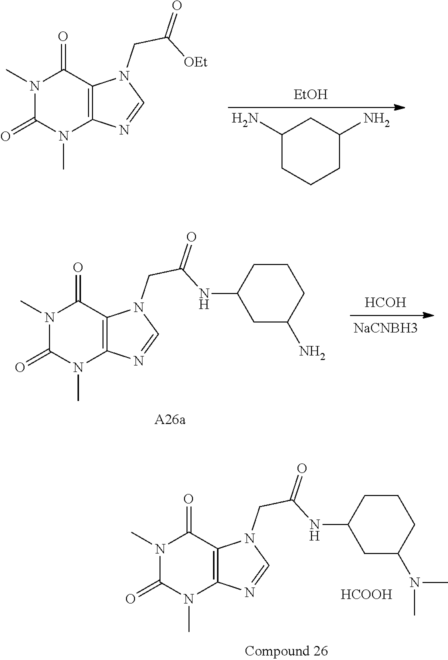

### Preparation of Intermediate A26a

| S. | | | | | |
|---|---|---|---|---|---|
| No. | Chemicals/Reagents & Solvents | MW | mmol | Eq. | Amts |
| 1 | Ester | 266.2 | 2.0 | 1.0 | 532 mg |
| 2 | EtOH, absolute | | | | 10 mL |
| 3 | 1,2 cyclohexane diamine, | 114.19 | 2.25 | 1.12 | 270 uL |
| | d = 0.95 | | | | |

The ester and the diamine were suspended in EtOH. The reaction mixture was refluxed for 2 hours.

The mixture was concentrated and purified on prep-HPLC. The compound was triturated with MeOH-Et₂O four times to remove trace amount of diamine (140 mg).

### Preparation of Compound 26

| S. | | | | | |
|---|---|---|---|---|---|
| No. | Chemicals/Reagents & Solvents | MW | mmol | Eq. | Amts |
| 1 | A26a | 344 | 0.4 | 1.0 | 136 mg |
| 2 | HCOH (aq., 37%) | | | | 4 mL |
| 3 | NaCNBH₃ | 62.84 | 0.82 | 2 | 51 mg |

A26a was dissolved in aqueous HCOH and NaCNBH₃ was added. The reaction mixture was stirred for 2 hours at RT.

The mixture was concentrated. It was purified on prep-HPLC to afford the Compound 26 (97 mg). Compound 26 was characterized by ¹H NMR, LC-MS and HPLC. ¹H NMR (D₂O), δ: 8.31 (s, 1H), 7.89 (s, 1H), 4.98 (s, 2H), 3.66-3.78 (m, 1H), 3.47 (s, 3H), 3.18-3.27 (m, 4H), 2.72-2.80 (m, 6H), 2.17-2.25 (m, 1H), 1.11-2.04 (m, 8H). LC-MS, (M+1), 363.

HPLC (>95%, retention time, 1.95 min).

### Example 27: Preparation of Compound 27

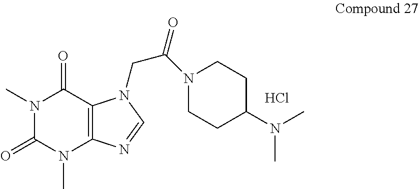 [SMILES: CN(C)C1CCN(C(=O)Cn2cnc3c2c(=O)n(C)c(=O)n3C)CC1 ; Cl]

Compound 27 (180 mg) was prepared by the same method as Compound 26 using an appropriate starting material in place of 1,2-cyclohexane diamine. The only difference was that the reaction was carried out at 130° C. for 6 hours, and the final formic acid salt was converted to HCl salt by co evaporating with 50 mL of 1.25N HCl methanol solution three times. Compound 27 was characterized by ¹H NMR, LC-MS and HPLC. ¹H NMR (D₂O), δ: 7.85 (s, 1H), 5.19-5.38 (m, 3H), 4.41-4.50 (m, 1H), 4.00-4.08 (m, 1H), 3.46-3.53 (m, 1H), 3.45 (s, 3H), 3.18-3.28 (m, 4H), 2.80 (s, 6H), 2.68-2.78 (m, 1H), 2.04-2.21 (m, 2H), 1.73-1.86 (m, 1H), 1.53-1.66 (m, 1H). LC-MS, (M+1), 349. HPLC (>95%, retention time, 1.71 min).

### Example 28: Preparation of Compound 28

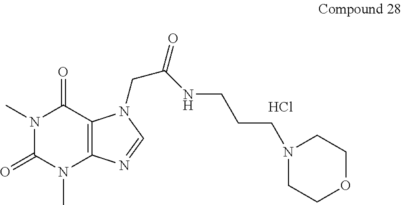 [SMILES: Cn1c(=O)c2c(ncn2CC(=O)NCCCN2CCOCC2)n(C)c1=O ; Cl]

Compound 28 (130 mg) was prepared by the same method as Compound 27 using a derivatized amine in place of 1,2-cyclohexane diamine, and the final formic acid salt was converted to HCl salt by co evaporating with 50 mL 1.25N HCl methanol solution three times. Compound 28 was characterized by ¹H NMR, LC-MS and HPLC. ¹H NMR (D₂O), δ: 7.89 (s, 1H), 4.99 (s, 2H), 3.97-4.05 (m, 2H), 3.64-3.75 (m, 2H), 3.40-3.47 (m, 5H), 3.16-3.29 (m, 7H), 3.03-3.16 (m, 4H), 1.83-1.94 (m, 2H). LC-MS, (M+1), 365. HPLC (>95%, retention time, 1.0 min).

### Example 29: Preparation of Compound 29

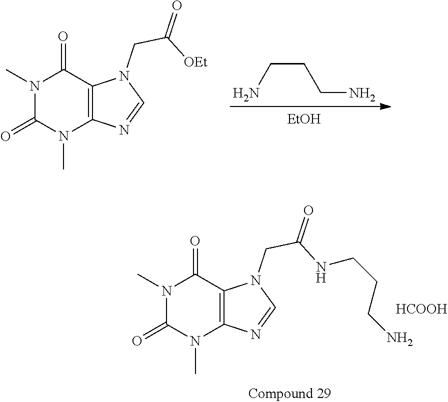 [SMILES: "EtOH" ; CCOC(=O)Cn1cnc2c1c(=O)n(C)c(=O)n2C ; NCCCN ; Cn1c(=O)c2c(ncn2CC(=O)NCCCN)n(C)c1=O ; "HCOOH"]

| S. No. | Chemicals/Reagents & Solvents | MW | mmol | Eq. | Amts |
|---|---|---|---|---|---|
| 1 | Ester | 266.2 | 1.0 | 1.0 | 266 mg |
| 2 | EtOH, absolute | | | | 5 mL |
| 3 | Diamine | 74.13 | 1.0 | 1.0 | 84 uL |

Ester and diamine were suspended in EtOH. The reaction mixture was refluxed for 2 hours.

After the reaction was complete (LC-MS), the mixture was concentrated. It was purified on prep-HPLC to afford Compound 29. The final compound was triturated with MeOH-Et2O four times to remove trace amount of diamine (155 mg). Compound 29 was characterized by ¹H NMR, LC-MS and HPLC. ¹H NMR (D₂O), δ: 8.33 (s, 1H), 7.87 (s, 1H), 4.99 (s, 2H), 3.44 (s, 3H), 3.24 (t, J=6.66 Hz, 2H), 3.20 (s, 3H), 2.91 (t, J=7.62 Hz, 2H), 1.74-1.82 (m, 2H). LC-MS, (M+1), 295. HPLC (>95%, retention time, 1.12 min).

### Example 30: Preparation of Compound 30

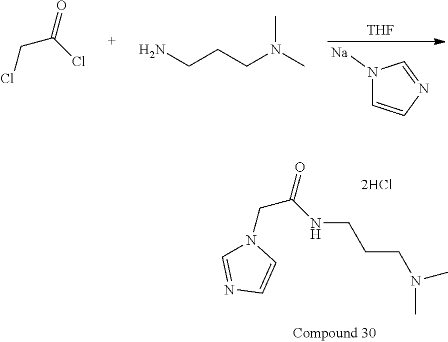

| S. No. | Chemicals/Reagents & Solvents | MW | mmol | Eq. | Amts |
|---|---|---|---|---|---|
| 1 | Chloroacetyl chloride | 112.94 | 10 | 1.0 | 0.8 mL |
| 2 | 3-(Dimethylamino)-1- | 102.18 | 10 | 1.0 | 1.2 mL |
| | propylamine | | | | |
| 3 | imidazole | 68.08 | 10 | 1.0 | 680 mg |
| 4 | NaH (60%) | 24.00 | 10 | 1.0 | 400 mg |
| 5 | THF | | | | 40 mL |

To a solution of chloroacetyl chloride in THF was added 3-(dimethylamino)-1-propylamine at 0° C. with stirring for 20 minutes. LC-MS showed the formation of amide. To this solution, imidazole sodium salt in THF (prepared from imidazole and NaH in 10 mL THF) was added. The reaction was continued for 2 hours with stirring.

After the reaction was complete (LC-MS), THF was removed. The residue was dissolved in methanol and subjected to prep-HPLC separation. The final compound was still not pure at this stage. Then it was purified again by ISCO C18 reverse phase chromatography to give the pure final product. The product was converted to HCl salt by evaporating together with HCl/methanol (1N) solution (100 mg). Compound 30 was characterized by ¹H NMR, LC-MS and HPLC. ¹H NMR (D₂O), δ: 8.71 (s, 1H), 7.36-7.48 (m, 2H), 5.02 (s, 2H), 3.25 (t, J=6.80 Hz, 2H), 3.04-3.10 (m, 2H), 2.78 (s, 6H), 1.83-1.92 (m, 2H). LC-MS, (M+1), 211.

HPLC (>95%, retention time, 1.01 min).

### Example 31: Preparation of Compound 31

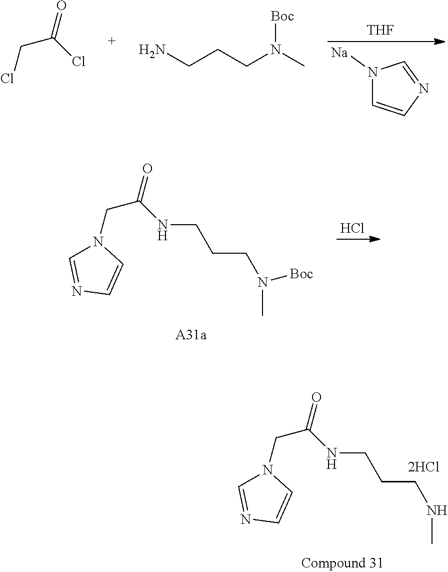

### Preparation of intermediate A31a

Intermediate A31a (250 mg) was prepared similarly to Compound 30.

### Preparation of Compound 31

| S. No. | Chemicals/Reagents & Solvents | MW | mmol | Eq. | Amts |
|---|---|---|---|---|---|
| 1 | A31a | 296.37 | | | 250 mg |
| 2 | Methanol | | | | 50 mL |
| 3 | 12N HCl | | | | 25 mL |

Intermediate A31a was dissolved in methanol and HCl aqueous solution was added and the reaction was stirred for 2 hours.

After the reaction was complete (LC-MS), the mixture was concentrated. The residue was dissolved in water and subjected to prep-HPLC separation. After removal of solvent, the formic acid salt was converted to HCl salt by coevaporating with 50 mL 1.25 HCl methanol solution three times (200 mg). Compound 31 was characterized by ¹H NMR, LC-MS and HPLC. ¹H NMR (D₂O), δ: 8.70 (s, 1H), 7.33-7.46 (m, 2H), 5.01 (s, 2H), 3.25 (t, J=6.84 Hz, 2H) 2.95 (t, J=7.76 Hz, 2H), 2.60 (s, 3H), 1.77-1.87 (m, 2H). LC-MS, (M+1), 197. HPLC (>95%, retention time, 0.99 min).

### Example 32: Preparation of Enantiomerically Pure 2-amino-3-methyl-N-(2-morpholino-ethyl)-pentanamide

2-amino-3-methyl-N-(2-morpholinoethyl)-pentanamide can be prepared by a method shown in Scheme 4 below. First, 2-aminoethanol (Compound 1E) is transformed to its derivative with a leaving group (Compound 2E). Examples of the leaving group include halides and alkoxy or other activated hydroxyl group. Second, Compound 2E reacts with morpholine at a neutral or basic condition to yield 2-morpholinoethanamine (Compound 3E). The aforementioned two steps may also be performed continuously as one step with Compound 2E being generated in situ. For example, Compound 3E can be prepared from Compound 1E directly through a Mitsunobu reaction wherein the hydroxyl group of Compound 1E is activated by diethyl azodicarboxylate (DEAD) before morpholine is added. The final product, 2-amino-3-methyl-N-(2-morpholinoethyl)-pentanamide (Compound 5E), can be obtained by coupling 2-morpholinoethanamine with 2-amino-3-methylpentanoic acid (Compound 4E) via a peptide coupling agent. Examples of the peptide coupling agent include 1,1′-carbonyldiimidazole (CDI), hydroxybenzotriazole (HOBT), 1,3-dicyclohexylcarbodiimide (DCC), 1-hydroxybenzo-7-azatriazole (HOAt), and the like.

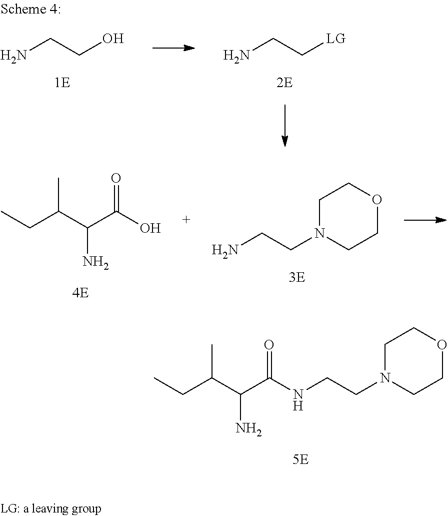

A chiral 2-amino-3-methyl-N-(2-morpholinoethyl)-pentanamide (Compound 5E) can be obtained by using the corresponding chiral 2-amino-3-methylpentanoic acid (Compound 4E) in the above coupling step. For example, (2S,3S)-2-amino-3-methyl-N-(2-morpholinoethyl)-pentanamide; (2R,3R)-2-amino-3-methyl-N-(2-morpholinoethyl)-pentanamide; (2R,3S)-2-amino-3-methyl-N-(2-morpholinoethyl)-pentanamide; and (2S,3R)-2-amino-3-methyl-N-(2-morpholinoethyl)-pentanamide can be obtained by using (2S,3 S)-2-amino-3-methylpentanoic acid, i.e., L-isoleucine; (2R,3R)-2-amino-3-methylpentanoic acid, i.e., D-isoleucine; (2R,3 S)-2-amino-3-methylpentanoic acid, i.e., D-alloisoleucine; and (2S,3R)-2-amino-3-methylpentanoic acid, i.e., L-alloisoleucine, respectively.

The chiral purity, also known as, enantiomeric excess or EE, of a chiral Compound 5E can be determined by any method known to one skilled in the art. For example, a chiral Compound 5E can be hydrolyzed to Compound 3E and the corresponding chiral Compound 4E. Then, the chiral Compound 4E obtained through hydrolysis can be compared with a standard chiral sample of Compound 4E to determine the chiral purity of the chiral Compound 5E. The determination can be conducted by using a chiral HPLC.

### Example 33: Preparation of Tris[(2S,3S)-2-amino-3-methyl-N-(2-morpholinoethyl)pentanamide]tetra(monohydrogensulfate), monosulfate (LM11A-31)

To a solution of LM11A-31 free base (25 g, 0.103 mol) dissolved in absolute, anhydrous ethanol (250 mL, 10 vol) cooled in an ice-water bath was slowly added concentrated sulfuric acid (4 mL, 75 mL) by dropwise addition. Precipitation immediately occurred causing stirring to stop. The ice-water batch was removed and the addition of ethanol (200 mL) and isopropanol (225 mL) was necessary to restart stirring. The remaining required sulfuric acid (7 mL, 131 mmol) was slowly added in an ethanol:isopropanol solution (2:1, 75 mL). An exotherm (20.8° C.-24.0° C.) was observed. The white slurry was allowed to stir overnight over positive pressure. The mixture was then filtered washing with isopropanol (150 mL) and dried under high vacuum (35° C.-40° C.) to afford 33.6 g (75% yield) of Tris[(2S,3 S)-2-amino-3-methyl-N-(2-morpholinoethyl)pentanamide]tetra(monohydrogensulfate), monosulfate as a white solid.

### Materials and Methods for Examples 34-39

### Materials

Rabbit anti-glyceraldehyde 3-phosphate dehydrogenase (GAPDH; 1:2000), goat anti-actin (1:2000), rabbit anti-phospho-ERK (pY204; 1:2000) and mouse anti-ERK (1:5000) antibodies were purchased from Santa Cruz Biotechnologies. The donkey horseradish peroxidase (HRP)-conjugated secondary antibodies (1:1000) were purchased from Jackson ImmunoResearch. Rabbit anti-p75NTR (1:500), anti-TrkB (1:1000), and mouse anti-NeuN (1:500) antibodies were purchased from EMD Millipore. Rabbit anti-phospho-TrkB (pY490; 1:200) antibodies were purchased from Cell Signaling Technology. Mouse anti-glial fibrillary acidic protein (GFAP; 1:1000) antibodies, phosphatase inhibitor cocktails 1 and 2, and primers for the PCR were purchased from Sigma-Aldrich. Enhanced chemiluminescence (ECL) was purchased from GE Healthcare. The secondary antibodies Alexa Fluor 488-labeled donkey anti-rabbit and Alexa Fluor 594-labeled donkey anti-mouse and NuPAGE Bis-Tris pre-casted gels were purchased from Invitrogen. Rabbit (1:5000) anti-green fluorescence protein (GFP) antibodies were purchased from Abcam. The bicinchoninic acid (BCA) protein assay kit was obtained from Pierce. Human BDNF was purchased from Sigma-Aldrich. LM11A-31 was custom synthesized by Ricerca Biosciences.

Animals

Male Long-Evans rats (250 g at the time of purchase) were obtained from Harlan. Animals were housed individually under a light/dark cycle of 12 h, with lights on at 7:00 A.M. and food and water available ad libitum. All animal procedures were approved by the University of California, San Francisco (UCSF), and Gallo Center and the UCSF Institutional Animal Care and Use Committee, and were conducted in agreement with the Guide for the Care and Use of Laboratory Animals (National Research Council) Gallo Center and Association for Assessment and Accreditation of Laboratory Animal Care (UCSF) guidelines.

### Preparation of Solutions

Alcohol solution for the drinking experiments was prepared from absolute anhydrous alcohol (190 proof) diluted to 10% or 20% alcohol (v/v) in tap water. For systemic administration, alcohol was diluted to 20% alcohol (v/v) in saline (0.9% NaCl; Hospira). Sucrose solution was diluted to 1% sucrose (v/v) in tap water.

Production of Lentivirus Expressing shRNA Targeting p75NTR

The 21 nucleotide shRNA sequence targeting p75NTR (shp75NTR) 5′-GGACCTATCTGAGCTGAAAGC-3′ was selected. A scrambled sequence (shSCR), 5′-GAA GCA ACT CGT CTG GAC AGT-3′ was designed using a public siDesign website and was used as a nontargeting shRNA control. Both sequences were incorporated into a stem-loop structure. Synthesized oligonucleotides were annealed and subcloned into the HpaI and XhoI restriction sites in the recombinant lentiviral vector pLL3.7, which also expresses enhanced GFP (EGFP). The expression of shRNA was driven by the U6 promoter and the expression of EGFP by the CMV promoter. The pLL3.7-shp75NTR or pLL3.7-shSCR plasmid was transfected into HEK293T (Clonetech) cells along with the packaging plasmids, psPAX2 and pMD2.G, using Lipofectamine 2000. Forty-eight hours after transfection, the supernatant was collected, and lentiviral particles were purified by ultracentrifugation (26000 rpm, 90 minutes at 4° C.). Titers were determined using the HIV-1 p24 antigen ELISA kit (ZeptoMetrix) per the manufacturer's instructions. The titer of viruses used in the studies was 2×10⁷ pg/ml.

### Preparation of Synaptosomal Fraction

The DLS and the dorsomedial striatum (DMS) were dissected, and crude synaptosomal fractions were prepared as described by Gibb et al. [J Neurocem 119:879-889 (2011)]. Immediately after being collected, tissue was homogenized in a glass homogenizer containing 500 μl of ice-cold Krebs-sucrose buffer [containing (in mM) 125 NaCl, 1.2 KCl, 1.2 MgSO₄ 1.2 CaCl₂, 22 Na₂CO₃, 1.2 NaH₂PO₄, 10 glucose, and 320 sucrose, pH 7.4] in the presence of protease and phosphatase inhibitors. The homogenate was centrifuged at 1000×g at 4° C. for 10 minutes to pellet heavy membranes and debris (P1). The supernatant (S1) was collected and was centrifuged at 16,000×g at 4° C. for 20 minutes to pellet the crude synaptosomal fraction (P2). P2 was resuspended in 100 μl RIPA buffer. Total protein concentration was determined using BCA protein assay kit.

### Western Blot Analysis

Equal amounts of homogenates from individual rats (25 μg) were resolved on NuPAGE 4-12% Bis-Tris gels and transferred onto nitrocellulose membranes. Blots were incubated for 1 hour at room temperature in blocking solution [5% (w/v) nonfat milk in Tris-buffered saline containing 0.1% (v/v) Tween 20] and then incubated overnight at 4° C. in a blocking solution including the primary antibodies. Membranes were then washed and probed with HRP-conjugated secondary antibodies for 2 h at room temperature. Bands were visualized using ECL and quantified by ImageJ (NIH).

### Immunochemistry

Four weeks after virus infusion, rats were deeply anesthetized with Euthasol (Virbac) and perfused with 0.9% NaCl, followed by 4% paraformaldehyde (PFA) in phosphate buffer, pH 7.4. Brains were removed and fixed in the same fixative for 2 hours, and then transferred to PBS at 4° C. The following day, brains were transferred into 30% sucrose and stored at 4° C. until the brain sank to the bottom of the tube. Frozen 50-μm-thick coronal sections were cut on a cryostat (Microm; Thermo Fisher Scientific) and collected into 24-well dishes. Free-floating sections containing the injection site in the DLS were selected. Coronal sections were blocked with 5% normal donkey serum in PBS for 1 h and then incubated for 24 h at 4° C. on an orbital shaker with antibodies for either a neuronal marker (anti-NeuN antibody) or a glial marker (anti-GFAP antibody) in combination with the anti-GFP antibody, diluted in PBS plus 3% bovine serum albumin and 0.05% Triton X-100. The sections were then washed three times for 5 min each in PBS followed by incubation for 4 h with the secondary antibodies Alexa Fluor 488-labeled donkey anti-rabbit and Alexa Fluor 594-labeled donkey anti-mouse (both at 1:500). After staining, the sections were washed three times for 5 minutes each in PBS and mounted in Vectashield mounting medium. For each subject, the infected area was verified in 50 m coronal sections using Zeiss LSM 510 META laser confocal microscope.

### Systemic Administration of Alcohol

Rats were habituated to the intraperitoneal administration procedure with a daily injection of saline (0.9% NaCl) for 3 days. Rats then received an intraperitoneal administration of alcohol (1.5 g/kg) or saline 30 minutes before brain tissue collection.

### Excessive Alcohol Intake: Intermittent Access to 20% Alcohol

At 8 weeks of age, animals were given 24 hours of access to one bottle of 20% alcohol (v/v) and one bottle of tap water concurrently for a total of 21 sessions of 24 hours during a period of 7 weeks. Drinking sessions started at 12:00 P.M. on Monday, Wednesday, and Friday, with 24 or 48 hours (weekend) of alcohol deprivation between the drinking sessions. The water and alcohol bottles were weighed before the beginning of each drinking session, immediately after the first 30 minutes of access to alcohol and at the end of the session. Rats with escalation of alcohol consumption from session 1 to session 18, reaching a baseline of drinking that was higher than 3.5 g/kg/24 h, were selected for the studies. Between 65% to 70% of the rats showed escalation of drinking. The rats that did not meet the latter criteria were excluded from the study.

### Moderate Alcohol Intake: Continuous Access to 10% Alcohol

The rats underwent continuous access to one bottle of 10% alcohol (v/v) and one bottle of water in their home cage for 21 consecutive days (3 weeks). Alcohol and water consumption were recorded daily. Continuous access to 10% alcohol does not lead to escalation of alcohol intake; i.e., there is no difference in the amount of consumed alcohol between day 1 and day 18 (N.M. and D.R., unpublished observation) and all rats were included in the study.

### Operant Self-Administration of Alcohol

After 5 weeks of the intermittent access (IA) to 20% alcohol 2BC paradigm (IA20%-2BC), the rats were trained to self-administer alcohol. The self-administration chambers (Med-Associates) contain two levers: an active lever (the alcohol lever), for which presses result in the delivery of 0.1 ml of the alcohol solution (v/v), and an inactive lever, for which presses are counted but no alcohol solution is delivered. Rats were trained under a fixed ratio 3 schedule; i.e., rats had to press three times to receive one reward. Sessions lasted for the duration of 1 h at the beginning of the training and 30 min after the first 2 weeks. The number of presses on the levers and the number of alcohol deliveries were recorded using MED-PC IV software (Med-Associates).

### Intermittent Access to Sucrose

The procedure was similar to the IA20%-2BC paradigm described above, except that a sucrose (1%) solution was substituted for alcohol. Rats were tested for sucrose intake in response to intra-DLS administration of LM11A-31 or vehicle 1 week after the conclusion of the alcohol-drinking study. Animals were trained to drink sucrose for 2 weeks before the test day. An independent cohort of rats underwent IA-2BC sucrose-drinking paradigm for 7 weeks before systemic administration of LM11A-31.

### Surgical Procedures

Rats with a stable baseline level of drinking in the alcohol self-administration or IA-20%-2BC paradigms underwent surgeries under isoflurane (Baxter) anesthesia. Rats were weighed and monitored daily after the surgery to ensure the health and well-being of each animal.

Infusion of virus. Two holes were drilled above the sites of injection to allow the introduction of injector cannula (acute internal cannula, C315IA; diameter, 33G; PlasticsOne). The injectors were connected to 25 μl Hamilton syringes (#1702), and infusions were controlled by an automatic pump (Harvard Apparatus). Rats received three injections of 1 μl of solution containing the virus per side at a rate of 0.2 μl/min. Injectors remained in place for 10 additional minutes. The coordinates used for the injections into the DLS were all relative to Bregma and were as follows: injection 1, +1.2 mm anterior, 3.5 mm lateral to the medial suture, −4.5 mm ventral to the skull surface; injection 2, +0.2 mm anterior, 3.5 mm lateral to the medial suture, −5.5 mm ventral to the skull surface; injection 3, +0.2 mm anterior, 3.5 mm lateral to the medial suture, −4.5 mm ventral to the skull surface. After 1 week of recovery, alcohol-drinking procedure was resumed.

Cannula implantation and microinfusions of BDNF and LM11A-31. Four holes were drilled for screws, and two holes were drilled for cannulae implantation (single cannula, C315GA; PlasticsOne). The coordinates for the DLS were +1.2 mm anterior to Bregma and 3.5 mm lateral to the medial suture. The cannulae were implanted into the lateral part of the dorsal striatum (−4.2 mm from the skull surface) and fixed with dental cement. After 1 week of recovery, the alcohol-drinking procedure resumed, and subjects were habituated to the microinjection procedure with two sham injections (injectors were lowered but no infusion took place). The experimental microinfusions began after each rat reacquired a stable level of drinking. BDNF, LM11A-31, or PBS was infused via a 25 μl Hamilton syringe (#1702), 3 h (BDNF) or 2 h (LM11A-31) before the beginning of the alcohol-drinking session. BDNF (0.75 μg/1 μl) or LM11A-31 (30 μg/1 μl) was infused over 2 minutes in each side of the brain. The injectors extended 0.5 mm below the tip of the cannula and remained in position for an additional 2 minutes to allow for full diffusion from the injector tip.

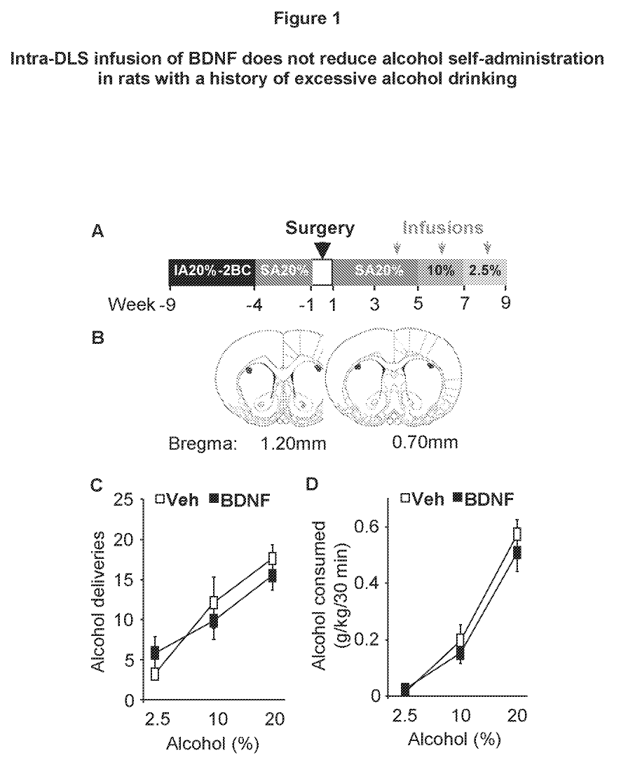

In the experiment testing alcohol self-administration after intra-DLS administration of BDNF (FIGS. 1A-D), each concentration of alcohol was self-administered for a total period of 2 weeks, and microinfusions were performed 4 days after the change in alcohol concentration. All subjects received each treatment in a counterbalanced manner within each alcohol concentration.

In the experiments testing intra-DLS administration of LM11A-31, all subjects received each treatment in a counterbalanced manner, with one microinfusion per week. Two alcohol-drinking sessions without treatment were carried out between drug administrations to allow intake to return to baseline.

### Systemic Administration of LM11A-31

Before the beginning of experiments, rats were habituated for 3 days to intraperitoneal administration by daily injections of saline (0.9% NaCl). LM11A-31 (50-150 mg/kg) or vehicle (0.9% saline) was administered intraperitoneally 2 hours before the beginning of the drinking session. Test days occurred once a week, with two alcohol-drinking sessions without treatment that were performed between injections to allow intake to return to baseline. For the highest dose of LM11A-31, vehicle and drug were tested in different animals.

### Histology

Rats implanted with cannulae were perfused transcardially with 4% PFA. Fixed brains were frozen, and 50 μm coronal slices were cut and examined for cannulae placement using a bright-field microscope.

### Data Analysis

Data are expressed as mean±SEM. Biochemical data were analyzed with a two-tailed unpaired t test. Behavioral data were analyzed with a two-tailed paired or unpaired t test or by one- or two-way ANOVA with or without repeated measures (RM), depending on the experiment, followed by the Student-Newman-Keuls (SNK) test when indicated by significant effects of treatments or interactions. Statistical significance was set at p<0.05.

### Example 34: BDNF in the DLS does not Alter Alcohol Consumption in Rats with a History of Excessive Alcohol Drinking and Withdrawal

It was shown previously that the administration of exogenous BDNF into the DLS reduces operant alcohol self-administration in rats with history of moderate (10%) alcohol drinking, whereas knockdown of BDNF in the DLS increases 10% alcohol self-administration and intake. These data suggest that the BDNF signaling pathway in the DLS gates the level of alcohol intake.

To test the possibility that BDNF signaling in the DLS is disrupted after chronic excessive alcohol drinking, the ability of the neurotrophic factor to control alcohol self-administration in rats with a history of excessive alcohol drinking was tested. The rats underwent 5 weeks of an intermittent access to 20% alcohol in a 2BC drinking paradigm (IA20%-2BC paradigm) and were trained to lever press for a 20% alcohol solution during 30 min sessions (FIG. 1A). The intermittent access to 20% alcohol in a 2BC drinking paradigm was used in order to induce escalation to excessive alcohol consumption, and after 5 weeks of binge and withdrawal, the rats were trained to self-administer alcohol in an operant self-administration paradigm. After achieving a stable baseline, BDNF (0.75 μg/side) or vehicle (Veh; PBS) was infused into the dorsolateral striatum (DLS) (FIG. 1B) 3 hours before the beginning of the operant self-administration sessions. The self-administration of three alcohol concentrations was measured. The concentration of alcohol was decreased (from 20% to 10% and from 10% to 2.5%) every 2 weeks.

It was found that intra-DLS administration of BDNF did not decrease the number of alcohol deliveries (FIG. 1C; two-way RM-ANOVA, treatment effect, F_{(1,12)}=0.11, p=0.74) or the amount of alcohol consumed (FIG. 1D; two-way RM-ANOVA, treatment effect, F_{(1,12)}=0.53, p=0.48), independently of the concentrations of alcohol (2.5, 10, and 20%). These findings suggest that repeated access to high amounts of alcohol together with repeated episodes of withdrawal preclude the ability of BDNF in the DLS to gate the level of alcohol self-administration.

### Example 35: p75NTR Localization in the DLS is Altered in Rats with a History of Excessive Alcohol Drinking and Withdrawal

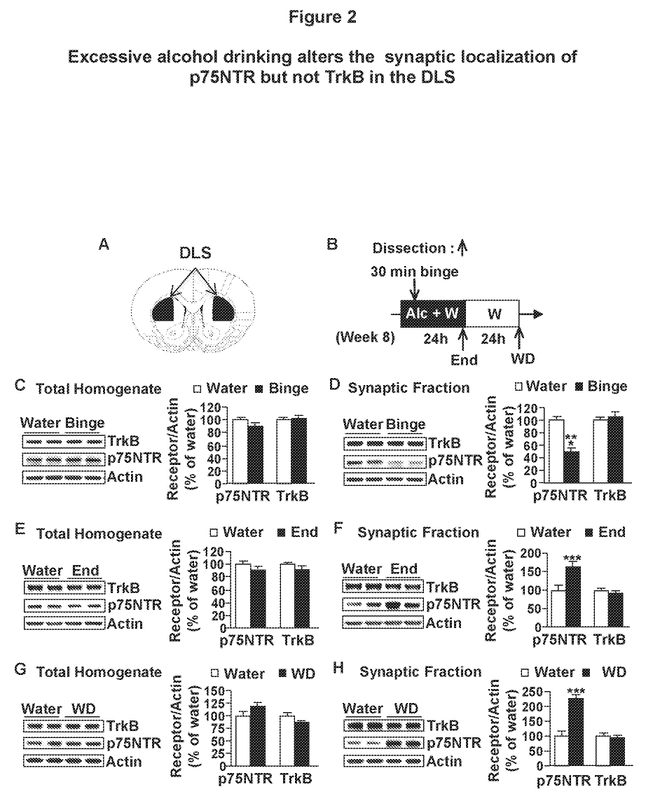

The effect of long term exposure to alcohol on BDNF/TrkB signaling in the DLS was studied. The levels of TrkB in the DLS of rats that were subjected to repeated cycles of binge drinking and withdrawal were examined. After 7 weeks of IA20%-2BC, the DLS (FIG. 2A) was dissected after the first 30 min of alcohol access (binge), immediately at the end of the alcohol-drinking session (end) or after 24 h of alcohol deprivation (withdrawal; FIG. 2B). Individual alcohol consumption values before dissection are reported in Table II.

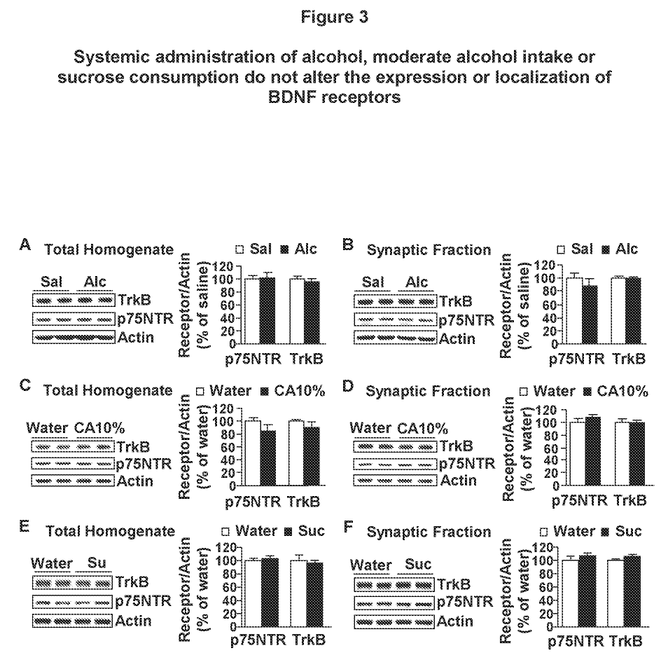

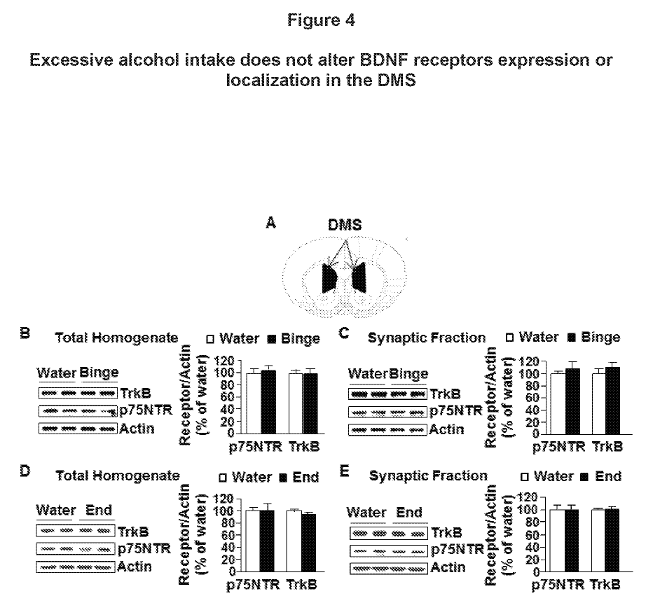

TABLE II
Individual alcohol intake date for the biochemical experiments

| | | Last drinking | |
|---|---|---|---|
| | Rat number | session (g/kg/24 h) | Binge (g/kg/30 min) |
| FIGS. 2C,D, 4B,C; | 1 (FIGS. 2, 4) | 11.17 | 0.3 |
| drinking procedure, | 2 (FIGS. 2, 4) | 7.25 | 0.07 |
| IA20%-2BC; harvest | 3 (FIGS. 2, 4) | 4.79 | 0.43 |
| time point, 30 min | 4 (FIGS. 2, 4) | 5.19 | 0.31 |
| binge | 5 (FIGS. 2, 4) | 9.7 | 3.06 |
| | 6 (FIGS. 2, 4) | 6.35 | 2.54 |
| | 7 (FIGS. 2, 4) | 5.35 | 1.24 |
| | 8 (FIGS. 2, 4) | 4.05 | 0.3 |
| | 9 (FIG. 2) | 6.05 | 1.22 |
| | 10 (FIG. 2) | 9.69 | 1.59 |
| | Mean ± SEM | 6.96 ± 0.77 | 1.11 ± 0.33 |
| FIGS. 2E,F, 4D,E; | 1 | 9.05 | |
| drinking procedure, | 2 | 5.24 | |
| IA20%-2BC; harvest | 3 | 4.91 | |
| time point, end of | 4 | 4.29 | |
| drinking | 5 | 7.4 | |
| | 6 | 3.81 | |
| | 7 | 5.87 | |
| | 8 | 4 | |
| | Mean ± SEM | 5.57 ± 0.64 | |
| FIG. 2G,H; drinking | 1 | 5.28 | |
| procedure, IA20%- | 2 | 7.18 | |
| 2BC; harvest time | 3 | 3.53 | |
| point, 24 h withdrawal | 4 | 3.75 | |
| | 5 | 9.06 | |
| | 6 | 6.5 | |
| | 7 | 5.35 | |
| | 8 | 4.81 | |
| | Mean ± SEM | 5.68 ± 0.65 | |
| FIG. 2C,D; drinking | 1 | 0.98 | |
| procedure, CA10%- | 2 | 1.85 | |
| 2BC; harvest time | 3 | 1.28 | |
| point, end of drinking | 4 | 1.07 | |
| | 5 | 1.06 | |
| | 6 | 0.28 | |
| | 7 | 0.68 | |
| | 8 | 0.22 | |
| | Mean ± SEM | 0.93 ± 0.19 | |
| FIG. 3E,F; drinking | 1 | 45.99 | 1.45 ml/kg/30 min |
| procedure, IA1% | 2 | 47.77 | 4.58 ml/kg/30 min |
| sucrose-2BC; harvest | 3 | 54.48 | 6.72 ml/kg/30 min |
| time point, 30 min | 4 | 44.71 | 8.75 ml/kg/30 min |
| binge | Mean ± SEM | 48.24 ± 2.17 | 5.38 ± 1.56 ml/kg/30 min |

No differences were observed in total homogenate levels for TrkB after alcohol binge drinking (FIG. 2C; t₍₂₂₎=−0.57, p=0.58), at the end of the last 24 h of alcohol-drinking session (FIG. 2E; t₍₁₄₎=1.14, p=0.28), or after 24 h of withdrawal (FIG. 2G; t₍₁₄₎=1.57, p=0.14). The levels of TrkB in the synaptosomal fraction were also unaltered in the DLS of alcohol-drinking animals following binge (FIG. 2D; t₍₂₂₎=−1.50, p=0.15), 24 h of consumption (FIG. 2F; t₍₁₄₎=1.96, p=0.07), and after 24 h of withdrawal (FIG. 21H; t₍₁₄₎=0.56, p=0.58) compared to control animals that consumed water only. The levels of TrkB phosphorylation and the phosphorylation of ERK1/2 in total homogenate were tested. It was found that neither TrkB nor ERK1/2 were phosphorylated, and thus activated, in response to binge alcohol drinking (p-TrkB, t₍₇₎=1.23, p=0.26, n=4-5/group; p-ERK1/2, t₍₁₅₎=1.20, p=0.24, n=8-9) or after 24 h of withdrawal (p-TrkB, t₍₆₎=0.42, p=0.21, n=4/group; p-ERK1/2, t₍₁₂₎=1.65, p=0.13, n=7/group). These data suggest that long-term excessive drinking and withdrawal do not alter the expression or activity of TrkB.

It was examined whether a history of excessive alcohol intake alters the levels and/or localization of the low-affinity neurotrophin receptor p75NTR. The protein levels of p75NTR in total homogenate of DLS were unaltered in response to a binge drinking session (FIG. 2C; t₍₂₁₎=1.99, p=0.06), at the end of the last 24 h of alcohol-drinking session (FIG. 2E; t₍₁₄₎=1.81, p=0.09), or after 24 h of withdrawal (FIG. 2G; t₍₁₄₎=−1.87, p=0.08). In contrast, a 30 min binge alcohol-drinking session decreased the synaptosomal localization of p75NTR in the DLS (FIG. 2D; t₍₂₁₎=12.18, p<0.0001), whereas the membranal level of p75NTR was significantly increased at the end of the final 24 h drinking session compared to water-consuming rats (FIG. 2F; t₍₁₄₎=−5.18, p<0.0001), which was maintained even after 24 h of withdrawal (FIG. 2H; t₍₁₄₎=−10.10, p<0.0001). The results suggest that the synaptosomal localization of p75NTR is dynamic, changing over the course of a 24 h drinking session and withdrawal.

### Example 36: Expression Levels and Localization of p75NTR are Unaltered in the DLS after Acute Alcohol Administration and in Response to Moderate Alcohol Consumption or Sucrose Intake

It was examined whether the alterations in p75NTR localization are a consequence of the history of high levels of voluntary alcohol intake or are due to the acute pharmacological actions of alcohol per se. p75NTR levels in the rat DLS after an acute systemic administration of a nonhypnotic dose of alcohol (1.5 g/kg) were tested. Acute alcohol exposure did not alter the total protein levels of p75NTR (FIG. 3A; t₍₁₄₎=−0.42, p=0.68), and the synaptosomal levels of the receptor were also unaltered (FIG. 3B; t₍₁₄₎=0.99, p=0.34). TrkB levels in both the total homogenate (FIG. 3A; t₍₁₄₎=1.08, p=0.30) and the synaptosomal fraction (FIG. 3B; t₍₁₄₎=−0.29, p=0.78) were also unaffected in response to the systemic administration of alcohol.

It was tested whether the alterations in the synaptosomal p75NTR levels are specific to excessive alcohol drinking and are not a result of prolonged alcohol exposure per se was examined by measuring the levels of both receptors in the DLS of rats that underwent a paradigm of moderate alcohol intake. Rats had continuous access to 10% alcohol for 21 d (i.e., for the same number of alcohol access sessions as the rats that were drinking 20% alcohol), and the DLS was dissected at the end of the last 24 h drinking session. The individual alcohol consumption values before dissection are reported in Table II. The levels of both receptors in the total homogenate (FIG. 3C; TrkB, t₍₁₄₎=1.39, p=0.19; p75NTR, t₍₁₄₎=1.62, p=0.13) and in the synaptosomal fraction (FIG. 3D; TrkB, t₍₁₄₎=0.20, p=0.84; p75NTR, t₍₁₄₎=−1.35, p=0.20) were unaltered in the DLS of rats consuming moderate levels of alcohol compared to control water-only-consuming rats.

It was tested whether the consumption of another rewarding substance (e.g., sucrose) attenuates the expression and/or localization of the BDNF receptors was determined. Rats underwent 7 weeks of IA1% sucrose 2BC, and the DLS was dissected immediately after the last 30 min of drinking. Individual sucrose consumption values before dissection are reported in Table II. No changes were observed in the total (FIG. 3E; TrkB, t₍₆₎=0.54, p=0.61; p75NTR, t₍₆₎=−0.47, p=0.65) or synaptosomal (FIG. 3F; TrkB, t₍₆₎=−1.45, p=0.20; p75NTR, t₍₆₎=−1.37, p=0.22) homogenates of the DLS of sucrose-drinking rats compared to water-only-consuming animals. The results demonstrate that the changes in p75NTR localization are specific to a regime of alcohol consumption, i.e., a history of high, binge-like levels of intake with intermittent access and withdrawal periods.

### Example 37: TrkB and p75NTR Expression and Localization are Unaltered in the DMS in Response to Alcohol Consumption

To determine whether altered p75NTR localization was specific to the DLS, the expression and/or localization of TrkB and p75NTR in response to alcohol in the DMS (FIG. 4A), a striatal region where endogenous BDNF does not control alcohol self-administration (Jeanblanc et al., 2009), was examined. Individual alcohol consumption values before dissection are reported in Table II. The total levels of TrkB (FIG. 4B; t₍₁₄₎=−0.07, p=0.94) and p75NTR (FIG. 4B; t₍₁₄₎=−0.70, p=0.49) and the synaptosomal localization of both receptors (FIG. 4C; TrkB, t₍₁₄₎=−2.092, p=0.06; p75NTR; t₍₁₄₎=−0.46, p=0.65) were unchanged in the DMS of rats after a 30 min binge alcohol intake session. Similarly, no change was observed after a 24 h alcohol drinking session in the total homogenate (FIG. 4D; TrkB, t₍₁₄₎=2.2, p=0.06; p75NTR, t₍₁₄₎=−0.05, p=0.96) and synaptosomal fraction (FIG. 4E; TrkB, t₍₁₄₎=−0.91, p=0.38; p75NTR, t₍₁₄₎=−0.06, p=0.95). The results suggest that the changes in p75NTR localization in response to binge drinking of, and withdrawal from, excessive alcohol intake are specific to the DLS.

### Example 38: Knockdown of p75NTR Expression Attenuates Excessive Alcohol Drinking

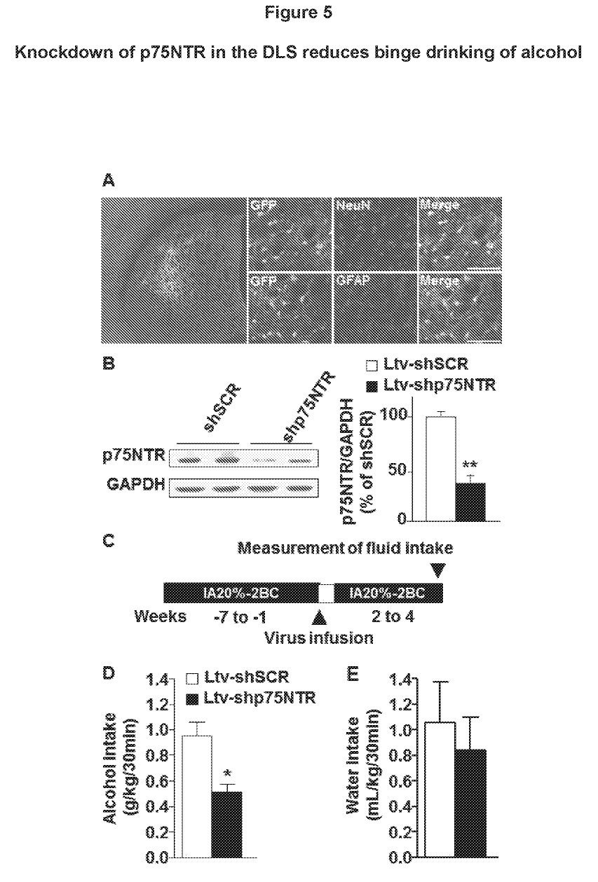

To test the possibility whether the breakdown in BDNF signaling upon excessive alcohol consumption may be due to the increased functional contribution of p75NTR, a lentivirus expressing EGFP and a shRNA sequence targeting p75NTR (Ltv-shp75NTR), as well as a virus expressing a scrambled p75NTR shRNA sequence (Ltv-shSCR) were generated. Ltv-shp75NTR or Ltv-shSCR was bilaterially infused into the DLS of the rats at a titer of 2×10⁷ pg/ml. Infusion of Ltv-shp75NTR into the DLS of rats led to a high level of virus infection in DLS neurons but not in glia, as shown by colocalization of GFP and NeuN but not GFAP staining (FIG. 5A). The protein levels of p75NTR were significantly reduced in the DLS of rats infected with the Ltv-shp75NTR compared with rats infected with Ltv-shSCR control, as measured 4 weeks after virus infusion (FIG. 5B; t₍₆₎=5.40, p=0.001). The consequences of p75NTR knockdown on alcohol intake were tested. After 7 weeks of the IA20%-2BC paradigm, rats received a bilateral infusion of either Ltv-shSCR or Ltv-shp75NTR into the DLS, and alcohol intake was determined 4 weeks after virus infection (FIG. 5C). As shown in FIGS. 5, D and E, knockdown of p75NTR in the DLS significantly reduced binge-like alcohol drinking (FIG. 5D; t₍₁₂₎=3.62, p=0.01), without affecting concurrent water intake (FIG. 5E; t₍₁₂₎=0.52, p=0.62). Thus, p75NTR in the DLS contributes to the maintenance of excessive alcohol intake.

### Example 39: The p75NTR Modulator, LM11A-31, Reduces Excessive Alcohol Consumption

The small nonpeptide p75NTR ligand LM11A-31 modulates p75NTR signaling (Massa et al., 2006; Longo and Massa, 2013; Tep et al., 2013) by disrupting the binding between p75NTR and neurotrophins, including BDNF, and by modulating its intrinsic signaling actions (Massa et al., 2006; Longo and Massa, 2013). LM11A-31 blocks p75NTR-mediated neuronal death (Knowles et al., 2013; Tep et al., 2013) and decreases the phosphorylation of Jun kinase, a downstream effector of p75NTR (Shi et al., 2013).

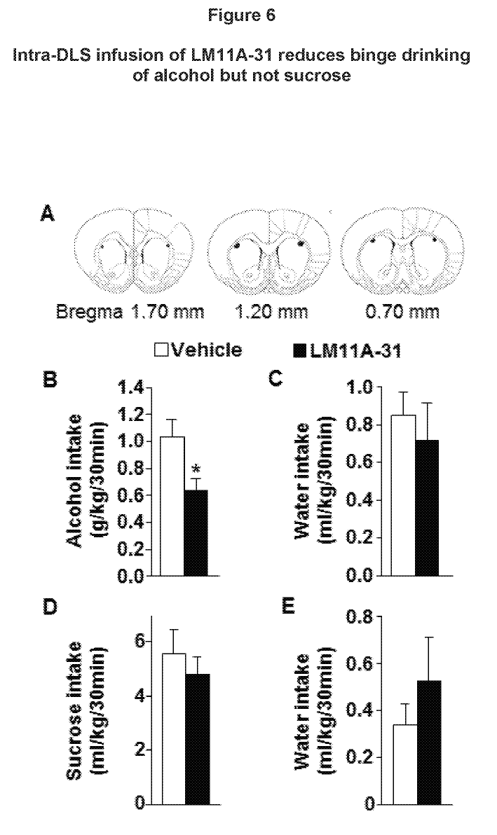

To test whether LM11A-31 reduces excessive alcohol drinking, LM11A-31 was infused into DLS (FIG. 6A) 2 h before the beginning of a 30 min alcohol binge-drinking session. As shown in FIGS. 6, B and C, intra-DLS administration of LM11A-31 significantly reduced binge-like alcohol intake (FIG. 6B; t(10)=2.35, p<0.05) without changing concurrent water intake (FIG. 6C; t₍₁₀₎=0.76, p=0.47). Importantly, the effect of LM11A-31 on alcohol intake was specific, as sucrose intake was unaltered upon infusion of the drug into the DLS (FIG. 6D; sucrose, t₍₉₎=0.85, p=0.41; E, water, t₍₉₎=−0.73, p=0.45). These results indicate that the reduction of binge alcohol drinking by LM11A-31 was unlikely to be due to nonspecific motor or reward sensitivity changes.

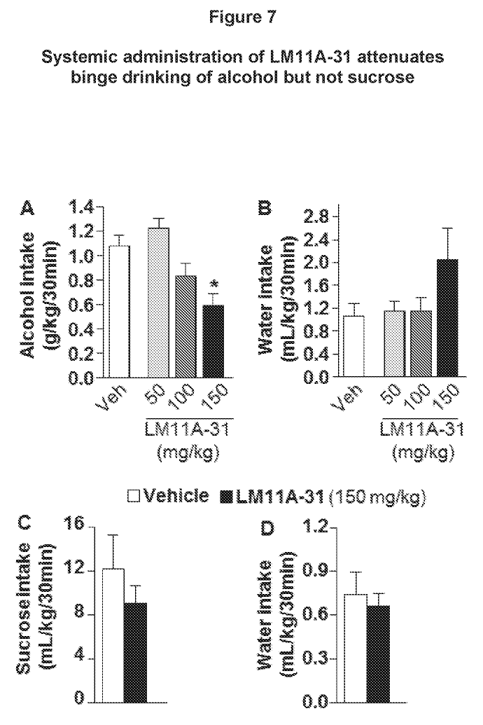

The possibility that LM11A-31 could potentially be developed for the treatment of alcohol abuse disorders was examined by testing the effects of systemic administration of LM11A-31 on alcohol intake. As shown in FIG. 7, modulation of p75NTR-mediated signaling significantly attenuated binge drinking of alcohol (FIG. 7A; one-way ANOVA, F_{(3,56)}=7.12, p<0.001) at the dose of 150 mg/kg (p<0.01), without altering water intake (FIG. 7B; one-way ANOVA, F_{(3,56)}=2.07, p=0.11).

To test whether systemic administration of LM11A-31 attenuates binge drinking of sucrose, rats had IA to 1% sucrose in a 2BC paradigm for 7 weeks, and sucrose consumption was measured after 30 min access following LM11A-31 injection. As shown in FIG. 7C, LM11A-31 (150 mg/kg) did not alter binge consumption of sucrose, and the corresponding water intake was also unaltered (FIG. 7D; sucrose, t₍₁₃₎=0.93, p=0.37; water, t₍₁₃₎=0.39, p=0.71). These data suggest that pharmacological targeting of p75NTR is a potential treatment of alcohol abuse disorder by attenuating alcohol intake without affecting the consumption of a natural rewarding substance.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating an alcohol use disorder, comprising administering to a subject in need thereof an effective amount of a compound represented by Formula III: 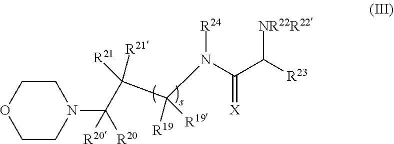
or a salt thereof, wherein:
X is CH₂, NH, O or S;
s is 0, 1, 2, 3 or 4;
each of R¹⁹, R^{19′}, R²⁰, R^{20′}, R²¹, R^{21′}, R²², R^{22′} and R²⁴ is independently selected at each occurrence from hydrogen and optionally substituted alkyl; or
R²⁰ and R^{20′} taken together form ═O, ═S, or ═CH₂; or
R²⁰ and R²¹ taken together with the atoms to which they are attached form an optionally substituted cycloalkyl; or
R²⁰ and R²¹ taken together with the atoms to which they are attached form an optionally substituted aryl; or
R¹⁹ and R²⁰ taken together with the atoms to which they are attached form an optionally substituted cycloalkyl; or
R¹⁹ and R²⁰ taken together with the atoms to which they are attached form an optionally substituted aryl; and
R²³ is hydrogen, optionally substituted alkyl, optionally substituted cycloalkyl or optionally substituted aryl.

2. The method of claim 1, wherein s is 0, 1 or 2.

3. The method of claim 2, wherein s is 0.

4. The method of any one of claims 1 to 3, wherein X is NH, O or S.

5. The method of claim 4, wherein X is O.

6. The method of any one of claims 1 to 5, wherein R²⁰ and R^{20′} are independently selected from hydrogen and optionally substituted C₁-C₆ alkyl.

7. The method of claim 6, wherein R²⁰ and R^{20′} are each hydrogen.

8. The method of any one of claims 1 to 7, wherein R²¹ and R^{21′} are independently selected from hydrogen and optionally substituted C₁-C₆ alkyl.

9. The method of claim 8, wherein R²¹ and R^{21′} are each hydrogen.

10. The method of any one of claims 1 to 9, wherein R²² and R^{22′} are independently selected from hydrogen and optionally substituted C₁-C₆ alkyl.

11. The method of claim 10, R²² and R^{22′} are each hydrogen.

12. The method of any one of claims 1 to 11, wherein R²³ is selected from hydrogen, optionally substituted C₁-C₆ alkyl, optionally substituted cycloalkyl and optionally substituted aryl.

13. The method of claim 12, wherein R²³ is selected from optionally substituted C₁-C₆ alkyl.

14. The method of claim 13, wherein R²³ is represented by the structure:

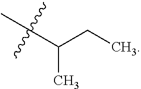 [SMILES: *C(C)CC]

15. The method of any one of claims 1 to 14, wherein R²⁴ is hydrogen or optionally substituted C₁-C₆ alkyl.

16. The method of claim 15, wherein R²⁴ is hydrogen.

17. The method of any one of claims 1 to 16, wherein the compound of Formula III is represented by the structure: 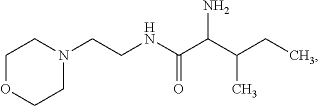 [SMILES: CCC(C)C(N)C(=O)NCCN1CCOCC1]
or a salt thereof.

18. The method of claim 17, wherein the compound of Formula III is represented by the structure: 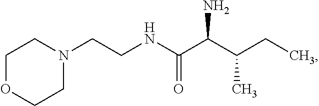 [SMILES: CC[C@H](C)[C@H](N)C(=O)NCCN1CCOCC1]
or a salt thereof.

19. The method of claim 18, wherein the salt of Formula III is represented by Formula

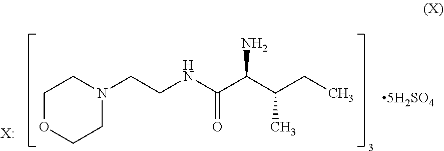

20. The method of any one of claims 1 to 19, wherein said subject has a predisposition to alcoholism.

21. The method of any one of claims 1 to 20, wherein said alcohol use disorder comprises drinking greater than three alcoholic beverages a day.

22. The method of any one of claims 1 to 21, wherein said alcohol use disorder comprises drinking alcoholic beverages three or more days in a week.

23. The method of any one of claims 1 to 22, wherein said subject exhibits one or more symptoms of an alcohol use disorder selected from: hepatic steatosis, alcoholic hepatitis, cirrhosis, gastritis, stomach ulcers, esophageal ulcers, interference with absorption of B vitamins and other nutrients, pancreatitis, high blood pressure, enlarged heart, heart failure, stroke, atrial fibrillation, cardiovascular disease, hypoglycemia, diabetes, erectile dysfunction, interruption of menstruation, nystagmus, weakness of eye muscles, paralysis of eye muscles, thiamine deficiency, dementia, miscarriage, fetal alcohol syndrome, osteoporosis, damaged bone marrow, low platelet count, numbness and pain in body, disordered thinking, short-term memory loss, weakened immune system, infectious disease, cancer, anemia, depression, seizures, gout, nerve damages, and combinations thereof.

24. The method of any one of claims 1 to 23, wherein said subject in need of treatment is a participant in an alcohol use management program.

25. The method of any one of claims 1 to 24, wherein said alcohol use disorder involves increased synaptosomal localization of p75NTR.

26. The method of claim 25, wherein said p75NTR is localized in the DLS.

27. The method of any one of claims 1 to 26, wherein administering said compound or salt modulates p75NTR levels.

28. The method of any one of claims 1 to 27, wherein administering said compound or salt attenuates alcohol intake of said subject as compared with the frequency of alcohol intake prior to administering said compound or salt.

29. The method of any one of claims 1 to 28, wherein administering said compound or salt attenuates alcohol intake of said subject as compared with the amount of alcohol intake before administering said compound or salt thereof.

30. The method of claim 29, wherein administering said compound or salt attenuates alcohol intake of said subject by about 20% or more as compared with the amount of alcohol intake before administering said compound or salt thereof.

31. The method of any one of claims 1 to 30, wherein said compound or salt is administered to said subject at least about once a week.

32. The method according to claim 31, wherein said compound or salt is administered to said subject at least about twice a week.

33. The method of claim 32, wherein said compound or salt is administered daily or every other day to said subject.

34. The method of any one of claims 1 to 33, wherein said compound or salt is administered before, during, and/or after a trigger event.

35. The method of claim 34, wherein a trigger event is selected from attending an event with alcoholic beverages, exposure to a stressful situation, and the end of a work day.

36. The method of any one of claims 1 to 35, wherein administering said compound does not affect consumption of food or non-alcoholic beverages.

37. The method of any one of claims 1 to 36, wherein said alcohol use disorder is selected from alcohol abuse, alcohol dependence, and alcoholism.

38. The method according to claim 37, wherein said alcohol use disorder is an alcohol abuse disorder.

39. The method of any one of claims 1 to 38, wherein said method further comprises administering one or more additional therapeutic agents selected from disulfiram (Antabuse®), oral naltrexone, extended-release naltrexone (Vivitrol®), and acamprosate (Campral®).

40. The method of any one of claims 1 to 39, further comprising administering behavioral therapy to said subject.
